(19) United States
(12) Patent Application Publication
(10) Pub. No.: US 2010/0290996 A1
(43) Pub. Date: Nov. 18, 2010

(54) METHODS AND COMPOSITIONS BASED ON CULTURING MICROORGANISMS IN LOW SEDIMENTAL FLUID SHEAR CONDITIONS

(75) Inventors: Cheryl A. Nickerson, Phoenix, AZ (US); James W. Wilson, Wilmington, DE (US); Mark C. Ott, Houston, TX (US); Eric A. Nauman, West Lafayette, IN (US); Michael J. Schurr, Aurora, CO (US); Mayra A. Nelman-Gonzalez, Seabrook, TX (US); Shameema Sarker, Chandler, AZ (US)

Correspondence Address: GEORGE A LEONE, SR;CITADEL PATENT LAW, 9124 Gravelly Lake Drive SW, SUITE 102, Lakewood, WA, 98499, US

(73) Assignee: Arizona Board of Regents, for and on behalf of Arizona State University, Tempe, AZ (US)

(21) Appl. No.: 12/676,209

(22) PCT Filed: Sep. 10, 2008
(86) PCT No.: PCT/US08/75818

§ 371 Date: Jul. 26, 2010

Related U.S. Application Data
(60) Provisional application No. 60/993,208, filed on Sep. 10, 2007.
(60) Provisional application No. 61/079,160, filed on Jul. 9, 2008.
(60) Provisional application No. 61/032,252, filed on Feb. 28, 2008.
(60) Provisional application No. 61/045,419, filed on Apr. 16, 2008.

(51) Int. Cl. A61K 49/00; A61K 39/112; C12Q 1/68; G01N 33/53; C12Q 1/02; C12Q 1/18; C12N 1/20; C12N 1/18; C12N 1/16; C40B 30/04

(52) U.S. Cl. 424/9.2; 424/235.1; 435/6; 435/7.1; 435/29; 435/32; 435/252.8; 435/253.3; 435/253.4; 435/255.2; 435/255.21; 435/255.4; 506/9

(57) ABSTRACT

This invention is directed to applying a low sedimental fluid shear environment to manipulate microorganisms, and to microorganisms and compositions obtained based on such manipulation. Specifically, the present invention provides methods of modifying a molecular genetic or phenotypic characteristic (e.g., virulence, stress resistance or biofilm formation) of a microorganism by culturing in a low sedimental shear environment. One or more ion concentrations in the culture can be modulated in order to inhibit or amplify the extent of the modification. The present invention also provides microorganisms obtained from a low sedimental shear culture, which exhibit modified and desirable phenotypic characteristics, as well as therapeutic, vaccine and bioindustrial products prepared from such microorganisms. Further, the present invention provides methods for identifying molecules that modulate responses of a microorganism to a low sedimental shear environment and for determining the relevance of such molecules to pathogenenicity of the microorganism.

### STATEMENT REGARDING FEDERALLY SPONSORED RESEARCH OR DEVELOPMENT

This invention was made with Government support under Contract No. NASA grant NCC2-1362 awarded by the United States NASA. The Government has certain rights in this invention.

### FIELD OF THE INVENTION

This invention generally relates to microbial culturing. More particularly, the present invention is directed to applying a low sedimental fluid shear environment to manipulate microorganisms. Microorganisms obtained from a low sedimental fluid shear culture, which exhibit modified phenotypic and molecular genetic characteristics, are useful for the development of novel and improved diagnostics, therapeutics, vaccines and bioindustrial products. Further, application of low sedimental fluid conditions to microorganisms permits identification of molecules uniquely expressed under these conditions, providing a basis for the design of new therapeutic targets.

### BACKGROUND OF THE INVENTION

Environmental conditions and crewmember immune dysfunction associated with spaceflight may increase the risk of infectious disease during a long-duration mission. Previous studies using the enteric bacterial pathogen, Salmonella enterica serovar Typhimurium, showed that growth in a ground-based spaceflight analog bioreactor, termed the rotating wall vessel (RWV), induced molecular genetic and phenotypic changes in this organism. Specifically, S. typhimurium grown in this spaceflight analog culture environment, described as low shear modeled microgravity (LSMMG), exhibited increased virulence, increased resistance to environmental stresses (acid, osmotic, and thermal), increased survival in macrophages, and global changes in gene expression at the transcriptional and translational levels. However, our knowledge of microbial changes in response to spaceflight or spaceflight analog conditions and the corresponding changes to infectious disease risk is still limited and unclear. Elucidation of such risks and the mechanisms behind any spaceflight or spaceflight analog-induced changes to microbial pathogens holds the potential to decrease risk for human exploration of space and provide insight into how pathogens cause infections in Earth-based environments.

### SUMMARY OF THE INVENTION

The present invention is directed to applying a low sedimental shear environment to manipulate microorganisms, and to microorganisms and compositions obtained based on such manipulation.

In one aspect, the present invention provides a method for modifying or manipulating a microorganism by culturing the microorganism under low sedimental fluid shear conditions, and harvesting the cultured microorganism. The present method applies to microorganism including but not limited to bacteria, viruses, fungi, protozoa, protists, and worms (such as helminthes), among others. Examples of microorganisms contemplated by the present invention include Salmonella sp. (particularly Salmonella typhimurium), Streptococcus pneumoniae, Pseudomonas aeruginosa, Candida albicans, and Saccharomyces cerevisiae.

In one embodiment, the fluid shear level in the low sedimental shear environment in which the microorganism is being cultured is adjusted to be 100 dynes per cm² or lower, preferably lower than 50 dynes per cm², more preferably lower than 20 dynes per cm², even more preferably 10 dynes per cm² or lower, or even lower than 0.1 dynes per cm².

In another aspect, the present invention provides methods of modifying a phenotypic characteristic of a microorganism by culturing the microorganism in low sedimental shear environments. Phenotypic characteristics that can be modified in accordance with the present invention include but are not limited to, virulence, immunogenicity, stress resistance (such as thermal, acid or oxidative stress resistance), resistance to drugs including anti-microbial compounds (e.g., resistance of a fungus to an anti-fungal compound), ability of a bacterium to form biofilm in culture, metabolic capabilities, among others.

In a specific embodiment, low sedimental shear conditions are applied to an attenuated vaccine strain of a microorganism to enhance the efficacy of the vaccine strain.

In a further aspect, the present invention is directed to the modulation of one or more ion concentrations to manipulate, e.g., to amplify or inhibit, responses of microorganisms to low sedimental shear environments. Ions which can be manipulated to achieve modification of microorganisms include but are not limited to phosphate, chloride, sulfate/sulfur, bromide, nitrate-n, o-phosphate, pH/hydrogen ion, calcium, chromium, copper, iron, lithium, fluoride, magnesium, manganese, molybdenum, nickel, potassium, sodium and zinc, among others.

In another aspect, the present invention provides microorganisms harvested from a low sedimental shear culture.

In still another aspect, the present invention provides a therapeutic composition, including a vaccine composition, comprised of a microorganism obtained from a low sedimental shear culture. Microorganisms suitable for use in the therapeutic composition of the present invention include, for example, Salmonella sp. (particularly Salmonella typhimurium), including an attenuated Salmonella vaccine strain, Streptococcus pneumonia, Pseudomonas aeruginosa, Candida albicans and Saccharomyces cerevisiae, harvested from a culture grown under low sedimental shear conditions.

In another aspect, the present invention provides other compositions formulated with a microorganism obtained from a low sedimental shear culture, useful for various bioindustrial applications.

In a further aspect, the present invention provides a method for identifying a gene of a microorganism which modulates the response of the microorganism to low sedimental shear environments. The method includes culturing the microorganism in a low sedimental shear environment, comparing expression of candidate genes in the microorganism in the low sedimental shear environment relative to control sedimental shear conditions, and identifying genes that exhibit differential expression. Functional categories of genes that have been or can be identified as differentially expressed in accordance with the present invention include, without limitation, virulence genes, iron metabolism genes, ion response or utilization genes, cell surface polysaccharide genes, protein secretion genes, flagellar genes, stress genes, genes coding for ribosomal proteins, genes coding for fimbrial proteins, transcriptional regulator genes, genes involved in extracellular matrix/biofilm synthesis, stress response genes, sigma factors, genes encoding RNA binding proteins, genes encoding small noncoding regulatory RNAs (small RNAs), DNA polymerase genes, RNA polymerase genes, plasmid transfer/conjugation genes, chaperone proteins, carbon utilization genes, Metabolic pathway genes, energy metabolism genes, chemotaxis genes, genes encoding heat shock proteins, genes encoding putative proteins, genes encoding recombination proteins, genes encoding transport system proteins, genes encoding membrane proteins, genes encoding cell wall components (including LPS), housekeeping genes, genes encoding structural proteins and enzymes, and plasmid genes.

In another aspect, the present invention is directed to the use of a host, including during space flight, to study interactions between the host and a microorganism pathogen or an attenuated vaccine strain when both are simultaneously placed in a low sedimental shear environment. The pathogen can, for this purpose, also have been manipulated in an RWV or similar analog. According to this aspect of the invention, hosts include animals, animal analogs, plants, insects, and cell and/or tissue cultures from animals, animal analogs or plants.

### BRIEF DESCRIPTION OF THE DRAWINGS

FIG. 1. Experimental setup for STS-115 Salmonella typhimurium microarray and virulence experiments. This flowchart displays a timeline of how STS experiments were designed and organized. Fluid processing apparatuses (FPAs) were loaded as in FIG. 2 and delivered to Shuttle, activated during spaceflight, and recovered upon landing as outlined in the flowchart. A more detailed description of the FPA activation and fixation/supplementation steps is provided in FIG. 2. OES: Orbital Environmental Simulator (this is a climate-controlled room at Kennedy Space Center that houses ground controls and is maintained at the same temperature and humidity as the Space Shuttle via real-time communications). STS: Space Transport System, refers here to the Shuttle. SLSL: Space Life Sciences Lab.

FIG. 2A-2C. Diagram and photographs of fluid processing apparatuses (FPAs) used in the STS experiments. Panel 2A: Schematic diagram of an FPA. An FPA consists of a glass barrel that contains a short bevel on one side and stoppers inside that separate individual chambers containing fluids used in the experiment. The glass barrel loaded with stoppers and fluids is housed inside a lexan sheath containing a plunger that pushes on the top stopper to facilitate mixing of fluids at the bevel. The bottom stopper in the glass barrel (and also the bottom of the lexan sheath) is designed to contain a gas-permeable membrane that allows air exchange during bacterial growth. In the STS experiments, the bottom chamber contained media, the middle chamber contained the bacterial inoculum suspended in PBS (or water for yeast/fungi), and the top chamber contained either RNA/protein fixative or additional media. Upon activation, the plunger was pushed down so that only the middle chamber fluid was mixed with the bottom chamber to allow media inoculation and bacterial growth. At this step, the plunger was pushed until the bottom of the middle rubber stopper was at the top part of the bevel. After the 25-hour growth period, the plunger was pushed until the bottom of the top rubber stopper was at the top part of the bevel such that the top chamber fluid was added. Panel 2B: Photograph of FPAs in pre-flight configuration. Panel 2C: Photograph of FPAs in post-flight configuration showing that all stoppers have been pushed together and the entire fluid sample is in the bottom chamber.

FIGS. 3A-3C. The rotating wall vessel (RWV) bioreactor and power supply. Panel 3A: The cylindrical culture vessel is completely filled with culture medium through ports on the face of the vessel and operates by rotating around a central axis. Cultures are aerated through a hydrophobic membrane that covers the back of the cylinder. The power supply is shown below the bioreactor. Panel 3B: The two operating orientations of the RWV are depicted. In the LSMMG orientation (panel i), the axis of rotation of the RWV is perpendicular to the direction of the gravity force vector. In the normal gravity (or 1×g) orientation (panel ii), the axis of rotation is parallel with the gravity vector. Panel 3C: The effect of RWV rotation on particle suspension is depicted. When the RWV is not rotating, or rotating in the 1×g orientation (panel i), the force of gravity will cause particles in apparatus to sediment and eventually settle on the bottom of the RWV. When the RWV is rotating in the LSMMG position (panel ii), particles are continually suspended in the media. The media within the RWV rotates as a single body, and the sedimentation of the particle due to gravity is offset by the upward forces of rotation. The result is low shear aqueous suspension that is strikingly similar to what would occur in true microgravity, and is also relevant to certain areas in the human body, including those routinely encountered by pathogens—such as GI and urogenital tracts.

FIGS. 4A-4E. Data from STS-115 Salmonella typhimurium experiments. Panel 4A: Map of the 4.8 Mb circular Salmonella typhimurium genome with the locations of the genes belonging to the spaceflight transcriptional stimulon indicated as black hatch marks. Panel 4B: Decreased time-to-death in mice infected with flight S. typhimurium as compared to identical ground controls. Female Balb/c mice perorally infected with 10⁷ bacteria from either spaceflight or ground cultures were monitored every 6-12 hours over a 30 day period and the percent survival of the mice in each group was graphed versus number of days. Panel 4C: Increased percent mortality of mice infected with spaceflight cultures across a range of infection dosages. Groups of mice were infected with increasing dosages of bacteria from spaceflight and ground cultures and monitored for survival over 30 days. The percent mortality (calculated as in (23)) of each dosage group is graphed versus the dosage amount. Panel 4D: Decreased LD₅₀ value (calculated as in (23)) for spaceflight bacteria in murine infection model. Panel 4E: Scanning electron microscopy (3500× magnification) of spaceflight and ground S. typhimurium bacteria showing the formation of an extracellular matrix and associated cellular aggregation of spaceflight cells relevant to biofilm formation.

FIGS. 5A-5B. Hfq is required for S. typhimurium LSMMG-induced phenotypes in RWV culture. Panel 5A: The survival ratio of wild type and isogenic hfq, hfq 3′ Cm, and invA mutant strains in acid stress after RWV culture in the LSMMG and 1×g positions is plotted (ANOVA p-value<0.05). Panel 5B: Fold intracellular replication of S. typhimurium strains hfq 3′Cm and Δhfq in J774 macrophages after RWV culture as above. Intracellular bacteria were quantitated at 2 hours and 24 hours post-infection, and the fold increase in bacterial numbers between those two time periods was calculated (ANOVA p-value<0.05).

FIGS. 6A-6C. Increased virulence of S. typhimurium in response to spaceflight in LB medium is not observed in M9 minimal medium or LB medium supplemented with M9 salts. 6A, Ratio of LD₅₀ values of S. typhimurium spaceflight and ground cultures grown in LB, M9, or LB-M9 salts media. Female Balb/c mice were perorally infected with a range of bacterial doses from either spaceflight or ground cultures and monitored over a 30-day period for survival. 6B, Time-to-death curves of mice infected with spaceflight and ground cultures from STS-115 (infectious dosage: 10⁷ bacteria for both media). 6C, Time-to-death curves of mice infected with spaceflight and ground cultures from STS-123 (infectious dosage: 10⁶ bacteria for LB and 10⁷bacteria for M9 and LB-M9 salts).

FIG. 7. qRT-PCR analysis of S. typhimurium genes altered in response to spaceflight as compared to ground controls in LB and M9 cultures. Total RNA harvested from spaceflight and ground cultures in the indicated media was converted to single-stranded cDNA and used as a template in qRT-PCR analysis with primers hybridizing to the indicated genes. PCR product levels were normalized to the 16S rRNA product and a ratio of each gene level in flight and ground cultures was calculated. All differences in expression between spaceflight and ground cultures were found to be statistically significant using student's t-test (p-value<0.05).

FIG. 8. Altered acid tolerance of S. typhimurium in ground-based spaceflight analog culture is not observed in the presence of increased phosphate ion concentration. Cultures of S. typhimurium grown in the indicated medium in the rotating wall vessel in the low-shear modeled microgravity (LSMMG) or control orientation were subjected to acid stress (pH 3.5) immediately upon removal from the apparatus. A ratio of percent survival of the bacteria cultured at each orientation in each media is presented.

FIG. 9. Microscopic images of cells of a recombinant attenuated Salmonella anti-pneumococcal vaccine strain scraped off of the hydrophobic membranes of the RWV cultured in 1×G or LSMMG conditions.

FIG. 10. Scanning electron microscopy (SEM) shows profound hyphal formation of C. albicans during spaceflight culture—but no hyphal formation is evident during ground culture of identical controls.

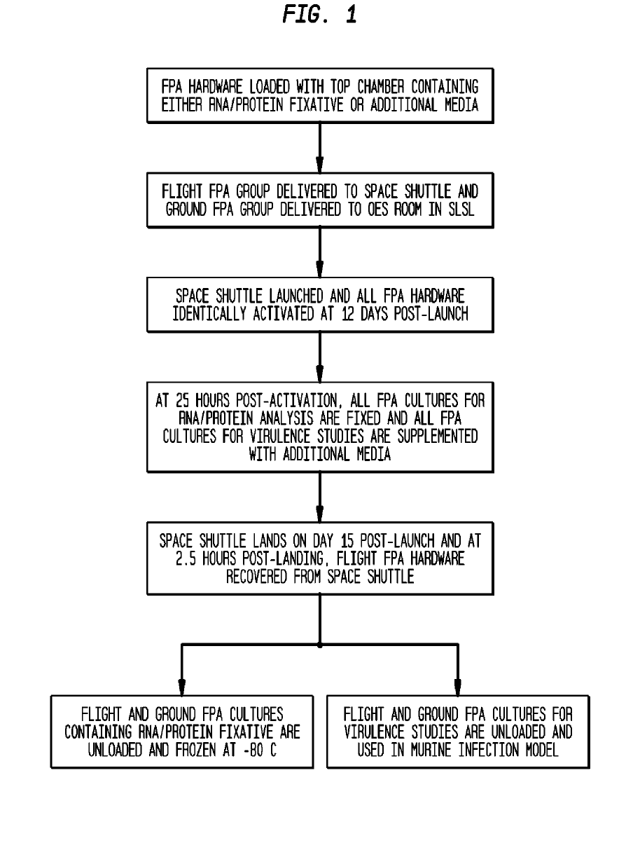

### DETAILED DESCRIPTION OF THE INVENTION

The present invention is predicated in part on the discovery of global changes in microorganisms which resulted from growth in spaceflight or spaceflight analogs which produce low sedimental shear environments around the microorganisms, including phenotypic (such as virulence and stress resistance) and molecular genetic (gene expression) changes. Specifically, during spaceflight aboard the Space Shuttle mission STS-115 and STS-123 and on the ground using a spaceflight analogue bioreactor, the Rotating Wall Vessel, changes were observed in gene expression (mRNA and protein), virulence, and stress resistance using the microorganism Salmonella typhimurium, for example. Further, a conserved global regulator, the Hfq protein, has been identified to be involved in the response to the environment of low sedimental shear stress during spaceflight and spaceflight analogue culture. Conventional culture conditions, which are currently available in the marketplace, do not have the capability to grow microorganisms in low sedimental shear environments, and therefore are unable to recapitulate low fluid shear levels found within an infected host. The recognition of the phenotypic and molecular genetic changes of microorganisms in response to low sedimental shear environments allows the development of modified microorganism with desirable and improved phenotypic characteristics, such as enhanced immunogenicity and protection against infection, altered stress resistance, altered metabolic capabilities, and altered ability to form biofilms. The modified microorganism can be used in formulating therapeutic and vaccine compositions, as well as bioindustrial products. Further, the use of low sedimental shear environments in accordance with the present invention permits identification of novel target molecules for vaccine and therapeutic development, which would not have been possible using conventional culture conditions.

In one aspect, the present invention provides a method for modifying or manipulating a microorganism by culturing the microorganism under low sedimental fluid shear conditions, and harvesting the cultured microorganism. This aspect of the invention excludes culturing a Salmonella sp., particularly a wild type (i.e., naturally occurring, unmodified Salmonella sp.) in a low sedimental fluid shear environment created by a rotating wall vessel bioreactor.

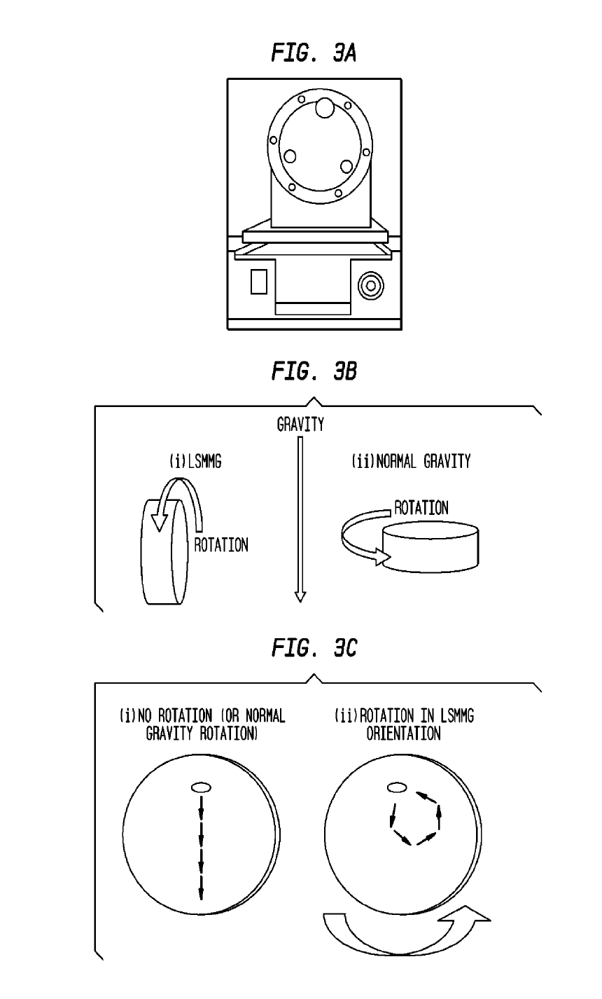

“Low sedimental fluid shear conditions” and “low sedimental fluid shear environments”, or in short, “low sedimental shear” conditions or environments, contemplated by the present invention include space flight and space flight analogs which produce low sedimental shear environments. Examples of space flight analog include commercial analog bioreactors such as rotating wall vessels (RWV), and other art-recognized low sedimental shear environments as understood by the skilled artisan. The RWV is a rotating bioreactor (FIG. 3) in which cells are maintained in suspension in a gentle fluid orbit that creates a sustained low-fluid-shear and microgravity environment. The level of fluid shear force within the bioreactor can be increased in a controlled and quantitative manner by adding beads (e.g., polypropylene beads) of a selected size to the RWV (Nauman et al., Applied and Environmental Microbiology 73: 699-705, 2007). By using beads of different sizes, a range of fluid shear levels can be achieved, which can be relevant to those encountered by a microorganism in an infected host. For example, fluid shear levels in the RWV can be adjusted from lower than 0.01 dynes per cm² in the absence of beads, to 5.2 dynes per cm² by adding 3/32-inch beads, to 7.8 dynes per cm² by adding ⅛-inch beads, as determined and described by Nauman et al. (2007), incorporated herein in its entirety by reference. According to the present invention, fluid shear levels of 100 dynes per cm² or lower, preferably lower than 50 dynes per cm², more preferably lower than 20 dynes per cm², even more preferably 10 dynes per cm² or lower, or even lower than 0.1 dynes per cm², are considered low shear levels.

The term “microorganism” includes bacteria, viruses, fungi, protozoa, protists, and worms (such as helminthes), among others. Examples of microorganisms contemplated by the present invention include Salmonella sp. (particularly Salmonella typhimurium), Streptococcus pneumoniae, Pseudomonas aeruginosa, Candida albicans, and Saccharomyces cerevisiae.

In one embodiment, modification of the microorganism is achieved by altering the fluid shear levels in the low sedimental shear environment in which the microorganism is being cultured. The fluid shear level in the culture can be adjusted to 100 dynes per cm² or lower, preferably lower than 50 dynes per cm², more preferably lower than 20 dynes per cm², even more preferably 10 dynes per cm² or lower, or even lower than 0.1 dynes per cm².

In accordance with the present invention, culturing a microorganism in low sedimental shear environments induces both phenotypic and molecular genetic changes. Accordingly, in a further aspect, the present invention provides methods of modifying a phenotypic characteristic of a microorganism by culturing the microorganism in low sedimental shear environments.

A “phenotypic characteristic” of a microorganism, as would be understood by those skilled in the art, include any observable or detectable physical or biochemical characteristics of a microorganism, including but not limited to, virulence, immunogenicity, stress resistance (such as thermal, acid or oxidative stress resistance), resistance to drugs including anti-microbial compounds (e.g., resistance of a fungus to an anti-fungal compound), ability of a bacterium to form biofilm in culture, among others.

Changes in phenotypic characteristics are often associated with or caused by molecular genetic changes. As used herein, the term “molecular genetic changes” refer to changes in gene expression, which manifest at any or a combination of mRNA, rRNA, tRNA, small non-coding RNA levels and protein levels.

The present inventors have demonstrated global changes in gene expression, virulence and stress resistance characteristics of Salmonella typhimurium, which resulted from growth in a spaceflight or spaceflight analog (RWV) which produces low sedimental shear environments around the cells. A conserved global regulator, the Hfq protein, has been identified to be involved in the response to the environment of low sedimental shear stress during spaceflight and spaceflight analogue culture. While Salmonella typhimurium has been used as an example to illustrate the phenotypic and genetic changes, due to the nature of the effect and the conservation of global regulators between different organisms, multiple organisms should display similar changes in characteristics in response to low sedimental shear environments.

In one embodiment, low sedimental shear conditions are applied to a microorganism to alter (increase or decrease) the virulence of the microorganism. In a specific embodiment, low sedimental shear conditions are applied to a microorganism to increase the virulence of the microorganism. By “virulence” it is meant the ability of a microorganism to cause disease. Virulence of a microorganism can be determined by any of the art-recognized methods, including suitable animal models. The ability of low sedimental shear conditions to increase virulence of a microorganism allows for the development of new therapeutic compositions. Without being bound to any particular theory, the global changes of a microorganism resulting from culturing in a low sedimental shear environment may include expression of antigens by the microorganism that would not be expressed under conventional culturing conditions but are possibly expressed during infection of a host by the microorganism. In addition, a microorganism exhibits enhanced stress resistance and improved ability of survival after being cultured in a low sedimental shear environment. As a result, a vaccine prepared using such microorganism is able to survive longer in a recipient host to induce desirable protective immunity.

In one embodiment, low sedimental shear conditions are applied to an attenuated vaccine strain of microorganism to enhance the efficacy of the vaccine strain. Enhanced vaccine efficacy includes, but is not limited to improved immunogenicity (i.e. ability of the vaccine strain to provoke immune response), and/or improved protection against subsequent challenges. Attenuated microbial vaccine strains are well-documented in the art and can be prepared by various well-known methods, such as serial passaging or site-directed mutagenesis.

In a specific embodiment, the present invention provides a method of enhancing the immunogenicity and/or protection of an attenuated Salmonella vaccine strain by culturing the attenuated Salmonella vaccine strain in a low sedimental shear environment and harvesting the cultured strain.

In another specific embodiment, the present invention provides a method of enhancing the immunogenicity and/or protection of a recombinant attenuated Salmonella vaccine strain expressing one or more antigens from other pathogens by culturing the attenuated recombinant Salmonella vaccine strain in a low sedimental shear environment and harvesting the cultured strain.

In another embodiment, low sedimental shear conditions are applied to a microorganism to enhance stress resistance of the microorganism. The resulting, more resilient microorganism is particularly useful for the development of biomedical products like vaccines and bioindustrial products, such as biofuels. Enhanced performance and robustness of consortia of microorganisms are also useful for bioremediation.

In still another embodiment, low sedimental shear conditions are applied to a microorganism to modify the ability of the microorganism to form biofilm. In a specific embodiment, low sedimental shear conditions are applied to a microorganism to enhance the ability of the microorganism to form biofilm. As illustrated hereinbelow, S. typhimurium strain X3339, which does not form biofilm when cultured in the LB medium in ground, is able to form biofilm after grown in spaceflight. On the other hand, an attenuated S. typhimurium strain vaccine strain X9558pYA4088, which forms biofilm in 1×g culture in the RWV, showed reduced ability to form biofilm after grown in LSMMG. Accordingly, one could employ low sedimental shear conditions to manipulate the ability of the microorganism to form biofilm, either to increase such ability in order to develop bioindustrial products useful for sewage treatment and pollution control, or to decrease the ability to form biofilm. Altered biofilm production could be important for enhanced efficacy and robustness of microbial consortia for bioremediation, sewage treatment, microbial fuel cells, and possibly vaccines.

In a further aspect, the present invention is directed to the modulation of one or more ion concentrations to manipulate, e.g., to amplify or inhibit, responses of microorganisms to low sedimental shear environments.

The present inventors have discovered that the environmental ion concentration during microbial growth strongly influences the intensity of changes in virulence and gene expression profiles in response to low sedimental shear conditions. For example, higher concentrations of phosphate ions altered the ability of S. typhimurium to respond to spaceflight and minimized its pathogenic-related effects. The term “ions” as used herein is not limited to one particular type of ion, and includes, e.g., phosphate, chloride, sulfate/sulfur, bromide, nitrate-n, o-phosphate, pH/hydrogen ion, calcium, chromium, copper, iron, lithium, fluoride, magnesium, manganese, molybdenum, nickel, potassium, sodium and zinc, among others.

In one embodiment, one or more ion concentrations are modulated to inhibit pathogenic responses of microorganisms to low sedimental shear environments. Such modulations are useful in human spaceflight to mitigate the adverse effects of microorganisms necessarily present and undergoing the subject pathogenic responses during and because of such flight. Such modulations are also useful to counteract pathogenic responses of microorganisms to low sedimental shear environments encountered during infection of a host, in which case, modulation of ion concentrations can be achieved by oral administration to the host with compositions containing one or more ions, or ion chelators.

In one embodiment, one or more ion concentrations are modulated to modulate, i.e., to amplify or decrease, the responses of microorganisms to low sedimental shear environments. Such modulations are useful, e.g., to enhance the immunogenicity of a strain for the development of vaccine or other therapeutic products, to enhance the stress resistance of a microorganism for the development of bioindustrial products.

In a further aspect, the present invention provides microorganisms harvested from a low sedimental shear culture.

In one embodiment, the present invention provides Salmonella sp. obtained from a culture grown in spaceflight. In a preferred embodiment the microorganism is Salmonella typhimurium. Salmonella sp., particularly wild type (native) Salmonella sp., obtained from a culture grown under low sedimental shear conditions provided by the RWVs is excluded from the scope of the present invention. In another embodiment, the present invention provides Streptococcus pneumonia harvested from a culture grown under low sedimental shear conditions. In still another embodiment, the present invention provides Pseudomonas aeruginosa harvested from a culture grown under low sedimental shear conditions. In yet another embodiment, the present invention provides a fungus, such as Candida albicans and Saccharomyces cerevisiae, harvested from a culture grown under low sedimental shear conditions.

In another aspect, the present invention provides a therapeutic composition comprised of a microorganism obtained from a low sedimental shear culture. The therapeutic composition can be a vaccine composition with improved efficacy as compared to a vaccine made of the same microorganism grown in a control (normal) sedimental shear culture. In one embodiment, the present invention provides a vaccine composition containing Salmonella sp. obtained from a culture grown under low sedimental shear conditions. In a preferred embodiment the microorganism is Salmonella typhimurium. In another preferred embodiment, the present invention provides a vaccine composition containing a recombinant attenuated Salmonella anti-pneumococcal vaccine strain harvested from a culture grown under low sedimental shear conditions. In another embodiment, the present invention provides a vaccine containing Streptococcus pneumonia harvested from a culture grown under low sedimental shear conditions. In still another embodiment, the present invention provides a vaccine containing Pseudomonas aeruginosa harvested from a culture grown under low sedimental shear conditions. In yet another embodiment, the present invention provides a therapeutic composition containing a fungus, such as Candida albicans and Saccharomyces cerevisiae, harvested from a culture grown under low sedimental shear conditions.

Other compositions formulated with a microorganism obtained from a low sedimental shear culture, useful for various bioindustrial applications, are also included within the scope of the present invention.

In a further aspect, the present invention provides a method for identifying a gene of a microorganism which modulates the response of the microorganism to low sedimental shear environments.

Conventional culture conditions, which are currently available in the marketplace, do not have the capability to grow microorganisms in low sedimental shear environments, and therefore are unable to recapitulate low fluid shear levels found within an infected host. Thus, many of the genes that could be expressed or proteins that could be functional are not documented or investigated. These genes are critical to understanding microbial responses during growth in many unique conditions, such as spaceflight, and in many common conditions encountered during the course of microbial natural lifecycles, such as locations in the host during microbial infection. Low sedimental shear environments are useful to identify classes of genes (including regulatory RNAs) and proteins that have heretofore not been recognized, characterized or understood from microorganisms cultured in standard culture conditions.

As demonstrated by the present inventors, the space-traveling Salmonella had changed expression of 167 genes, as compared to bacteria that remained on Earth. A conserved global regulator, the Hfq protein, has been identified to be involved in the response to the environment of low sedimental shear stress during spaceflight and spaceflight analogue culture. Bacteria that lack the

Hfq gene did not respond to the low sedimental shear conditions. These results highlight Hfq as a therapeutic target. In addition, a number of genes have been identified in accordance with the present invention to respond in the same direction in both RWV microarray analysis and spaceflight analysis, including dps, fimA, hfq, ptsH, rplD, and yaiV. Several genes have been identified to be regulated in different directions in the two conditions (i.e. up in RWV, but down in flight or vice versa), including ppiB, sipD and frdC.

According to the present invention, microbial genes that modulate the response of a microorganism to low sedimental shear environments can be identified by culturing the microorganism in a low sedimental shear environment, and comparing expression (at mRNA or protein level) of candidate genes in the microorganism in the low sedimental shear environment relative to ground control conditions. Those that exhibit differential expression can be identified from candidate genes. The embodiment of identifying modulator genes by culturing a Salmonella species in a low sedimental shear environment provided by the RWV is excluded from this aspect of the invention.

Gene expression can be determined by a variety of art-recognized techniques, including but not limited to, microarray analysis of mRNA, rRNA, tRNA, or small non-coding RNA, RT-PCR or qRT-PCR, Western blot, and proteomics analysis. By “differential expression” it is meant that the ratio of the levels of expression under two different conditions is at least 1.5, preferably at least 2.0, more preferably at least 3.0, even more preferably 5.0 or more. After microorganisms are harvested from a spaceflight or spaceflight analog (such as the RWV bioreactor described above), cells are processed so as to retain the expression profile from a low sedimental shear culture, prior to a specific target identification assay is being performed. For example, for microarray analysis, cells are fixed immediately in RNA Later™ or other relevant fixative. Total RNA is isolated from cells, labeled with fluorescent dyes (such as Cy3 and Cy 5), and used to hybridize to microarrays with genomic DNA. Two assays are performed, one for LSS (low sedimental shear) and one for CSS (control sedimental shear) cultured cells, respectively. After quantitation, the ratio of expression of LSS to CSS is determined. Genes with ratios of 2 or greater (or 0.5 or less) (either up or down-regulated in LSS, respectively) can be identified, for example. For proteomics, cells are fixed using RNA Later or similar fixative, or fixed by flash freezing and storage at −80 degrees C. Cells are lysed and proteins are precipitated with acetone. After digestion with trypsin, the protein samples are subjected to a proteomic assay of choice: MudPIT, LC/MS-MS, 2-D gels followed by MALDI, for example. Proteins that are present under LSS conditions and not in CSS (or vice-versa) can be identified. For Western blotting, cells are fixed using RNA Later or similar fixative, or by flash freezing and storage at −80 degrees C. Fixed cells are resuspended in a protein sample buffer for SDS-PAGE and run on gel, followed by Western blot analysis using antisera from patients/animals or immunizations against prominent antigens. Prominent proteins bands in LSS samples as compared to CSS samples will correspond to proteins that are recognized by the patient/animal and up-regulated under LSS conditions. Protein bands can be cut out and are subjected to MALDI to identify the molecular nature of the underlying protein(s).

Functional categories of genes that have been or can be identified as differentially expressed in accordance with the present invention include, without limitation, virulence genes, iron metabolism genes, ion response or utilization genes, cell surface polysaccharide genes, protein secretion genes, flagellar genes, stress genes, genes coding for ribosomal proteins, genes coding for fimbrial proteins, transcriptional regulator genes, genes involved in extracellular matrix/biofilm synthesis, stress response genes, sigma factors, genes encoding RNA binding proteins, genes encoding small noncoding regulatory RNAs (small RNAs), DNA polymerase genes, RNA polymerase genes, plasmid transfer/conjugation genes, chaperone proteins, carbon utilization genes, Metabolic pathway genes, energy metabolism genes, chemotaxis genes, genes encoding heat shock proteins, genes encoding putative proteins, genes encoding recombination proteins, genes encoding transport system proteins, genes encoding membrane proteins, genes encoding cell wall components (including LPS), housekeeping genes, genes encoding structural proteins and enzymes, and plasmid genes.

In certain instances, the functions of identified genes may have been already documented. In other cases, the functions are unknown, or their unique expression in LSS conditions is unknown. The functions of differentially expressed genes identified from LSS cultures can be further characterized by making a mutant microorganism in which a particular gene of interest is mutated (e.g., completely knocked out), and assessing whether the mutant microorganism exhibits any change in virulence, stress resistance or any other phenotypic characteristics, and therefore determining whether this gene is involved in establishing infection, for example. Alternatively, the expression of a gene of interest, which has been identified from LSS cultures, can be altered by mutating its promoter, or completing replacing its promoter with a heterologous promoter, to increase or decrease its expression in order to determine the role of the gene in establishing infection.

If a gene differentially expressed in LSS conditions is determined to be involved in establishing infection, such gene makes a good target, because a loss of function in this gene will be expected to decrease the ability of the microorganism to cause infection. This will provide a basis for intelligent design of pharmaceutical compounds for treating and preventing infection by this microorganism.

Further, proteins identified as uniquely expressed in LSS conditions can be used as antigen for immunizations.

In still a further aspect of the present invention, LSS cultures are used for screening for new drugs against infection by a microorganism. This is achieved by culturing the microorganism in a LSS environment, contacting the microorganism in the culture with a candidate compound, and determining the inhibitory effect of the compound on the growth of the microorganism as indicative of the therapeutic efficacy of the compound. This method of the present invention has the advantage to be able to select compounds that are effective against the microorganism in an in vivo LSS environment during infection.

In another aspect, the present invention is directed to the use of a host, including during space flight, to study interactions between the host and a microorganism pathogen or an attenuated vaccine strain when both are simultaneously placed in a low sedimental shear environment. The pathogen can, for this purpose, also have been manipulated in an RWV or similar analog.

According to this aspect of the invention, hosts include animals, animal analogs, plants, insects, and cell and/or tissue cultures from animals, animal analogs or plants.

In one embodiment, the invention is directed to the use of animal models, including during space flight, as hosts to study interactions between the host and a microorganism pathogen in a low sedimental shear environment. Animal models include those typically used by the art, and include without limitation, animals of the class Mammalia; preferably rodents such as mice, rats and the like.

In another embodiment, the present invention is directed to the use of so-called animal model analogs as hosts, including during spaceflight, to examine the host-pathogen interaction. Animal model analogs as hosts include those known in the art, such as without limitation, invertebrates, e.g. from the class Nematoda and the like, for the purpose herein.

In still another embodiment, the invention contemplates the use of plants as hosts, including during space flight, to examine the effect of space flight on the host-pathogen interaction, e.g., that leads to infection and disease.

In yet another embodiment, the invention is directed to the use of cell and/or tissue cultures from animals (including mammals), animal analogs (e.g. invertebrates such as nematodes and the like) and/or plants as hosts, including during space flight, to examine the effect of space flight on the host-pathogen interaction.

This invention is further illustrated by the following examples, which are not to be construed in any way as imposing limitations upon the scope thereof. All the publications mentioned in the present disclosure are incorporated herein by reference.

### Example 1

### Spaceflight Alters Bacterial Gene Expression and Virulence and Reveals Role for Global Regulator Hfq

This example describes experiments conducted with the bacterial pathogen Salmonella typhimurium which was grown aboard Space Shuttle mission STS-115 and compared to identical ground control cultures. Global microarray and proteomic analyses revealed 167 transcripts and 73 proteins changed expression with the conserved RNA-binding protein Hfq identified as a likely global regulator involved in the response to the spaceflight environment. Hfq involvement was confirmed with a ground based microgravity culture model. Spaceflight samples exhibited enhanced virulence in a murine infection model and extracellular matrix accumulation consistent with a biofilm.

### Materials and Methods

### Strains, Media, and Chemical Reagents

The virulent, mouse-passaged Salmonella typhimurium derivative of SL1344 termed χ3339 was used as the wild type strain in all flight and ground-based experiments (5). Isogenic derivatives of SL1344 with mutations Δhfq, hfq 3′Cm, and invA Km were used in ground-based experiments (13, 22). The Δhfq strain contains a deletion of the hfq open reading frame (ORF) and replacement with a chloramphenicol resistance cassette, and the hfq 3′Cm strain contains an insertion of the same cassette immediately downstream of the WT hfq ORF. The invA Km strain contains a kanamycin resistance cassette inserted in the invA ORF. Lennox broth (LB) was used as the growth media in all experiments (5) and phosphate buffered saline (PBS) (Invitrogen, Carlsbad, Calif.) was used to resuspend bacteria for use as inoculum in the FPAs. The RNA fixative RNA Later II (Ambion, Austin, Tex.), glutaraldehyde (16%) (Sigma, St. Louis, Mo.), and formaldehyde (2%) (Ted Pella Inc., Redding, Calif.) were used as fixatives in flight experiments.

### Loading of Fluid Processing Apparatus (FPA)

An FPA consists of a glass barrel that can be divided into compartments via the insertion of rubber stoppers and a lexan sheath into which the glass barrel is inserted. Each compartment in the glass barrel was filled with a solution in an order such that the solutions would be mixed at specific timepoints in flight via two actions: (1) downward plunging action on the rubber stoppers and (2) passage of the fluid in a given compartment through a bevel on the side of the glass barrel such that it was released into the compartment below. Glass barrels and rubber stoppers were coated with a silicone lubricant (Sigmacote, Sigma, St. Louis, Mo.) and autoclaved separately before assembly. A stopper with a gas exchange membrane was inserted just below the bevel in the glass barrel before autoclaving. FPA assembly was performed aseptically in a laminar flow hood in the following order: 2.0 ml LB media on top of the gas exchange stopper, one rubber stopper, 0.5 ml PBS containing bacterial inoculum (approximately 6.7×10⁶ bacteria), another rubber stopper, 2.5 ml of either RNA fixative or LB media, and a final rubber stopper. Syringe needles (gauge 25⅝) were inserted into rubber stoppers during this process to release air pressure and facilitate assembly. To facilitate group activation of FPAs during flight and to ensure proper containment levels, sets of 8 FPAs were loaded into larger containers termed group activation packs (GAPs).

### Murine Infection Assay

Six to eight week old female Balb/c mice (housed in the Animal Facility at the Space Life Sciences Lab at Kennedy Space Center) were fasted for approximately 6 hours and then per-orally infected with increasing dosages of S. typhimurium harvested from flight and ground FPA cultures and resuspended in buffered saline gelatin (5). Ten mice per infectious dosage were used, and food and water were returned to the animals within 30 minutes post-infection. The infected mice were monitored every 6-12 hours for 30 days. The LD₅₀ value was calculated using the formula of Reed and Muench (23).

### Scanning Electron Microscopy

A portion of cells from the viable, media-supplemented cultures from flight and ground FPAs were fixed for scanning electron microscopic analysis using 8% glutaraldehyde and 1% formaldehyde and were processed for SEM as described previously (24).

### Microarray Analysis

Total cellular RNA purification, preparation of fluorescently-labeled, single stranded cDNA probes, probe hybridization to whole genome S. typhimurium microarrays, and image acquisition was performed as previously described (7) using three biological and three technical replicates for each culture condition. Flow cytometric analysis revealed that cell numbers in flight and ground biological replicate cultures were not statistically different (using SYTO-BC dye per manufacturer's recommendations; Invitrogen, Carlsbad, Calif.). Data from stored array images were obtained via QuantArray software (Packard Bioscience, Billerica, Mass.) and statistically analyzed for significant gene expression differences using the Webarray suite as described previously (25). GeneSpring software was also used to validate the genes identified with the Webarray suite. To obtain the genes comprising the spaceflight stimulon as listed in Table 1, the following parameters were used in Webarray: a fold increase or decrease in expression of 2 fold or greater, a spot quality (A-value) of greater than 9.5, and p-value of less than 0.05. For some genes listed in Table 1, the following parameters were used: a fold increase or decrease in expression of value greater than 1.6 or less then 0.6 respectively, an A-value of 8.5 or greater, and p-value of less then 0.1. The vast majority of genes listed in Table 1 had an A-value of greater than 9.0 (with most being greater than 9.5) and a p-value of 0.05 or less. The exceptions were as follows: sbmA (p-value=0.06), oxyS (A-value=8.81), rplY (A-value=8.95), cspD (A-value=8.90), yfiA (p-value=0.08), ompX (p-value=0.09), has (p-value=0.08), rmf (A-value=8.82), wcaD (p-value 0.09), and fliE (A-value=8.98). To identify spaceflight stimulon genes also contained in the Hfq regulon, proteins or genes found to be regulated by Hfq or RNAs found to be bound by Hfq as reported in the indicated references were scanned against the spaceflight microarray data for expression changes within the parameters above (8, 12, 13, 16, 26).

### Multidimensional Protein Identification (MudPIT) Analysis Via Tandem Mass Spectrometry Coupled to Dual Nano-Liquid Chromatography (LC-LC-MS/MS)

Acetone-protein precipitates from whole cell lysates obtained from flight and ground cultures (representing three biological replicates) were subjected to MudPIT analysis using the LC-LC-MS/MS technique as described previously (27, 28). Tandem MS spectra of peptides were analyzed with TurboSEQUEST™ v 3.1 and XTandem software, and the data were further analyzed and organized using the Scaffold program (29, 30). See Table 2 for the specific parameters used in Scaffold to identify the proteins in this study.

### Ground-Based RWV Cultures and Acid Stress and Macrophage Survival Assays

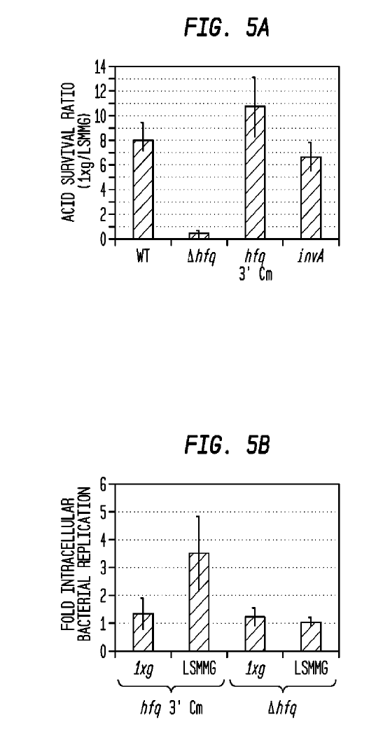

S. typhimurium cultures were grown in rotating wall vessels in the LSMMG and 1×g orientations and assayed for resistance to pH=3.5 and survival inside J774 macrophages as described previously (5), except that the RWV cultures were grown for 24 hours at 37 degrees C. For acid stress assays, the percentage of surviving bacteria present after 45-60 minutes acid stress (compared to the original number of bacteria before addition of the stress) was calculated. A ratio of the percent survival values for the LSMMG and 1×g cultures was obtained (indicating the fold difference in survival between these cultures) and is presented as the acid survival ratio in FIG. 5A. The mean and standard deviation from three independent experimental trials is presented. For macrophage survival assays, the number of bacteria present inside J774 macrophages at 2 hours and 24 hours post-infection was determined, and the fold difference between these two numbers was calculated. The mean and standard deviation of values from three independent experimental trials (each done in triplicate tissue culture wells) is presented. The statistical differences observed in the graphs in FIG. 5 were calculated at p-values less than 0.05.

### Results

### Whole-Genome Transcriptional and Proteomic Analysis of Spaceflight and Ground Cultures.

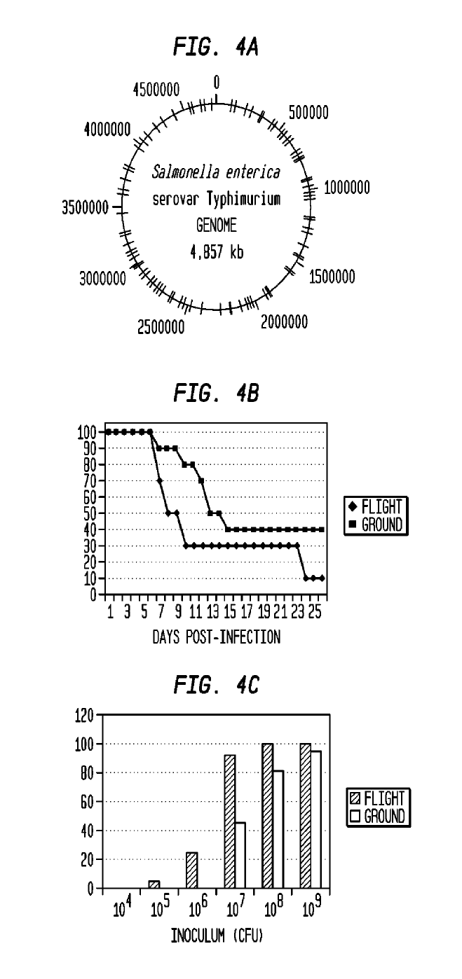

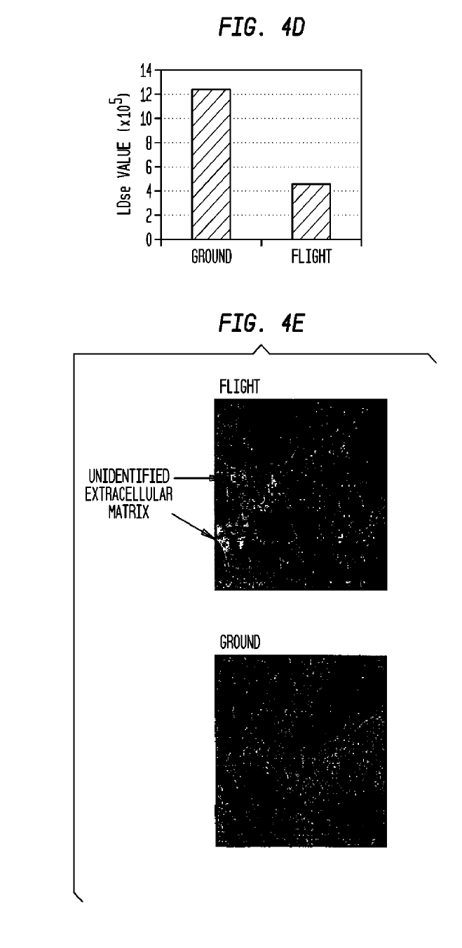

To determine which genes changed expression in response to spaceflight, total bacterial RNA was isolated from the fixed flight and ground samples, qualitatively analyzed to ensure lack of degradation via denaturing gel electrophoresis, quantitated, and then reversed transcribed into labeled, single-stranded cDNA. The labeled cDNA was co-hybridized with differentially-labeled S. typhimurium genomic DNA to whole genome S. typhimurium microarray slides. The cDNA signal hybridizing to each gene spot was quantitated, and the normalized, background-subtracted data was analyzed for statistically-significant, 2-fold or greater differences in gene expression between the flight and ground samples. 167 genes were found to be differentially-expressed in flight as compared to ground controls from a variety of functional categories (69 up-regulated and 98 down-regulated) (Table 1). The proteomes of fixed cultures were also obtained via multi-dimensional protein identification (MudPIT) analysis. Among 251 proteins expressed in the flight and ground cultures, 73 were present at different levels in these samples (Table 2). Several of the genes encoding these proteins were also identified via microarray analysis. Collectively, these gene expression changes form the first documented bacterial spaceflight stimulon indicating that bacteria respond to this environment with widespread alterations of expression of genes distributed globally throughout the chromosome (FIG. 4, Panel A).

### Involvement of Hfq in Spaceflight and LSMMG Responses

The data indicated that a pathway involving the conserved RNA-binding regulatory protein Hfq played a role in this response (Table 3). Hfq is an RNA chaperone that binds to small regulatory RNA and mRNA molecules to facilitate mRNA translational regulation in response to envelope stress (in conjunction with the specialized sigma factor RpoE), environmental stress (via alteration of RpoS expression), and changes in metabolite concentrations, such as iron levels (via the Fur pathway) (8-12). Hfq is also involved in promoting the virulence of several pathogens including S. typhimurium (13), and Hfq homologues are highly conserved across species of prokaryotes and eukaryotes (14). The data strongly supported a role for Hfq in the response to spaceflight: (1) The expression of hfq was decreased in flight, and this finding matched previous results in which S. typhimurium hfq gene expression was decreased in a ground-based model of microgravity (7); (2) Expression of 64 genes in the Hfq regulon was altered in flight (32% of the total genes identified), and the directions of differential changes of major classes of these genes matched predictions associated with decreased hfq expression (see subsequent examples); (3) several small regulatory RNAs that interact with Hfq were differentially regulated in flight as would be predicted if small RNA/Hfq pathways are involved in a spaceflight response; (4) The levels of OmpA, OmpC, and OmpD mRNA and protein are classic indicators of the RpoE-mediated periplasmic stress response which involves Hfq (15). Transcripts encoding OmpA, OmpC, and OmpD (and OmpC protein level) were up-regulated in flight, correlating with hfq down-regulation; (5) Hfq promotes expression of a large class of ribosomal structural protein genes (12), and many such genes exhibited decreased expression in flight; (6) Hfq is a negative regulator of the large tra operon encoding the F plasmid transfer apparatus (16), and several tra genes from related operons on two plasmids present in S. typhimurium χ3339 were up regulated in flight; (7) Hfq is intimately involved in a periplasmic stress signaling pathway that is dependent on the activity levels of three key proteins, RpoE, DksA, and RseB: differential expression of these genes was observed in flight (8, 12); (8) Hfq regulates the expression of the Fur protein and other genes involved in the iron response pathway, and several iron utilization/storage genes were found to have altered expression in flight (9, 11). This finding also matched previous results in which iron pathway genes in S. typhimurium changed expression in a ground-based model of microgravity, and the Fur protein was shown to play a role in stress resistance alterations induced in the same model (7).

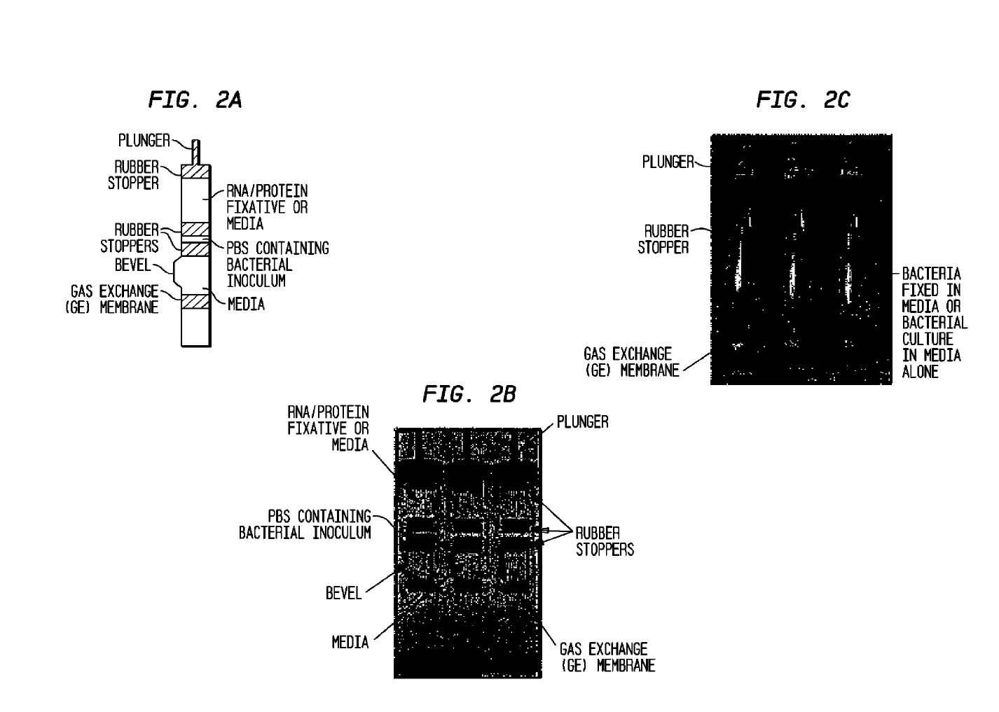

Experiments were performed to verify a role for Hfq in the spaceflight response using a cellular growth apparatus that serves as a ground-based model of microgravity conditions termed the rotating wall vessel (RWV) bioreactor (FIG. 2). Designed by NASA, the RWV has been extensively used in this capacity to study the effects of a biomedically relevant low fluid shear growth environment (which closely models the liquid growth environment encountered by cells in the microgravity environment of spaceflight as well as by pathogens during infection of the host) on various types of cells (6, 17-19). Studies with the RWV involve using two separate apparatus: one is operated in the modeled microgravity position (termed low-shear modeled microgravity or LSMMG) and one is operated as a control in a position (termed 1×g) where sedimentation due to gravity is not offset by the rotating action of the vessel. LSMMG-induced alterations in acid stress resistance and macrophage survival of S. typhimurium have previously been shown to be associated with global changes in gene expression and virulence (5, 7).

Wild type and isogenic hfq mutant strains of S. typhimurium were grown in the RWV in the LSMMG and 1×g positions and assayed for the acid stress response and macrophage survival. While the wild type strain displayed a significant difference in acid resistance between the LSMMG and 1×g cultures, this response was not observed in the hfq mutant, which contains a deletion of the hfq gene and replacement with a Cm-r cassette (FIG. 5, Panel A). Two control strains, hfq 3′Cm (containing an insertion of the Cm-r cassette just downstream of the WT hfq gene) and invA Km (containing a Km-r insertion in a gene unrelated to stress resistance), gave the same result as the WT strain. Intracellular replication of the LSMMG-grown WT (hfq 3′Cm) strain in infected J774 macrophages was also increased as compared to the 1×g control, and this phenotype was not observed in the hfq mutant strain (FIG. 5, Panel B). Collectively, these data indicate that Hfq is involved in the bacterial spaceflight response as confirmed in a ground-based model of microgravity conditions. In addition, the intracellular replication phenotype inside macrophages correlates with the finding that spaceflight and LSMMG cultures exhibit increased virulence in mice (see text below).

Increased Virulence of S. typhimurium Grown in Spaceflight as Compared to Ground Controls.

Since growth during spaceflight and potential global reprogramming of gene expression in response to this environment could alter the virulence of a pathogen, we compared the virulence of S. typhimurium spaceflight samples to identical ground controls as a second major part of our study. Bacteria from flight and ground cultures were harvested and immediately used to inoculate female Balb/c mice via a per-oral route of infection on the same day as Shuttle landing. Two sets of mice were infected at increasing dosages of either flight or ground cultures, and the health of the mice was monitored every 6-12 hours for 30 days. Mice infected with bacteria from the flight cultures displayed a decreased time to death (at the 10⁷ dosage), increased percent mortality at each infection dosage and a decreased LD₅₀ value compared to those infected with ground controls (FIG. 4, Panels B,C,D). These data indicate increased virulence for spaceflight S. typhimurium samples and are consistent with previous studies in which the same strain of S. typhimurium grown in the RWV under LSMMG conditions displayed enhanced virulence in a murine model as compared to 1×g controls (5).

### Scanning Electron Microscopy of Spaceflight and Ground Cultures.

To determine any morphological differences between flight and ground cultures, scanning electron microscopic (SEM) analysis of bacteria from these samples was performed. While no difference in the size and shape of individual cells in both cultures was apparent, the flight samples demonstrated clear differences in cellular aggregation and clumping that was associated with the formation of an extracellular matrix (FIG. 4, Panel E). Consistent with this finding, several genes associated with surface alterations related to biofilm formation changed expression in flight (up-regulation of wca/wza colonic acid synthesis operon, ompA, fimII; down-regulation of motility genes) (Table 3). SEM images of other bacterial biofilms show a similar matrix accumulation (20, 21). Since extracellular matrix/biofilm formation can help to increase survival of bacteria under various conditions, this phenotype indicates a change in bacterial community potentially related to the increased virulence of the flight bacteria in the murine model.

TABLE 1
Salmonella typhimurium genes differentially-regulated during spaceflight mission STS-115

| | | | |
|---|---|---|---|
| Gene | Fold | Gene | |
| number | change | name | Known or putative function |

UP-regulated

| | | | |
|---|---|---|---|
| Secreted proteins: | | | |
| STM1959 (SEQ ID NO: 1) | 2.10 | fliC | flagellar biosynthesis; flagellin, filament |
| | | | structural |
| | | | protein |
| STM2066 (SEQ ID NO: 2) | 2.31 | sopA | Secreted effector protein of Salmonella dublin |
| STM2883 (SEQ ID NO: 3) | 2.57 | sipD | cell invasion protein |
| STM2884 (SEQ ID NO: 4) | 6.28 | sipC | cell invasion protein |
| Membrane proteins: | | | |
| STM0374 (SEQ ID NO: 5) | 2.04 | yaiV | putative inner membrane protein |
| STM1070 (SEQ ID NO: 6) | 2.05 | ompA | putative hydrogenase, membrane component |
| STM1572 (SEQ ID NO: 7) | 3.34 | ompD | outer membrane protein; bacterial porin |
| STM2267 (SEQ ID NO: 8) | 2.44 | ompC | outer membrane protein 1b (ib; c), porin |
| STM3420 (SEQ ID NO: 9) | 3.12 | secY | preprotein translocase of IISP family, putative |
| | | | membrane ATPase |
| Other function: | | | |
| STM0152 (SEQ ID NO: 10) | 2.18 | aceE | pyruvate dehydrogenase, decarboxylase |
| | | | component |
| STM0182 (SEQ ID NO: 11) | 2.21 | panB | 3-methyl-2-oxobutanoate |
| | | | hydroxymethyltransferase |
| STM0240 (SEQ ID NO: 12) | 2.02 | yaeJ | putative-tRNA hydrolase domain |
| STM0272 (SEQ ID NO: 13) | 2.36 | | putative ATPase with chaperone activity; |
| | | | homologue of Yersinia clpB |
| STM0596 (SEQ ID NO: 14) | 2.24 | entE | 2,3-dihydroxybenzoate-AMP ligase |
| STM0730 (SEQ ID NO: 15) | 4.75 | gltA | citrate synthase |
| STM1040 (SEQ ID NO: 16) | 2.12 | | Gifsy-2 prophage; probable minor tail protein |
| STM1290 (SEQ ID NO: 17) | 7.67 | gapA | glyceraldehyde-3-phosphate dehydrogenase A |
| STM1749 (SEQ ID NO: 18) | 4.76 | adhE | iron-dependent alcohol dehydrogenase of AdhE |
| STM2106 (SEQ ID NO: 19) | 2.07 | wcaI | putative glycosyl transferase in colanic acid |
| | | | biosynthesis |
| STM2118 (SEQ ID NO: 20) | 2.30 | wza | putative polysaccharide export protein, outer |
| | | | membrane |
| STM2181 (SEQ ID NO: 21) | 2.06 | yohJ | putative effector of murein hydrolase LrgA |
| STM2282 (SEQ ID NO: 22) | 2.58 | glpQ | glycerophosphodiester phosphodiesterase, |
| | | | periplasmic |
| STM2314 (SEQ ID NO: 23) | 2.58 | | putative chemotaxis signal transduction protein |
| STM2708 (SEQ ID NO: 24) | 2.03 | | Fels-2 prophage: similar to tail fiber protein |
| | | | (gpI) in |
| | | | phage P2 |
| STM2719 (SEQ ID NO: 25) | 2.12 | | Fels-2 prophage: similar to gpR in phage 186 |
| STM2843 (SEQ ID NO: 26) | 2.03 | hydN | electron transport protein (FeS senter) from |
| | | | formate to |
| | | | hydrogen |
| STM2846 (SEQ ID NO: 27) | 2.26 | hycH | processing of HycE (part of the FHL complex) |
| STM2855 (SEQ ID NO: 28) | 3.25 | hypB | hydrogenase-3 accessory protein, assembly of |
| | | | metallocenter |
| STM4311 (SEQ ID NO: 29) | 3.58 | tnpA | IS200 transposase |
| STM4325 (SEQ ID NO: 30) | 6.39 | dcuA | Dcu family, anaerobic dicarboxylate transport |
| | | | protein |
| STM4415 (SEQ ID NO: 31) | 2.71 | fbp | fructose-bisphosphatase |
| STM4466 (SEQ ID NO: 32) | 4.07 | | putative carbamate kinase |
| SSL_2286 (SEQ ID NO: 33) | 2.97 | orf36 | putative phage replicase |
| Putative, unknown function: | | | |
| STM0289 (SEQ ID NO: 34) | 2.08 | | putative cytoplasmic protein |
| STM0699 (SEQ ID NO: 35) | 2.76 | | putative cytoplasmic protein |
| STM2744 (SEQ ID NO: 36) | 2.44 | | putative cytoplasmic protein |
| STM3752 (SEQ ID NO: 37) | 2.05 | | putative cytoplasmic protein |
| SSL_T1747 (SEQ ID NO: 38) | 4.06 | | putative cytoplasmic protein |
| Plasmid genes: | | | |
| Plasmid 1 (pSLT): | | | |
| PSLT011 (SEQ ID NO: 39) | 2.20 | srgA | sdiA-regulated gene; putative thiol-disulfide |
| | | | isomerase or thioredoxin |
| PSLT015 (SEQ ID NO: 40) | 4.44 | orf5 | putative outer membrane protein |
| PSLT039 (SEQ ID NO: 41) | 2.21 | spvB | Salmonella plasmid virulence: hydrophilic |
| | | | protein |
| PSLT043 (SEQ ID NO: 42) | 4.21 | | putative phosphoribulokinase/uridine kinase |
| | | | family |
| PSLT044 (SEQ ID NO: 43) | 4.43 | | putative integrase protein |
| PSLT054 (SEQ ID NO: 44) | 2.37 | samB | mutagenesis by UV and mutagens; related to |
| | | | umuDC operon |
| PSLT068 (SEQ ID NO: 45) | 2.04 | | putative ParB-like nuclease domain |
| PSLT072 (SEQ ID NO: 46) | 2.11 | | putative transglycosylase |
| PSLT081 (SEQ ID NO: 47) | 4.71 | traB | conjugative transfer: assembly |
| PSLT095 (SEQ ID NO: 48) | 4.24 | traN | conjugative transfer: aggregate stability |
| PSLT099 (SEQ ID NO: 49) | 2.32 | trbB | conjugative transfer |
| PSLT100 (SEQ ID NO: 50) | 2.59 | traH | conjugative transfer: assembly |
| PSLT101 (SEQ ID NO: 51) | 2.20 | traG | conjugative transfer: assembly abd aggregate |
| | | | stability |
| PSLT104 (SEQ ID NO: 52) | 2.87 | traD | conjugative transfer: DNA transport |
| PSLT110 (SEQ ID NO: 53) | 2.37 | traX | conjugative transfer: fimbrial acetylation |
| Plasmid 2: | | | |
| SSL_36 (SEQ ID NO: 54) | 2.02 | colIb | colicin Ib protein |
| SSL_T3 (SEQ ID NO: 55) | 2.68 | trbC | conjugative transfer |
| SSL_T5 (SEQ ID NO: 56) | 3.14 | trbA | conjugative transfer |
| SSL_T12 (SEQ ID NO: 57) | 2.34 | traT | conjugative transfer |
| SSL_T20 (SEQ ID NO: 58) | 2.91 | traK | conjugative transfer |
| SSL_T24 (SEQ ID NO: 59) | 2.10 | traF | conjugative transfer |
| SSL_T35 (SEQ ID NO: 60) | 3.03 | pilL | lipoprotein |
| SSL_T45 (SEQ ID NO: 61) | 2.32 | yagA | unknown function |
| SSL_T52 (SEQ ID NO: 62) | 2.17 | stbA | plasmid stability |
| SSL_T53 (SEQ ID NO: 63) | 5.31 | orf05 | unknown function |
| SSL_T66 (SEQ ID NO: 64) | 2.93 | ygbA | unknown function |
| Plasmid 3: | | | |
| SSL_T69 (SEQ ID NO: 65) | 2.39 | tnpB | putative transposase |
| SSL_T70 (SEQ ID NO: 66) | 14.1 | strB | streptomycin resistance |
| SSL_T71 (SEQ ID NO: 67) | 3.55 | strA | streptomycin resistance |
| SSL_T72 (SEQ ID NO: 68) | 7.43 | sulII | sulphonamide resistance |
| SSL_5085 (SEQ ID NO: 69) | 2.08 | repA | plasmid replication |

DOWN-regulated

| | | | |
|---|---|---|---|
| Protein secretion: | | | |
| STM1153 (SEQ ID NO: 70) | 0.342 | msyB | suppresses protein export mutants |
| STM2895 (SEQ ID NO: 71) | 0.417 | invB | surface presentation of antigens; secretory |
| | | | proteins |
| STM3293 (SEQ ID NO: 72) | 0.432 | secG | preprotein translocase IISP family |
| STM3701 (SEQ ID NO: 73) | 0.473 | secB | molecular chaperone in protein export |
| STM3974 (SEQ ID NO: 74) | 0.445 | tatB | component of Sec-independent protein secretion |
| | | | pathway |
| STM4147 (SEQ ID NO: 75) | 0.392 | secE | preprotein translocase IISP family, membrane |
| | | | subunit |
| Flagella: | | | |
| STM1916 (SEQ ID NO: 76) | 0.458 | cheY | chemotaxis regulator, transmits signals to |
| | | | flagelllar |
| | | | motor |
| STM1925 (SEQ ID NO: 77) | 0.356 | flhD | regulator of flagellar biosynthesis, acts on class 2 |
| | | | operons |
| STM1962 (SEQ ID NO: 78) | 0.443 | fliT | flagellar biosynthesis; possible export chaperone |
| | | | for |
| | | | FliD |
| Fimbrial: | | | |
| STM0543 (SEQ ID NO: 79) | 0.434 | fimA | major type 1 subunit fimbrin (pilin) |
| Stress proteins: | | | |
| STM0831 (SEQ ID NO: 80) | 0.273 | dps | stress response DNA-binding protein |
| STM1652 (SEQ ID NO: 81) | 0.200 | ynaF | putative universal stress protein |
| Regulatory: | | | |
| STM_sRNA (SEQ ID NO: 82) | 0.458 | RFN | putative small regulatory RNA |
| STM_sRNA (SEQ ID NO: 83) | 0.499 | rne5 | putative small regulatory RNA |
| STM_sRNA (SEQ ID NO: 84) | 0.318 | csrB | regulatory RNA |
| STM0473 (SEQ ID NO: 85) | 0.389 | hha | hemolysin expression modulating protein |
| STM0606 (SEQ ID NO: 86) | 0.493 | ybdO | putative transcriptional regulator, LysR family |
| STM0959 (SEQ ID NO: 87) | 0.415 | lrp | regulator for lrp regulon (AsnC family) |
| STM1444 (SEQ ID NO: 88) | 0.391 | slyA | transcriptional regulator for hemolysin (MarR |
| | | | family) |
| STM1660 (SEQ ID NO: 89) | 0.494 | fnr | transcriptional regulator |
| STM2640 (SEQ ID NO: 90) | 0.402 | rpoE | sigma E (sigma 24) factor of RNA polymerase |
| STM3466 (SEQ ID NO: 91) | 0.438 | crp | catabolite activator protein (CAP), cyclic AMP |
| | | | protein (CRP family) |
| STM4315 (SEQ ID NO: 92) | 0.445 | rtsA | AraC-type DNA-binding domain-containing |
| | | | protein |
| STM4361(SEQ ID NO: 93) | 0.298 | hfq | host factor I for bacteriophage Q beta replication |
| Ribosomal: | | | |
| STM0216 (SEQ ID NO: 94) | 0.344 | rpsB | 30S ribosomal subunit protein S2 |
| STM0469 (SEQ ID NO: 95) | 0.474 | rpmE2 | putative 50S ribosomal protein L31 (second |
| | | | copy) |
| STM2675 (SEQ ID NO: 96) | 0.356 | rimM | 16S rRNA processing protein |
| STM3345 (SEQ ID NO: 97) | 0.310 | rplM | 50S ribosomal subunit protein L13 |
| STM3425 (SEQ ID NO: 98) | 0.403 | rplF | 50S ribosomal subunit protein L6 |
| STM3428 (SEQ ID NO: 99) | 0.438 | rplE | 50S ribosomal subunit protein L5 |
| STM3430 (SEQ ID NO: 100) | 0.182 | rplN | 50S ribosomal subunit protein L14 |
| STM3433 (SEQ ID NO: 101) | 0.422 | rplP | 50S ribosomal subunit protein L16 |
| STM3436 (SEQ ID NO: 102) | 0.289 | rpsS | 30S ribosomal subunit protein S19 |
| STM3438 (SEQ ID NO: 103) | 0.457 | rplW | 50S ribosomal subunit protein L23 |
| STM3439 (SEQ ID NO: 104) | 0.393 | rplD | 50S ribosomal subunit protein L4, regulates S10 |
| | | | expression |
| STM3448 (SEQ ID NO: 105) | 0.250 | rpsL | 30S ribosomal subunit protein S12 |
| STM4150 (SEQ ID NO: 106) | 0.423 | rplA | 50S ribosomal subunit protein L1, regulates L1 |
| | | | and |
| | | | L11 |
| STM4391 (SEQ ID NO: 107) | 0.401 | rpsF | 30S ribosomal subunit protein S6 |
| Membrane/periplasmic proteins: | | | |
| STM1164 (SEQ ID NO: 108) | 0.340 | yceB | putative outer membrane lipoprotein |
| STM1249 (SEQ ID NO: 109) | 0.443 | | putative periplasmic protein |
| STM1432 (SEQ ID NO: 110) | 0.464 | ydhO | putative cell wall-associated hydrolase |
| STM1460 (SEQ ID NO: 111) | 0.408 | ydgK | putative inner membrane protein |
| STM1732 (SEQ ID NO: 112) | 0.276 | ompW | outer membrane protein W; colicin S4 receptor |
| STM1798 (SEQ ID NO: 113) | 0.471 | ycgR | putative inner membrane protein |
| STM2505 (SEQ ID NO: 114) | 0.410 | | putative inner membrane protein |
| STM2685 (SEQ ID NO: 115) | 0.411 | smpA | small membrane protein A |
| STM2802 (SEQ ID NO: 116) | 0.453 | ygaM | putative inner membrane protein |
| STM2870 (SEQ ID NO: 117) | 0.462 | | putative inner membrane protein |
| STM3107 (SEQ ID NO: 118) | 0.460 | yggN | putative periplasmic protein |
| STM3228 (SEQ ID NO: 119) | 0.378 | yqjC | putative periplasmic protein |
| STM3229 (SEQ ID NO: 120) | 0.485 | yqjD | putative inner membrane protein |
| STM3231 (SEQ ID NO: 121) | 0.457 | yqjK | putative inner membrane protein |
| STM3347 (SEQ ID NO: 122) | 0.393 | yhcB | putative periplasmic protein |
| STM4378 (SEQ ID NO: 123) | 0.328 | yjfN | putative inner membrane protein |
| STM4561 (SEQ ID NO: 124) | 0.319 | osmY | hyperosmotically inducible periplasmic protein |
| Other function: | | | |
| STM0186 (SEQ ID NO: 125) | 0.406 | dksA | dnaK suppressor protein |
| STM0368 (SEQ ID NO: 126) | 0.386 | prpB | putative carboxyphosphonoenolpyruvate mutase |
| STM0369 (SEQ ID NO: 127) | 0.403 | prpC | putative citrate synthase |
| STM0417 (SEQ ID NO: 128) | 0.382 | ribH | riboflavin synthase, beta chain |
| STM0536 (SEQ ID NO: 129) | 0.483 | ppiB | peptidyl-prolyl cis-trans isomerase B (rotamase |
| | | | B) |
| STM0665 (SEQ ID NO: 130) | 0.480 | gltI | ABC superfamily (bind_prot), |
| | | | glutamate/aspartate |
| | | | transporter |
| STM0759 (SEQ ID NO: 131) | 0.492 | ybgS | putative homeobox protein |
| STM0803 (SEQ ID NO: 132) | 0.408 | moaB | molybdopterin biosynthesis, protein B |
| STM0966 (SEQ ID NO: 133) | 0.497 | dmsC | anaerobic dimethyl sulfoxide reductase, subunit C |
| STM1196 (SEQ ID NO: 134) | 0.336 | acpP | acyl carrier protein |
| STM1291 (SEQ ID NO: 135) | 0.474 | yeaA | putative peptide methionine sulfoxide reductase |
| STM1569 (SEQ ID NO: 136) | 0.458 | fdnH | formate dehydrogenase-N, Fe—S beta subunit, |
| | | | nitrate- |
| | | | inducible |
| STM1783 (SEQ ID NO: 137) | 0.478 | pth | peptidyl-tRNA hydrolase |
| STM2488 (SEQ ID NO: 138) | 0.436 | nlpB | lipoprotein-34 |
| STM2542 (SEQ ID NO: 139) | 0.495 | nifU | NifU homologs involved in Fe—S cluster |
| | | | formation |
| STM2549 (SEQ ID NO: 140) | 0.460 | asrB | anaerobic sulfide reductase |
| STM2646 (SEQ ID NO: 141) | 0.294 | yfiD | putative formate acetyltransferase |
| STM2746 (SEQ ID NO: 142) | 0.344 | | putative Excinuclease ATPase subunit |
| STM2767 (SEQ ID NO: 143) | 0.491 | | putative Superfamily I DNA and RNA helicase |
| STM3039 (SEQ ID NO: 144) | 0.455 | idi | isopentenyldiphosphate isomerase |
| STM3054 (SEQ ID NO: 145) | 0.431 | gcvH | glycine cleavage complex protein H |
| STM3241 (SEQ ID NO: 146) | 0.458 | tdcE | pyruvate formate-lyase 4/2-ketobutyrate |
| | | | formate- |
| | | | lyase |
| STM3443 (SEQ ID NO: 147) | 0.404 | bfr | bacterioferrin, an iron storage homoprotein |
| STM3702 (SEQ ID NO: 148) | 0.453 | grxC | glutaredoxin 3 |
| STM3703 (SEQ ID NO: 149) | 0.321 | yibN | putative Rhodanese-related sulfurtransferases |
| STM3870 (SEQ ID NO: 150) | 0.484 | atpE | membrane-bound ATP synthase, F0 sector, |
| | | | subunit c |
| STM3915 (SEQ ID NO: 151) | 0.492 | trxA | thioredoxin 1, redox factor |
| STM4341 (SEQ ID NO: 152) | 0.411 | frdC | fumarate reductase, anaerobic, membrane anchor |
| | | | polypeptide |
| STM4414 (SEQ ID NO: 153) | 0.475 | ppa | inorganic pyrophosphatase |
| Putative ORF, unknown function: | | | |
| STM0474 (SEQ ID NO: 154) | 0.435 | ybaJ | putative cytoplasmic protein |
| STM1367 (SEQ ID NO: 155) | 0.378 | ydiH | putative cytoplasmic protein |
| STM1583 (SEQ ID NO: 156) | 0.441 | | putative cytoplasmic protein |
| STM2390 (SEQ ID NO: 157) | 0.290 | yfcZ | putative cytoplasmic protein |
| STM2801 (SEQ ID NO: 158) | 0.451 | ygaC | putative cytoplasmic protein |
| STM3461 (SEQ ID NO: 159) | 0.493 | | putative cytoplasmic protein |
| STM3654 (SEQ ID NO: 160) | 0.341 | | pseudogene; in-frame stop following codon 23 |
| STM3995 (SEQ ID NO: 161) | 0.459 | yihD | putative cytoplasmic protein |
| STM4002 (SEQ ID NO: 162) | 0.172 | | putative cytoplasmic protein |
| STM4088 (SEQ ID NO: 163) | 0.357 | yiiU | putative cytoplasmic protein |
| STM4239 (SEQ ID NO: 164) | 0.334 | | putative cytoplasmic protein |
| STM4240 (SEQ ID NO: 165) | 0.411 | yjbJ | putative cytoplasmic protein |
| STM4250 (SEQ ID NO: 166) | 0.422 | yjbQ | putative cytoplasmic protein |
| STM4499 (SEQ ID NO: 167) | 0.494 | yeeN | putative cytoplasmic protein |

TABLE 2
Salmonella typhimurium proteins identified in flight and ground total cell
samples from STS-115 using MudPIT analysis (251 proteins total)

| | | Protein | Ground | Flight |
|---|---|---|---|---|
| | | molecular | total cell | total cell |
| | Accession | weight | protein ID | protein ID |
| Protein name | number | (Daltons) | probability* | probability* |
| aspartate ammonia-lyase | gi\|16767575 (SEQ ID NO: 168) | 52268.1 | 100% | 100% |
| translation elongation factor EF-Tu.A | gi\|96718 (SEQ ID NO: 169) | 43233.6 | 100% | 100% |
| elongation factor G | gi\|16766735 (SEQ ID NO: 170) | 77582 | 100% | 100% |
| putative hydrogenase membrane component precursor | gi\|16764429 (SEQ ID NO: 171) | 37497.2 | 100% | 100% |
| GroEL protein | gi\|16767579 (SEQ ID NO: 172) | 57267.8 | 100% | 100% |
| 30S ribosomal protein S1 | gi\|16764341(SEQ ID NO: 173) | 61154.4 | 100% | 100% |
| L-asparaginase | gi\|16766407 (SEQ ID NO: 174) | 36908.5 | 100% | 100% |
| phosphoenolpyruvate carboxykinase | gi\|16766788 (SEQ ID NO: 175) | 59559.8 | 100% | 100% |
| enolase | gi\|16766258 (SEQ ID NO: 176) | 45468 | 100% | 100% |
| glyceraldehyde 3-phosphate dehydrogenase A | gi\|16764641 (SEQ ID NO: 177) | 35568.6 | 100% | 100% |
| periplasmic glycerophosphodiester phosphodiesterase | gi\|16765609 (SEQ ID NO: 178) | 40407.8 | 100% | 100% |
| molecular chaperone DnaK | gi\|16763402 (SEQ ID NO: 179) | 69241.2 | 100% | 100% |
| 30S ribosomal protein S3 | gi\|16766723 (SEQ ID NO: 180) | 25965.5 | 100% | 100% |
| formate acetyltransferase 1 | gi\|16764333 (SEQ ID NO: 181) | 84989.4 | 100% | 100% |
| 50S ribosomal subunit protein L7/L12 | gi\|16767406 (SEQ ID NO: 182) | 12281 | 100% | 100% |
| ribosomal protein S7 | gi\|16766736 (SEQ ID NO: 183) | 17572.5 | 100% | 100% |
| histone like DNA-binding protein HU-alpha (NS2) (HU-2) | gi\|16767424 (SEQ ID NO: 184) | 9503.1 | 100% | 100% |
| glycerol kinase | gi\|16767352 (SEQ ID NO: 185) | 56046.2 | 100% | 100% |
| dihydrolipoamide dehydrogenase | gi\|16763544 (SEQ ID NO: 186) | 50621.8 | 100% | 100% |
| sn-glycerol-3-phosphate dehydrogenase | gi\|16766813 (SEQ ID NO: 187) | 56908.5 | 100% | 100% |
| trigger factor | gi\|16763828 (SEQ ID NO: 188) | 48048.1 | 100% | 100% |
| cold shock protein | gi\|16765178 (SEQ ID NO: 189) | 7384.3 | 100% | 100% |
| DNA-binding protein HLP-II | gi\|16765095 (SEQ ID NO: 190) | 15525 | 100% | 100% |
| ATP synthase beta subunit | gi\|16767149 (SEQ ID NO: 191) | 50265.5 | 100% | 100% |
| phosphoglycerate kinase | gi\|16766370 (SEQ ID NO: 192) | 41115.1 | 100% | 100% |
| iron-dependent alcohol dehydrogenase AdhE | gi\|16765093 (SEQ ID NO: 193) | 96199.8 | 100% | 100% |
| 50S ribosomal subunit protein L1 | gi\|16767404 (SEQ ID NO: 194) | 24710.5 | 100% | 100% |
| 30S ribosomal protein S4 | gi\|16766705 (SEQ ID NO: 195) | 23467.7 | 100% | 100% |
| 50S ribosomal subunit protein L13 | gi\|16766640 (SEQ ID NO: 196) | 16000.7 | 100% | 100% |
| putative outer membrane porin precursor | gi\|16764916 (SEQ ID NO: 197) | 39662.7 | 99% | 99% |
| FKBP-type peptidyl-prolyl cis-trans isomerase | gi\|16766742 (SEQ ID NO: 198) | 28928.6 | 100% | 100% |
| transketolase 1 isozyme | gi\|16766377 (SEQ ID NO: 199) | 72117 | 100% | 100% |
| 50S ribosomal subunit protein L5 | gi\|16766717 (SEQ ID NO: 200) | 20300.6 | 100% | 100% |
| DNA-directed RNA polymerase beta′ subunit | gi\|16767408 (SEQ ID NO: 201) | 155220 | 100% | 100% |
| 30S ribosomal protein S13 | gi\|16766707 (SEQ ID NO: 202) | 13144.1 | 100% | 100% |
| alkyl hydroperoxide reductase C22 subunit | gi\|16763985 (SEQ ID NO: 203) | 20729.7 | 100% | 100% |
| 30S ribosomal subunit protein S5 | gi\|16766712 (SEQ ID NO: 204) | 17585 | 100% | 100% |
| 50S ribosomal protein L24 | gi\|16766718 (SEQ ID NO: 205) | 11298.3 | 100% | 100% |
| DNA protection during starvation protein | gi\|16764193 (SEQ ID NO: 206) | 18699.8 | 100% | 100% |
| ribosomal protein L19 | gi\|16765988 (SEQ ID NO: 207) | 13112 | 100% | 100% |
| acyl carrier protein | gi\|16764551 (SEQ ID NO: 208) | 8621.4 | 100% | 100% |
| isocitrate dehydrogenase | gi\|16764593 (SEQ ID NO: 209) | 45771 | 100% | 93% |
| triosephosphate isomerase | gi\|16767347 (SEQ ID NO: 210) | 26899 | 100% | 100% |
| 50S ribosomal subunit protein L3 | gi\|16766729 (SEQ ID NO: 211) | 22228.7 | 100% | 100% |
| 30S ribosomal protein S2 | gi\|16763606 (SEQ ID NO: 212) | 26741.2 | 100% | 100% |
| lysine decarboxylase | gi\|16765879 (SEQ ID NO: 213) | 81220.5 | 100% | 100% |
| putative universal stress protein | gi\|16763991 (SEQ ID NO: 214) | 15882.8 | 100% | 100% |
| putative thiol-alkyl hydroperoxide reductase | gi\|16763782 (SEQ ID NO: 215) | 22299 | 100% | 100% |
| 50S ribosomal protein L9 | gi\|16767640 (SEQ ID NO: 216) | 15765.8 | 100% | 100% |
| 50S ribosomal subunit protein L10 | gi\|16767405 (SEQ ID NO: 217) | 17782.8 | 100% | 100% |
| 30S ribosomal subunit protein S16 | gi\|16765991 (SEQ ID NO: 218) | 9216.7 | 100% | 100% |
| 50S ribosomal protein L20 | gi\|16764687 (SEQ ID NO: 219) | 13479.6 | 100% | 100% |
| pyruvate kinase | gi\|16764728 (SEQ ID NO: 220) | 50639.5 | 100% | 98% |
| 6-phosphogluconate dehydrogenase | gi\|16765411 (SEQ ID NO: 221) | 51379.2 | 100% | 93% |
| inorganic pyrophosphatase | gi\|16767660 (SEQ ID NO: 222) | 19658.8 | 100% | 100% |
| 50S ribosomal protein L4 | gi\|16766728 (SEQ ID NO: 223) | 22068.6 | 100% | 100% |
| 50S ribosomal protein L11 | gi\|16767403 (SEQ ID NO: 224) | 14857.5 | 100% | 100% |
| 50S ribosomal subunit protein L17 | gi\|16766703 (SEQ ID NO: 225) | 14377.2 | 100% | 100% |
| succinyl-CoA synthetase beta chain | gi\|16764108 (SEQ ID NO: 226) | 41462.8 | 100% | 100% |
| 50S ribosomal subunit protein L6 | gi\|16766714 (SEQ ID NO: 227) | 18841.3 | 100% | 100% |
| fructose 1,6-bisphosphate aldolase | gi\|16766369 (SEQ ID NO: 228) | 39138.4 | 100% | 100% |
| aconitate hydratase 2 | gi\|16763548 (SEQ ID NO: 229) | 93513.7 | 100% | 100% |
| iron superoxide dismutase | gi\|16764779 (SEQ ID NO: 230) | 21290.4 | 100% | 100% |
| 50S ribosomal protein L22 | gi\|16766724 (SEQ ID NO: 231) | 12208.6 | 100% | 100% |
| sn-glycerol-3-phosphate dehydrogenase large subunit | gi\|16765611 (SEQ ID NO: 232) | 59039.6 | 100% | 100% |
| RNA polymerase, alpha subunit | gi\|16766704 (SEQ ID NO: 233) | 36494.1 | 100% | 100% |
| 30S ribosomal protein S10 | gi\|16766730 (SEQ ID NO: 234) | 11748.8 | 100% | 100% |
| RNA polymerase, beta subunit | gi\|16767407 (SEQ ID NO: 235) | 150586.6 | 100% | 99% |
| polynucleotide phosphorylase | gi\|16766580 (SEQ ID NO: 236) | 77020.9 | 100% | 100% |
| Lpp1 murein lipoprotein | gi\|16764727 (SEQ ID NO: 237) | 8373.6 | 100% | 93% |
| malate dehydrogenase | gi\|16766654 (SEQ ID NO: 238) | 32457.8 | 100% | 100% |
| citrate synthase | gi\|16764100 (SEQ ID NO: 239) | 48089.9 | 100% | 100% |
| GroES protein | gi\|16767578 (SEQ ID NO: 240) | 10300.1 | 100% | 100% |
| putative glutamic dehyrogenase-like protein | gi\|16765136 (SEQ ID NO: 241) | 48020.6 | 100% | 100% |
| succinyl-CoA synthetase alpha subunit | gi\|16764109 (SEQ ID NO: 242) | 29757.8 | 99% | 100% |
| transaldolase B | gi\|16763397 (SEQ ID NO: 243) | 35154.5 | 100% | 100% |
| glycine dehydrogenase | gi\|16766354 (SEQ ID NO: 244) | 104270.1 | 93% | 100% |
| transcription elongation factor NusA | gi\|16766585 (SEQ ID NO: 245) | 55408.3 | 100% | 100% |
| flagellar biosynthesis filament structural protein | gi\|16766083 (SEQ ID NO: 246) | 52518.5 | 100% | 100% |
| elongation factor Ts | gi\|16763607 (SEQ ID NO: 247) | 30339.6 | 100% | 100% |
| N-acetylneuraminate lyase | gi\|16766634 (SEQ ID NO: 248) | 32437.7 | 100% | 100% |
| 50S ribosomal subunit protein L32 | gi\|16764546 (SEQ ID NO: 249) | 6428.4 | 100% | 100% |
| ATP synthase alpha subunit | gi\|16767151 (SEQ ID NO: 250) | 55096 | 100% | 97% |
| 50S ribosomal subunit protein L14 | gi\|16766719 (SEQ ID NO: 251) | 13550.2 | 99% | 100% |
| phosphate acetyltransferase | gi\|16765792 (SEQ ID NO: 252) | 82305.8 | 100% | 100% |
| 50S ribosomal subunit protein L15 | gi\|16766710 (SEQ ID NO: 253) | 14948.9 | 100% | 100% |
| ribose-phosphate pyrophosphokinase | gi\|16765121 (SEQ ID NO: 254) | 34198.6 | 100% | 88% |
| thioredoxin | gi\|16767191** (SEQ ID NO: 255) | 11789.4 | 100% | 0 |
| arginine-binding periplasmic protein 1 precursor | gi\|16764251 (SEQ ID NO: 256) | 26979.4 | 0 | 93% |
| hydrogenase-2 large subunit | gi\|16766447 (SEQ ID NO: 257) | 62420.6 | 100% | 100% |
| cytoplasmic ferritin | gi\|16765276 (SEQ ID NO: 258) | 19262.5 | 100% | 100% |
| riboflavin synthase subunit beta | gi\|16763797 (SEQ ID NO: 259) | 15990.5 | 100% | 97% |
| 50S ribosomal subunit protein L29 | gi\|16766721 (SEQ ID NO: 260) | 7242.6 | 100% | 93% |
| putative universal stress protein | gi\|16764996 (SEQ ID NO: 261) | 15696.7 | 93% | 100% |
| periplasmic nitrate reductase | gi\|16765587 (SEQ ID NO: 262) | 92856.9 | 100% | 93% |
| hyperosmotically-inducible periplasmic protein | gi\|16767802 (SEQ ID NO: 263) | 21430.3 | 93% | 93% |
| ornithine carbamoyltransferase | gi\|16767710 (SEQ ID NO: 264) | 36798.4 | 100% | 100% |
| 30S ribosomal protein S11 | gi\|16766706 (SEQ ID NO: 265) | 13812.8 | 100% | 100% |
| formate dehydrogenase alpha subunit | gi\|16767302 (SEQ ID NO: 266) | 112357.4 | 100% | 100% |
| nucleoside diphosphate kinase (ndk) | gi\|16765846 (SEQ ID NO: 267) | 15503.7 | 100% | 100% |
| putative pyruvate-flavodoxin oxidoreductase | gi\|16764995 (SEQ ID NO: 268) | 128563.7 | 55% | 100% |
| glycoprotein/polysaccharide metabolism protein | gi\|16763846 (SEQ ID NO: 269) | 19458.9 | 100% | 100% |
| O-acetyl serine sulfhydrylase | gi\|11514514 (SEQ ID NO: 270) | 34414.7 | 93% | 100% |
| 30S ribosomal subunit protein S21 | gi\|16766509 (SEQ ID NO: 271) | 8482.1 | 100% | 100% |
| ornithine decarboxylase isozyme | gi\|16764071 (SEQ ID NO: 272) | 82432.6 | 100% | 99% |
| fumarate reductase | gi\|16767591 (SEQ ID NO: 273) | 27157.3 | 100% | 100% |
| anaerobic glycerol-3-phosphate dehydrogenase subunit B | gi\|16765612 (SEQ ID NO: 274) | 45653.3 | 100% | 100% |
| glucose-specific PTS system enzyme IIA component | gi\|16765753 (SEQ ID NO: 275) | 18229.5 | 100% | 100% |
| DNA-directed RNA polymerase omega subunit | gi\|16767026 (SEQ ID NO: 276) | 10218.3 | 100% | 100% |
| FKBP-type peptidyl-prolyl cis-trans isomerase | gi\|16766744 (SEQ ID NO: 277) | 20767.9 | 100% | 100% |
| pyruvate dehydrogenase E1 component | gi\|16763542 (SEQ ID NO: 278) | 99564 | 100% | 97% |
| phosphate acetyltransferase | gi\|16765665 (SEQ ID NO: 279) | 77261.1 | 100% | 93% |
| cold shock-like protein cspE | gi\|16764006 (SEQ ID NO: 280) | 7433.5 | 0 | 93% |
| glucose-6-phosphate isomerase | gi\|16767471 (SEQ ID NO: 281) | 61412.3 | 100% | 93% |
| putative oxidase | gi\|16764715 (SEQ ID NO: 282) | 113118.3 | 93% | 99% |
| 50S ribosomal subunit protein L16 | gi\|16766722 (SEQ ID NO: 283) | 15176.6 | 100% | 100% |
| enterobactin synthetase component F | gi\|16763965 (SEQ ID NO: 284) | 141727.2 | 78% | 99% |
| 50S ribosomal subunit protein L2 | gi\|16766726 (SEQ ID NO: 285) | 29802.1 | 93% | 100% |
| acetyl-coenzyme A carboxylase subunit alpha | gi\|16763622 (SEQ ID NO: 286) | 35327.3 | 100% | 59% |
| aldose 1-epimerase | gi\|16764640 (SEQ ID NO: 287) | 32541.6 | 100% | 0 |
| pyruvate kinase | gi\|16765230 (SEQ ID NO: 288) | 51369.5 | 100% | 93% |
| outer membrane protein C | gi\|16765595** (SEQ ID NO: 289) | 41222.1 | 0 | 93% |
| serine hydroxymethyltransferase | gi\|16765875 (SEQ ID NO: 290) | 45437 | 100% | 93% |
| 50S ribosomal subunit protein L28 | gi\|16767013 (SEQ ID NO: 291) | 9032.6 | 93% | 93% |
| isoaspartyl dipeptidase | gi\|16767756 (SEQ ID NO: 292) | 40306.8 | 100% | 93% |
| sensory histidine kinase | gi\|16765598 (SEQ ID NO: 293) | 106264.3 | 100% | 79% |
| putative inner membrane lipoprotein | gi\|16765852 (SEQ ID NO: 294) | 179631.1 | 87% | 87% |
| Initiation factor IF-3 | gi\|16419853 (SEQ ID NO: 295) | 16619.9 | 100% | 100% |
| putative protease | gi\|16764427 (SEQ ID NO: 296) | 65586.3 | 99% | 97% |
| thiosulfate reductase electron transport protein PhsB | gi\|16765394 (SEQ ID NO: 297) | 21300.7 | 93% | 100% |
| dihydrolipoamide acetyltransferase | gi\|16764107 (SEQ ID NO: 298) | 43839.8 | 100% | 100% |
| protein-export protein SecB | gi\|16766986 (SEQ ID NO: 299) | 17227 | 100% | 100% |
| cytochrome d terminal oxidase polypeptide subunit I | gi\|16764110 (SEQ ID NO: 300) | 58299.5 | 100% | 100% |
| putative detox protein in ethanolamine utilization | gi\|16765785 (SEQ ID NO: 301) | 9824.4 | 100% | 100% |
| galactose transport protein | gi\|16765520 (SEQ ID NO: 302) | 35796.1 | 100% | 99% |
| putative translation initiation inhibitor | gi\|16767703 (SEQ ID NO: 303) | 13557.2 | 100% | 93% |
| dihydrolipoamide acetyltransferase | gi\|16763543 (SEQ ID NO: 304) | 66121.5 | 100% | 93% |
| glutamine ABC transporter periplasmic-binding protein | gi\|16764192 (SEQ ID NO: 305) | 27245.6 | 93% | 93% |
| putative selenocysteine synthase | gi\|16767692 (SEQ ID NO: 306) | 39874.5 | 47% | 93% |
| lysine decarboxylase 2 | gi\|16763624 (SEQ ID NO: 307) | 80747.8 | 0 | 93% |
| putative integral membrane protein | gi\|16764196 (SEQ ID NO: 308) | 59589.3 | 0 | 93% |
| 30S ribosomal protein S20 | gi\|16763433 (SEQ ID NO: 309) | 9637.9 | 100% | 93% |
| NADH dehydrogenase I chain G | gi\|16420864 (SEQ ID NO: 310) | 100254.5 | 100% | 100% |
| acetyl-CoA carboxylase | gi\|16766675 (SEQ ID NO: 311) | 49245.8 | 99% | 100% |
| phosphopentomutase | gi\|16767810 (SEQ ID NO: 312) | 44227.1 | 100% | 0 |
| RNase E | gi\|16764541 (SEQ ID NO: 313) | 119381.7 | 100% | 100% |
| fructose-1,6-bisphosphatase | gi\|16767661 (SEQ ID NO: 314) | 36781.5 | 100% | 100% |
| phosphoenolpyruvate synthase | gi\|16764700 (SEQ ID NO: 315) | 87191.6 | 100% | 100% |
| putative formate acetyltransferase | gi\|16765966 (SEQ ID NO: 316) | 14326.2 | 100% | 93% |
| aminopeptidase B | gi\|16765856 (SEQ ID NO: 317) | 46339 | 0 | 93% |
| lipoprotein | gi\|16765808 (SEQ ID NO: 318) | 36919.8 | 100% | 93% |
| D-ribose-binding protein | gi\|1070661 (SEQ ID NO: 319) | 28512.8 | 93% | 0 |
| oriT nickase/helicase | gi\|16445291 (SEQ ID NO: 320) | 191664 | 100% | 0 |
| sensory transduction histidine kinase | gi\|16764736 (SEQ ID NO: 321) | 65221.8 | 93% | 54% |
| sensory kinase in two-component system with CreB | gi\|16767830 (SEQ ID NO: 322) | 51666.7 | 93% | 59% |
| agmatinase | gi\|16766379 (SEQ ID NO: 323) | 33585.5 | 100% | 100% |
| aminoacyl-histidine dipeptidase | gi\|16763698 (SEQ ID NO: 324) | 52419.9 | 93% | 100% |
| DNA polymerase I | gi\|16767264 (SEQ ID NO: 325) | 103114.9 | 93% | 0 |
| glycerate kinase II | gi\|16763905 (SEQ ID NO: 326) | 39003.1 | 0 | 99% |
| 30S ribosomal subunit protein S19 | gi\|16766725 (SEQ ID NO: 327) | 10398.5 | 100% | 93% |
| ATPase subunit | gi\|7594817 (SEQ ID NO: 328) | 46171.1 | 100% | 0 |
| glucose-1-phosphate adenylyltransferase | gi\|16766822 (SEQ ID NO: 329) | 48444.5 | 100% | 83% |
| phosphoglyceromutase | gi\|16766989 (SEQ ID NO: 330) | 56237.5 | 100% | 0 |
| acetate kinase | gi\|16765664 (SEQ ID NO: 331) | 43240.3 | 100% | 92% |
| ABC superfamily peptide transport protein | gi\|16765039 (SEQ ID NO: 332) | 30654.8 | 48% | 100% |
| potassium-transporting ATPase subunit B | gi\|16764075 (SEQ ID NO: 333) | 72125 | 99% | 74% |
| adenylosuccinate synthetase | gi\|16767612 (SEQ ID NO: 334) | 47359.7 | 100% | 100% |
| putative periplasmic protein | gi\|16765796 (SEQ ID NO: 335) | 38706.3 | 100% | 0 |
| ATP synthase delta subunit | gi\|16767152 (SEQ ID NO: 336) | 19394.2 | 97% | 100% |
| glycerol dehydrogenase | gi\|16767374 (SEQ ID NO: 337) | 38723.5 | 100% | 96% |
| inositol-5-monophosphate dehydrogenase | gi\|16765831 (SEQ ID NO: 338) | 51930.1 | 100% | 0 |
| succinate dehydrogenase catalytic subunit | gi\|16764105 (SEQ ID NO: 339) | 26847.2 | 100% | 93% |
| FkbP-type peptidyl-prolyl cis-trans isomerase | gi\|16767643 (SEQ ID NO: 340) | 23719.2 | 100% | 93% |
| putative sigma(54) modulation protein | gi\|16765980 (SEQ ID NO: 341) | 12634.6 | 100% | 93% |
| putative ATP-dependent helicase | gi\|16765162 (SEQ ID NO: 342) | 70268.1 | 93% | 0 |
| ATP synthase epsilon subunit | gi\|16767148 (SEQ ID NO: 343) | 15046.6 | 100% | 93% |
| putative cytoplasmic protein | gi\|16765717 (SEQ ID NO: 344) | 10269.4 | 100% | 93% |
| needle complex major subunit PrgI | gi\|16766179 (SEQ ID NO: 345) | 8839.3 | 93% | 100% |
| cytochrome o ubiquinol oxidase subunit I | gi\|16763823 (SEQ ID NO: 346) | 74265.7 | 93% | 100% |
| hydrogenase 3 large subunit | gi\|16766155 (SEQ ID NO: 347) | 65003.1 | 100% | 0 |
| mannose-specific enzyme IIAB | gi\|16765171 (SEQ ID NO: 348) | 34969.2 | 100% | 0 |
| 30s ribosomal protein S6 | gi\|16767637** (SEQ ID NO: 349) | 15154.9 | 100% | 0 |
| fumarate reductase, flavoprotein subunit | gi\|16767592 (SEQ ID NO: 350) | 65473.9 | 93% | 100% |
| putative lipoprotein | gi\|16764058 (SEQ ID NO: 351) | 12218.6 | 72% | 96% |
| dipeptide transport protein | gi\|16766917 (SEQ ID NO: 352) | 60202.5 | 100% | 0 |
| phase 1 flagellin | gi\|16765297 (SEQ ID NO: 353) | 51594.5 | 100% | 100% |
| transcription termination factor Rho | gi\|16767192 (SEQ ID NO: 354) | 46977.2 | 100% | 100% |
| arginine deiminase | gi\|16767712 (SEQ ID NO: 355) | 45544.5 | 99% | 100% |
| D-fructose-6-phosphate amidotransferase | gi\|16767145 (SEQ ID NO: 356) | 66860.7 | 100% | 0 |
| tetrathionate reductase subunit A (TtrA) | gi\|16764733 (SEQ ID NO: 357) | 110976.9 | 97% | 0 |
| uridine phosphorylase | gi\|16422527 (SEQ ID NO: 358) | 27205.9 | 100% | 0 |
| ecotin precursor | gi\|16765590 (SEQ ID NO: 359) | 18199.7 | 0 | 100% |
| anaerobic dimethyl sulfoxide reductase chain B | gi\|16764326** (SEQ ID NO: 360) | 22761.6 | 100% | 0 |
| serine endoprotease | gi\|16766643 (SEQ ID NO: 361) | 47310.1 | 93% | 100% |
| putative fructose-1,6-bisphosphate aldolase | gi\|16767344 (SEQ ID NO: 362) | 31725.2 | 93% | 100% |
| NADH dehydrogenase I chain F | gi\|16765651 (SEQ ID NO: 363) | 49229.2 | 100% | 93% |
| small membrane protein A | gi\|16766000 (SEQ ID NO: 364) | 12327 | 93% | 100% |
| single-strand DNA-binding protein | gi\|16767506 (SEQ ID NO: 365) | 19055.5 | 93% | 100% |
| aldehyde oxidoreductase | gi\|3885918 (SEQ ID NO: 366) | 49239.5 | 93% | 100% |
| virulence-associated protein mkfB | gi\|7443056 (SEQ ID NO: 367) | 62570.6 | 100% | 0 |
| 3,4-dihydroxy-2-butanone 4-phosphate synthase | gi\|16766495 (SEQ ID NO: 368) | 23292.3 | 0 | 86% |
| PilQ ATP-binding protein | gi\|32470257 (SEQ ID NO: 369) | 58267.7 | 98% | 86% |
| prolyl-tRNA synthetase | gi\|16763631 (SEQ ID NO: 370) | 63522.6 | 100% | 0 |
| BipA GTPase | gi\|16767274 (SEQ ID NO: 371) | 67359.4 | 100% | 66% |
| 2-oxoglutarate dehydrogenase | gi\|16764106 (SEQ ID NO: 372) | 104805.9 | 0 | 100% |
| cytosine deaminase | gi\|16766629 (SEQ ID NO: 373) | 47608 | 100% | 0 |
| ribosome recycling factor | gi\|16763609 (SEQ ID NO: 374) | 20538 | 100% | 0 |
| dihydrodipicolinate synthase | gi\|16765809** (SEQ ID NO: 375) | 31276.4 | 100% | 0 |
| putative dehydrogenase | gi\|16765715 (SEQ ID NO: 376) | 77238.8 | 99% | 0 |
| TrpR binding protein WrbA | gi\|16764477 (SEQ ID NO: 377) | 20849.7 | 100% | 93% |
| outer membrane-bound fatty acid transporter | gi\|16765718 (SEQ ID NO: 378) | 47688.5 | 93% | 0 |
| catalase HPII | gi\|16764669 (SEQ ID NO: 379) | 83610.2 | 93% | 100% |
| putative cytoplasmic protein | gi\|16763672** (SEQ ID NO: 380) | 79560.5 | 99% | 0 |
| D-ribose-binding periplasmic protein | gi\|16767168 (SEQ ID NO: 381) | 30944.8 | 0 | 99% |
| 2-dehydro-3-deoxyphosphooctonate aldolase | gi\|16765113 (SEQ ID NO: 382) | 30777.4 | 93% | 0 |
| flagellar hook-associated protein | gi\|16765298 (SEQ ID NO: 383) | 49818.7 | 0 | 87% |
| cysteine desulfurase | gi\|16765863 (SEQ ID NO: 384) | 45075.7 | 0 | 83% |
| ethanolamine utilization protein EutL | gi\|16765776 (SEQ ID NO: 385) | 22678 | 100% | 81% |
| nikB plasmid protein | gi\|20521580 (SEQ ID NO: 386) | 103992.3 | 79% | 100% |
| periplasmic maltose-binding protein | gi\|16767479 (SEQ ID NO: 387) | 43468 | 100% | 0 |
| hydrogenase-3 accessory protein | gi\|16766161** (SEQ ID NO: 388) | 31375.2 | 0 | 99% |
| chemotactic response protein | gi\|16765257** (SEQ ID NO: 389) | 23902.4 | 100% | 0 |
| putative acetyltransferase | gi\|16765805 (SEQ ID NO: 390) | 74000 | 100% | 0 |
| putative imidazolonepropionase or amidohydrolase | gi\|16767659 (SEQ ID NO: 391) | 42408 | 0 | 100% |
| phosphoglucosamine mutase | gi\|16766590 (SEQ ID NO: 392) | 47424.2 | 100% | 0 |
| ATP-dependent RNA helicase | gi\|16765963 (SEQ ID NO: 393) | 50040.2 | 100% | 0 |
| asparagine synthetase B | gi\|16764050 (SEQ ID NO: 394) | 62555.9 | 0 | 100% |
| 50S ribosomal subunit protein L30 | gi\|16766711** (SEQ ID NO: 395) | 6495.8 | 0 | 100% |
| glutamine synthetase | gi\|16767272 (SEQ ID NO: 396) | 51768.7 | 100% | 0 |
| outer membrane protein Tsx | gi\|16763793 (SEQ ID NO: 397) | 32761.5 | 100% | 0 |
| ribonuclease R (RNase R) | gi\|16767614 (SEQ ID NO: 398) | 92033.1 | 99% | 0 |
| DNA-binding protein HU-beta | gi\|16763832 (SEQ ID NO: 399) | 9222 | 99% | 0 |
| 50S ribosomal subunit protein L23 | gi\|16766727** (SEQ ID NO: 400) | 11194.9 | 0 | 100% |
| membrane-bound ATP synthase, epsilon-subunit | gi\|6625704 (SEQ ID NO: 401) | 14848.1 | 0 | 100% |
| hydrogenase-2 small chain protein | gi\|16766450 (SEQ ID NO: 402) | 39604.2 | 0 | 100% |
| ATP synthase subunit C | gi\|16767150 (SEQ ID NO: 403) | 31538 | 0 | 100% |
| putative zinc-binding dehydrogenase | gi\|16764887 (SEQ ID NO: 404) | 37229 | 100% | 0 |
| transcriptional repressor for rbs operon (GalR/LacI family) | gi\|16767170 (SEQ ID NO: 405) | 36702.1 | 100% | 0 |
| ubiquinone/menaquinone methyltransferase UbiE | gi\|16767240 (SEQ ID NO: 406) | 28118.9 | 100% | 0 |
| phosphoheptose isomerase | gi\|16763693 (SEQ ID NO: 407) | 20878.5 | 100% | 0 |
| ClpB ATP-dependent protease | gi\|16765976 (SEQ ID NO: 408) | 95421.8 | 99% | 0 |
| putative pyrophosphatase | gi\|16766260 (SEQ ID NO: 409) | 30812.9 | 99% | 0 |
| precorrin-8X methylmutase | gi\|16765363 (SEQ ID NO: 410) | 23016.8 | 0 | 99% |
| translation initiation factor IF-2 | gi\|16766584 (SEQ ID NO: 411) | 97383.5 | 0 | 99% |
| putative GTP-binding protein | gi\|16766597 (SEQ ID NO: 412) | 43086.7 | 99% | 0 |
| ethanolamine ammonia-lyase heavy chain | gi\|16765778 (SEQ ID NO: 413) | 49432 | 0 | 99% |
| putative copper-transporting ATPase | gi\|16763878 (SEQ ID NO: 414) | 87893 | 0 | 99% |
| putative 5′-nucleotidase/2′,3′-cyclic phosphodiesterase | gi\|16767370 (SEQ ID NO: 415) | 56560 | 98% | 0 |
| hypothetical ABC transporter ATP-binding protein | gi\|16763887 (SEQ ID NO: 416) | 24467.3 | 98% | 0 |
| putative glycosyl transferase | gi\|16765625 (SEQ ID NO: 417) | 36500 | 98% | 0 |
| citrate lyase alpha chain | gi\|56967225 (SEQ ID NO: 418) | 54561.7 | 97% | 0 |

*Peptide samples obtained from MudPIT were analyzed using Sequest and X!Tandem software, and the data organized using the Scaffold program. To be considered a positive identification in Scaffold, the following parmeters were used: a minumum of 2 peptides from a given protein identified with peptide and protein thresholds of 80% to give an overall protein identification (ID) probability of at least 80%. Note that a protein ID probability of greater than 80% in at least one of the samples warranted inclusion in the table so as to allow identification of possible differential expression of a given protein.
**Proteins identified via MuDPIT analysis as differentially expressed that also displayed differential expression via microarray analysis.

TABLE 3
Spaceflight stimulon genes belonging to Hfq regulon or involved with iron utilization or biofilm formation

| | Fold | |
|---|---|---|
| Gene* | change | Function |
| Hfq regulon genes | | |
| Up-regulated | | |
| Outer membrane proteins | | |
| ompA (SEQ ID NO: 419) | 2.05 | outer membrane porin |
| ompC (SEQ ID NO: 420) | 2.44 | outer membrane porin |
| ompD (SEQ ID NO: 421) | 3.34 | outer membrane porin |
| Plasmid transfer apparatus | | |
| traB (SEQ ID NO: 422) | 4.71 | conjugative transfer, assembly |
| traN (SEQ ID NO: 423) | 4.24 | conjugative transfer, aggregate formation |
| trbA (SEQ ID NO: 424) | 3.14 | conjugative transfer |
| traK (SEQ ID NO: 425) | 2.91 | conjugative transfer |
| traD (SEQ ID NO: 426) | 2.87 | conjugative transfer, DNA transport |
| trbC (SEQ ID NO: 427) | 2.68 | conjugative transfer |
| traH (SEQ ID NO: 428) | 2.59 | conjugative transfer, assembly |
| traX (SEQ ID NO: 429) | 2.37 | conjugative transfer, fimbrial acetylation |
| traT (SEQ ID NO: 430) | 2.34 | conjugative transfer |
| trbB (SEQ ID NO: 431) | 2.32 | conjugative transfer |
| traG (SEQ ID NO: 432) | 2.21 | conjugative transfer, assembly |
| traF (SEQ ID NO: 433) | 2.11 | conjugative transfer |
| traR (SEQ ID NO: 434) | 1.79 | conjugative transfer |
| Various cellular functions | | |
| gapA (SEQ ID NO: 435) | 7.67 | glyceraldehyde-3-phosphate dehydrogenase A |
| sipC (SEQ ID NO: 436) | 6.27 | cell invasion protein |
| adhE (SEQ ID NO: 18) | 4.75 | iron-dependent alcohol dehydrogenase of AdhE |
| glpQ (SEQ ID NO: 22) | 2.58 | glycerophosphodiester phosphodiesterase, periplasmic |
| fliC (SEQ ID NO: 1) | 2.11 | flagellin, filament structural protein |
| sbmA (SEQ ID NO: 437) | 1.67 | putative ABC superfamily transporter |
| Down-regulated | | |
| Small RNAs | | |
| alpha RBS (SEQ ID NO: 438) | 0.305 | small RNA |
| rnaseP (SEQ ID NO: 439) | 0.306 | small RNA regulatory |
| csrB (SEQ ID NO: 84) | 0.318 | small RNA regulatory |
| tke1 (SEQ ID NO: 440) | 0.427 | small RNA |
| oxyS (SEQ ID NO: 441) | 0.432 | small RNA regulatory |
| RFN (SEQ ID NO: 442) | 0.458 | small RNA |
| rne5 (SEQ ID NO: 443) | 0.499 | small RNA |
| Ribosomal proteins | | |
| rpsL (SEQ ID NO: 105) | 0.251 | 30S ribosomal subunit protein S12 |
| rpsS (SEQ ID NO: 102) | 0.289 | 30S ribosomal subunit protein S19 |
| rplD (SEQ ID NO: 104) | 0.393 | 50S ribosomal subunit protein L4 |
| rpsF (SEQ ID NO: 107) | 0.401 | 30S ribosomal subunit protein S6 |
| rplP (SEQ ID NO: 101) | 0.422 | 50S ribosomal subunit protein L16 |
| rplA (SEQ ID NO: 106) | 0.423 | 50S ribosomal subunit protein L1 |
| rpme2 (SEQ ID NO: 95) | 0.473 | 50S ribosomal protein L31 (second copy) |
| rplY (SEQ ID NO: 444) | 0.551 | 50S ribosomal subunit protein L25 |
| Various cellular functions | | |
| ynaF (SEQ ID NO: 81) | 0.201 | putative universal stress protein |
| ygfE (SEQ ID NO: 445) | 0.248 | putative cytoplasmic protein |
| dps (SEQ ID NO: 80) | 0.273 | stress response DNA-binding protein |
| hfq (SEQ ID NO: 446) | 0.298 | host factor for phage replication, RNA chaperone |
| osmY (SEQ ID NO: 124) | 0.318 | hyperosmotically inducible periplasmic protein |
| mysB (SEQ ID NO: 70) | 0.341 | suppresses protein export mutants |
| rpoE (SEQ ID NO: 90) | 0.403 | sigma E (sigma 24) factor of RNA polymerase |
| cspD (SEQ ID NO: 447) | 0.421 | similar to CspA but not cold shock induced |
| Nlpb (SEQ ID NO: 138) | 0.435 | lipoprotein-34 |
| ygaC (SEQ ID NO: 158) | 0.451 | putative cytoplasmic protein |
| ygaM (SEQ ID NO: 116) | 0.453 | putative inner membrane protein |
| gltI (SEQ ID NO: 130) | 0.479 | ABC superfamily, glutamate/aspartate transporter |
| ppiB (SEQ ID NO: 129) | 0.482 | peptidyl-prolyl cis-trans isomerase B (rotamase B) |
| atpE (SEQ ID NO: 150) | 0.482 | membrane-bound ATP synthase, F0 sector, subunit c |
| yfiA (SEQ ID NO: 448) | 0.482 | ribosome associated factor, stabilizes against dissociation |
| trxA (SEQ ID NO: 151) | 0.493 | thioredoxin 1, redox factor |
| nifU (SEQ ID NO: 139) | 0.496 | NifU homologs involved in Fe—S cluster formation |
| rbfA (SEQ ID NO: 449) | 0.506 | ribosome-binding factor, role in processing of 10S rRNA |
| rseB (SEQ ID NO: 450) | 0.514 | anti-sigma E factor |
| yiaG (SEQ ID NO: 451) | 0.528 | putative transcriptional regulator |
| ompX (SEQ ID NO: 452) | 0.547 | outer membrane protease, receptor for phage OX2 |
| rnpA (SEQ ID NO: 453) | 0.554 | RNase P, protein component (protein C5) |
| hns (SEQ ID NO: 454) | 0.554 | DNA-binding protein; pleiotropic regulator |
| lamB (SEQ ID NO: 455) | 0.566 | phage lambda receptor protein; maltose high-affinity receptor |
| rmf (SEQ ID NO: 456) | 0.566 | ribosome modulation factor |
| tpx (SEQ ID NO: 457) | 0.566 | thiol peroxidase |
| priB (SEQ ID NO: 458) | 0.571 | primosomal replication protein N |
| Iron utilization/storage genes | | |
| adhE (SEQ ID NO: 18) | 4.76 | iron-dependent alcohol dehydrogenase of AdhE |
| entE (SEQ ID NO: 14) | 2.24 | 2,3-dihydroxybenzoate-AMP ligase |
| hydN (SEQ ID NO: 26) | 2.03 | electron transport protein (FeS senter) from formate to hydrogen |
| dmsC (SEQ ID NO: 133) | 0.497 | anaerobic dimethyl sulfoxide reductase, subunit C |
| nifU (SEQ ID NO: 139) | 0.495 | NifU homologs involved in Fe—S cluster formation |
| Fnr (SEQ ID NO: 89) | 0.494 | transcriptional regulator, iron-binding |
| fdnH (SEQ ID NO: 136) | 0.458 | formate dehydrogenase-N, Fe—S beta subunit, nitrate-inducible |
| frdC (SEQ ID NO: 152) | 0.411 | fumarate reductase, anaerobic, membrane anchor polypeptide |
| Bfr (SEQ ID NO: 147) | 0.404 | bacterioferrin, an iron storage homoprotein |
| ompW (SEQ ID NO: 112) | 0.276 | outer membrane protein W; colicin S4 receptor |
| Dps (SEQ ID NO: 80) | 0.273 | stress response DNA-binding protein and ferritin |
| Genes implicated in/associated | | |
| with biofilm formation | | |
| Wza (SEQ ID NO: 20) | 2.30 | putative polysaccharide export protein, outer membrane |
| wcaI (SEQ ID NO: 19) | 2.07 | putative glycosyl transferase in colanic acid biosynthesis |
| ompA (SEQ ID NO: 6) | 2.06 | outer membrane protein |
| wcaD (SEQ ID NO: 459) | 1.82 | putative colanic acid polymerase |
| wcaH (SEQ ID NO: 460) | 1.76 | GDP-mannose mannosyl hydrolase in colanic acid biosynthesis |
| manC (SEQ ID NO: 461) | 1.71 | mannose-1-phosphate guanylyltransferase |
| wcaG (SEQ ID NO: 462) | 1.68 | bifunctional GDP fucose synthetase in colanic acid biosyntheis |
| wcaB (SEQ ID NO: 463) | 1.64 | putative acyl transferase in colanic acid biosynthesis |
| fimH (SEQ ID NO: 464) | 1.61 | fimbrial subunit |
| fliS (SEQ ID NO: 465) | 0.339 | flagellar biosynthesis |
| flgM (SEQ ID NO: 466) | 0.343 | flagellar biosynthesis |
| flhD (SEQ ID NO: 467) | 0.356 | flagellar biosynthesis |
| fliE (SEQ ID NO: 468) | 0.438 | flagellar biosynthesis |
| fliT (SEQ ID NO: 469) | 0.444 | flagellar biosynthesis |
| cheY (SEQ ID NO: 76) | 0.461 | chemotaxic response |
| cheZ (SEQ ID NO: 470) | 0.535 | chemotaxic response |

### REFERENCES

1. Grigoriev, A. I., Svetaylo, E. N. & Egorov, A. D. (1998) Environ Med 42, 83-94.
2. Sonnenfeld, G. & Shearer, W. T. (2002) Nutrition 18, 899-903.
3. Taylor, G. R. (1993) J Leukoc Biol 54, 202-8.
4. Taylor, G. R., Konstantinova, I., Sonnenfeld, G. & Jennings, R. (1997) Adv Space Biol Med 6, 1-32.
5. Nickerson, C. A., Ott, C. M., Mister, S. J., Morrow, B. J., Burns-Keliher, L. & Pierson, D. L. (2000) Infect Immun 68, 3147-52.
6. Nickerson, C. A., Ott, C. M., Wilson, J. W., Ramamurthy, R. & Pierson, D. L. (2004) Microbiol Mol Biol Rev 68, 345-61.
7. Wilson, J. W., Ramamurthy, R., Porwollik, S., McClelland, M., Hammond, T., Allen, P., Ott, C. M., Pierson, D. L. & Nickerson, C. A. (2002) Proc Natl Acad Sci USA 99, 13807-12.
8. Figueroa-Bossi, N., Lemire, S., Maloriol, D., Balbontin, R., Casadesus, J. & Bossi, L. (2006) Mol Microbiol 62, 838-52.
9. Masse, E. & Gottesman, S. (2002) Proc Natl Acad Sci USA 99, 4620-5.
10. Muffler, A., Traulsen, D. D., Fischer, D., Lange, R. & Hengge-Aronis, R. (1997) J Bacteriol 179, 297-300.
11. Vecerek, B., Moll, I., Afonyushkin, T., Kaberdin, V. & Blasi, U. (2003) Mol Microbiol 50, 897-909.
12. Guisbert, E., Rhodius, V. A., Ahuja, N., Witkin, E. & Gross, C. A. (2007) J Bacteriol 189, 1963-73.
13. Sittka, A., Pfeiffer, V., Tedin, K. & Vogel, J. (2007) Mol Microbiol 63, 193-217.
14. Valentin-Hansen, P., Eriksen, M. & Udesen, C. (2004) Mol Microbiol 51, 1525-33.
15. Valentin-Hansen, P., Johansen, J. & Rasmussen, A. A. (2007) Curr Opin Microbiol 10, 152-5.
16. Will, W. R. & Frost, L. S. (2006) J Bacteriol 188, 124-31.
17. Hammond, T. G. & Hammond, J. M. (2001) Am J Physiol Renal Physiol 281, F12-25.
18. Nickerson, C. A. & Ott, C. M. (2004) ASM News 70, 169-175.
19. Nickerson, C. A., Ott, C. M., Wilson, J. W., Ramamurthy, R., LeBlanc, C. L., Honer zu Bentrup, K., Hammond, T. & Pierson, D. L. (2003) J Microbiol Methods 54, 1-11.
20. Little, B., Wagner, P., Ray, R., Pope, R. & Scheetz, R. (1991) J Ind Microbiol 8, 213-222.
21. Priester, J. H., Horst, A. M., Van de Werfhorst, L. C., Saleta, J. L., Mertes, L. A. & Holden, P. A. (2007) J Microbiol Methods 68, 577-87.
22. Wilson, J. W. & Nickerson, C. A. (2006) BMC Evol Biol 6, 2.
23. Reed, L. J. & Muench, H. (1938) Am J Hyg 27, 493-497.
24. Nickerson, C. A., Goodwin, T. J., Terlonge, J., Ott, C. M., Buchanan, K. L., Uicker, W. C., Emami, K., LeBlanc, C. L., Ramamurthy, R., Clarke, M. S., Vanderburg, C. R., Hammond, T. & Pierson, D. L. (2001) Infect Immun 69, 7106-20.
25. Navarre, W. W., Porwollik, S., Wang, Y., McClelland, M., Rosen, H., Libby, S. J. & Fang, F. C. (2006) Science 313, 236-8.
26. Zhang, A., Wassarman, K. M., Rosenow, C., Tjaden, B. C., Storz, G. & Gottesman, S. (2003) Mol Microbiol 50, 1111-24.
27. Cooper, B., Eckert, D., Andon, N. L., Yates, J. R. & Haynes, P. A. (2003) J Am Soc Mass Spectrom 14, 736-41.
28. Qian, W. J., Liu, T., Monroe, M. E., Strittmatter, E. F., Jacobs, J. M., Kangas, L. J., Petritis, K., Camp, D. G., 2nd & Smith, R. D. (2005) J Proteome Res 4, 53-62.
29. Craig, R. & Beavis, R. C. (2004) Bioinformatics 20, 1466-7.
30. Eng, J. K., McCormack, A. L. & Yates, J. R. (1994) J Am Soc Mass Spectrom 5, 976-989.

### Example 2

### Media Ion Content Inhibits Increased Microbial Virulence During Spaceflight

This example describes experiments designed to test the hypothesis that ion concentrations could be manipulated to prevent the enhanced Salmonella virulence imparted during flight. Salmonella cultured in varying media conditions aboard STS-115 and STS-123 were analyzed. These experiments allowed the identification of a) media ion composition that prevents spaceflight-induced increases in Salmonella virulence, and b) commonalities and differences in Salmonella gene expression between growths of the same pathogen in different media during spaceflight. As with spaceflight growth in LB, Salmonella grown in M9 media during flight displayed differential expression of many genes, including those associated with either the regulation of, or regulation by the Hfq protein and small regulatory RNAs. Salmonella grown in various media demonstrated that ion concentrations had a direct effect on the virulence of the cultures. Moreover, higher concentrations of phosphate ions present in M9 medium during spaceflight analogue culture altered its pathogenic-related effects, thus providing the first evidence of a mechanism behind this response.

### Material and Methods

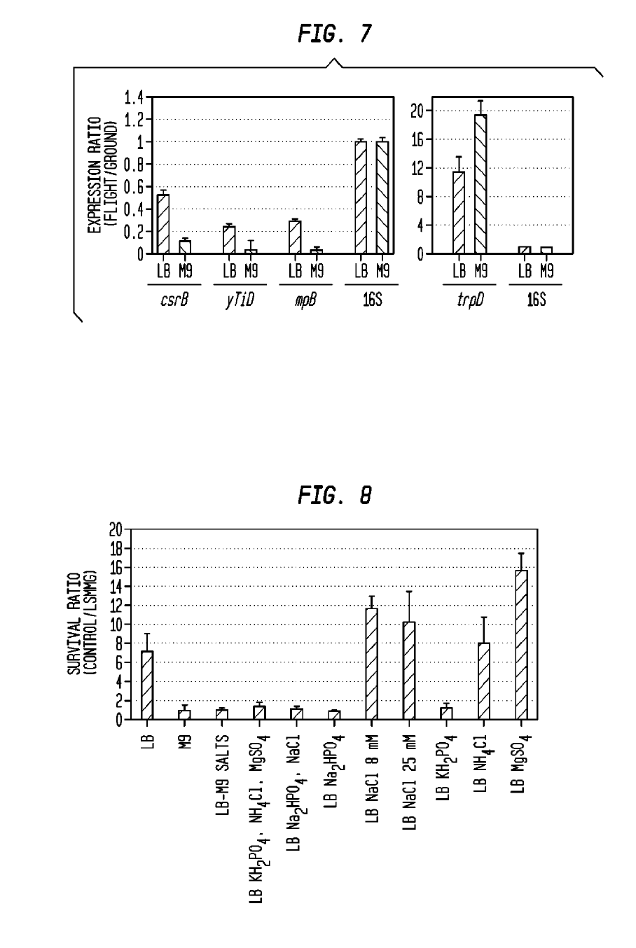

Strains and Media. The virulent, mouse-passaged Salmonella typhimurium derivative of SL1344 termed F3339 was used in all experiments¹⁸. Lennox broth (LB) (10 g tryptone, 5 g yeast extract, 5 g NaCl)¹⁹, M9 medium (0.4% glucose)⁹, or LB-M9 salts medium were used as the growth media in all experiments. Phosphate buffered saline (PBS) (Invitrogen, Carlsbad, Calif.) was used to resuspend bacteria for use as inoculum in the flight and ground hardware. The LB-M9 salts medium consisted of LB medium supplemented with the following amounts of ions: 8.54 mM NaCl, 25.18 mM NaH₂PO₄, 18.68 mM NH₄Cl, 22 mM KH₂PO₄, and 2 mM MgSO₄. The RNA fixative RNA Later II (Ambion, Austin, Tex.), was used to preserve nucleic acid and protein. Bacterial cell culture, microarray analysis, MudPIT proteomics, murine infections, and acid stress assays were performed as described previously¹. qRT-PCR analysis was performed with primers hybridizing to the indicated genes as described previously using the 16S rRNA gene to normalize samples²⁰. Data from three to nine separate technical replicate reactions was used for each gene in FIG. 7, and the differences in expression were found to be statistically significant using student's t-test (p-value<0.05). The sequences of the primers used here are as follows. Determination of inorganic ion levels in LB and M9 media was performed using inductively coupled plasma (ICP) spectrometry and ion chromatography (IC) as described previously²¹.

Primers used in this study
for qRT-PCR

5Sal16S
(SEQ ID No: 865)
gtaacggctcaccaaggcgacgatccctag
Sal16S3
(SEQ ID No: 866)
cttcgccaccggtattcctccagatctctac
5STM1724 (for trpD)
(SEQ ID No: 867)
agcgcctttgtcgcggcggcctgtgga
STM17243 (for trpD)
(SEQ ID No: 868)
gttgatcagcgggccgagtacgttgaacag
5rnpB
(SEQ ID No: 869)
gtcgtggacagtcattcatctaggccagca
rnpB3
(SEQ ID No: 870)
ctccatagggcagggtgccaggtaacgcct
5csrB
(SEQ ID No: 871)
tttcctgtgaccttacggcctgttcatcctg
csrB3
(SEQ ID No: 872)
agcaggacacgccaggatggtgttacaagg
5yfiD
(SEQ ID No: 873)
tacgagcgataacgtcgcgctgctgttccg
yfiD3
(SEQ ID No: 874)
gctgaattccttctggctgctggacagcga

Bacterial cell culture. Spaceflight and ground cultures were grown in specialized hardware termed fluid processing apparatus (FPA) as described previously¹. Briefly, an FPA consists of a glass barrel that can be divided into compartments via the insertion of rubber stoppers and a lexan sheath into which the glass barrel is inserted. Each compartment in the glass barrel was filled with a solution in an order such that the solutions would be mixed at specific time points in flight via two actions: (1) downward plunging action on the rubber stoppers and (2) passage of the fluid in a given compartment through a bevel on the side of the glass barrel such that it was released into the compartment below. Glass barrels and rubber stoppers were coated with a silicone lubricant (Sigmacote, Sigma, St. Louis, Mo.) and autoclaved separately before assembly. A stopper with a gas exchange membrane was inserted just below the bevel in the glass barrel before autoclaving. FPA assembly was performed aseptically in a laminar flow hood in the following order: 2.0 ml media (either LB, M9 or LB-M9) on top of the gas exchange stopper, one rubber stopper, 0.5 ml PBS containing bacterial inoculum (approximately 6.7×10⁶ bacteria), another rubber stopper, 2.5 ml of either RNA fixative (for gene expression analysis) or media (either LB, M9 or LB-M9 for virulence studies), and a final rubber stopper. Syringe needles (gauge 25⅝) were inserted into rubber stoppers during this process to release air pressure and facilitate assembly. To facilitate group activation of FPAs and to ensure proper containment levels, sets of 8 FPAs were loaded into larger containers termed group activation packs (GAPs). All ground control cultures were incubated in the Orbital Environmental Simulator (OES) room at the Kennedy Space Center, which is linked in real-time to the Shuttle and maintains identical temperature and humidity conditions. After activation, cultures were grown for 25 hours in either spaceflight or ground until either fixation or media supplementation. Upon landing, cultures were received for processing approximately 2.5 hours after Shuttle touchdown.

Microarray analysis. Total cellular RNA purification from cultures grown in M9 media, preparation of fluorescently labeled, single stranded cDNA probes, probe hybridization to whole genome S. typhimurium microarrays, and image acquisition was performed as previously described^{1,8} using three biological and three technical replicates for each culture condition. Direct microscopic cell counting and spectrophotometric readings indicated that cell numbers in flight and ground biological replicate cultures differed by less than 2-fold. Data analysis was performed using software as described previously¹. To obtain the genes comprising the spaceflight stimulon in M9 media, the following parameters were used in Webarray software²²: an expression ratio of flight to ground of 1.8 fold or greater or 0.6 or less; a spot quality (Avalue) of greater than 9.5, and p-value of less than 0.05. To identify spaceflight stimulon genes also contained in the Hfq regulon, proteins or genes found to be regulated by Hfq or RNAs found to be bound by Hfq as reported in the indicated references were scanned against the spaceflight microarray data for expression changes within the parameters above¹¹⁻¹⁴.

Multidimensional protein identification (MudPIT) analysis via tandem mass spectrometry coupled to dual nano-liquid chromatography (LC-LC-MS/MS). Acetone-protein precipitates from whole cell lysates obtained from flight and ground cultures grown in M9 media (representing three biological replicates) were subjected to MudPIT analysis using the LC-LC-MS/MS technique (three technical replicates) as described previously^{1,23,24}. Tandem MS spectra of peptides were analyzed with TurboSEQUEST™ v 3.1 and XTandem software, and the data were further analyzed and organized using the Scaffold program^{1,23,24}. Table 6 describes the specific parameters used in Scaffold to identify the proteins in this study.

Murine infection assay. Six to eight week old female Balb/c mice (housed in the Animal Facility at the Space Life Sciences Lab at Kennedy Space Center) were deprived of food and water for approximately 6 hours and then per-orally infected with increasing dosages of S. typhimurium harvested from either flight or ground FPA cultures and resuspended in buffered saline gelatin¹. Infectious dosages increasing ten-fold in a range between approximately 1×10⁴ and 1×10⁹ bacteria (thus comprising six infectious dosages per bacterial culture) were used in the infections. Ten mice per infectious dosage were used, 20 μl per dose, and food and water were returned to the animals within 30 minutes post-infection. The infected mice were monitored every 6-12 hours for 30 days. The LD₅₀ value was calculated using the formula of Reed and Muench²⁵.

Ground based RWV cultures and acid stress assays. S. typhimurium cultures were grown in rotating wall vessels (RWVs) for 24 hours at 37 degrees C. in the LSMMG and 1×g orientations in LB, M9, or LB media supplemented with the indicated ions from M9 salts (LB-M9 salts media) and assayed for resistance to pH 3.5 as described previously^{1,15}. The percentage of surviving bacteria present after 45-60 minutes acid stress (compared to the original number of bacteria before addition of the stress) was calculated via serial dilution and CFU plating. A ratio of the percent survival values for the LSMMG and 1×g cultures in all three growth media was obtained (indicating the fold difference in survival between these cultures) and is presented as the acid survival ratio in FIG. 8. The mean and standard deviation from between two and five independent experimental trials per culture is presented with observed differences in survival ratios being statistically-significant at p-value<0.05.

### Results

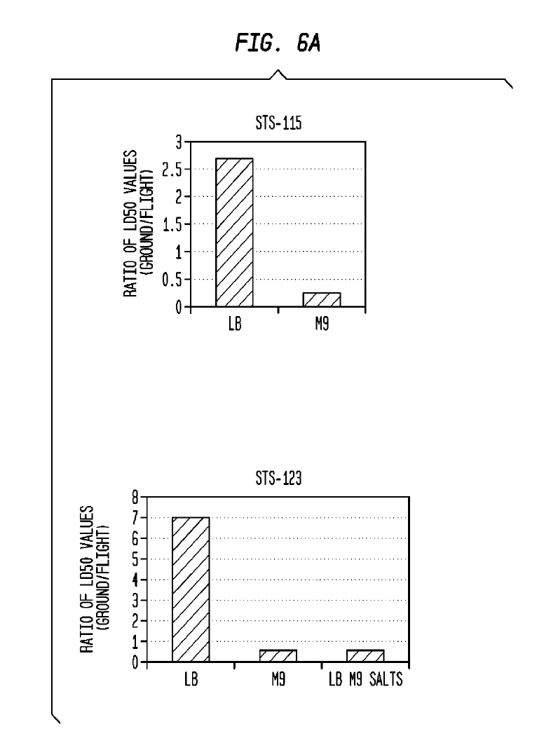

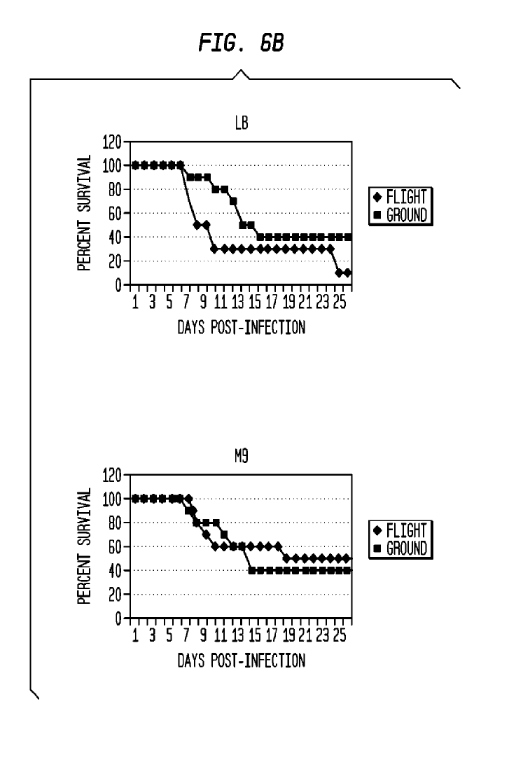

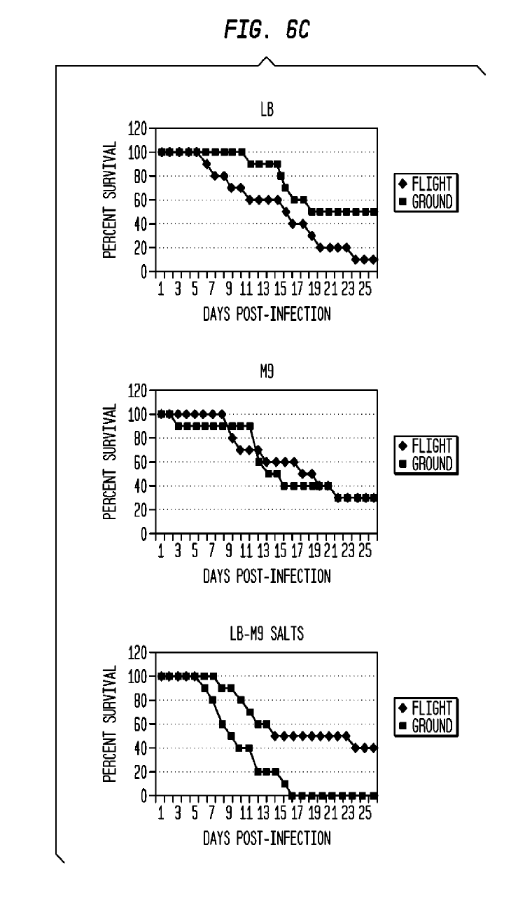

Media and virulence in spaceflight±LB. Previous flight experiments aboard STS-115 indicated that S. typhimurium cultured during spaceflight exhibited increased virulence in a murine model of infection¹. Briefly, bacteria cultured in LB during spaceflight and identical ground control cultures were harvested and immediately used to inoculate female Balb/c mice via a per-oral route of infection on the same day as Shuttle landing. Mice were infected at increasing dosages of either flight or ground cultures (10 mice per dose), and the health of the mice was monitored every 6-12 hours for 30 days. Previous results showed that mice infected with S. typhimurium grown in LB media in spaceflight aboard STS-115 displayed a decreased time to death and a 2.7 fold decrease in LD50 value compared with those infected with ground control cultures¹. To confirm these findings, the identical flight experiment was performed again aboard STS-123. In agreement with the previous experiment, mice infected with S. typhimurium grown in spaceflight aboard STS-123 displayed a decreased time to death and a 6.9 fold decrease in LD50 value compared with those infected with ground control cultures (FIG. 6, panels 6A, 6B, and 6C, LB medium).

Media and virulence in spaceflight±M9. Because of the strong association between nutrient composition of the growth media and the extent of changes observed in S. typhimurium responses in ground-based studies in the RWV, we evaluated S. typhimurium virulence using cultures grown in M9 minimal media in separate experiments aboard Space Shuttle missions STS-115 and STS-123. The procedures were otherwise identical to those described for LB media growth. However, M9 cultures from both missions displayed dramatically different virulence characteristics from those observed with LB cultured bacteria (FIGS. 6A-6C). Specifically, for infection of mice with spaceflight and ground Salmonella cultures grown in M9 media, the time to death curves overlapped and did not display the decreased time to death as seen in the LB spaceflight infections in both STS-115 and STS-123. Likewise, in contrast to observations with the LB media cultures, M9 grown cultures of S. typhimurium grown in spaceflight displayed no consistent difference in LD50 from ground controls.

To further elucidate the effect of media composition on the virulence characteristics of S. typhimurium grown during spaceflight, an additional growth medium was used that consisted of LB media supplemented with specific salts used in the preparation of M9 media. These specific salts were chosen because our quantitative trace elemental analysis showed them to be at significantly different levels in the two media. Specifically, the elemental analysis indicated that the M9 medium had dramatically higher concentrations of phosphate (61-fold higher than the LB media) and magnesium (18-fold higher than the LB media). Other notable differences in the M9 medium included higher levels of sulfate (3.6-fold higher than the LB media), chloride (3-fold higher than the LB media), and potassium (2.4-fold higher than the LB media). Thus, as follow-up flight experiment aboard STS-123, S. typhimurium virulence was evaluated using cultures grown in LB media supplemented with 25.18 mM NaH₂PO₄, 22 mM KH₂PO₄, 18.68 mM NH₄Cl, 8.54 mM NaCl, and 2 mM MgSO₄ (designated as LB-M9 salts media), thereby bringing the levels of these salts in LB media to the same as those in M9 media. Interestingly, Salmonella cultured in LB-M9 salts media displayed virulence characteristics similar to those observed when only the M9 media was used (FIGS. 6A and 6C). Specifically, as seen with cultures grown in only M9 media, mice infected with spaceflight and ground cultures grown in LB-M9 salts media did not display the decreased time to death with spaceflight grown cultures as seen in the LB infections. Also in contrast to the LB media cultures, cultures of S. typhimurium grown in LB-M9 salts media during spaceflight did not display a decreased LD50 value compared to ground controls using the same media (similar to the results with M9 media). Since nutrient composition could influence the virulence of S. typhimurium¹⁰, the LD50 values were compared for all media from flight and all media from ground controls from the STS-123 flight to highlight the effect of spaceflight on virulence (Table 4). A comparison of LD50 values from ground controls suggests that indeed media plays a role in LD50 levels, with a 5.7 fold difference between LB media and M9 media (with LB showing lower LD50 values). However, a comparison of LD50 values of cultures grown during spaceflight shows a dramatic difference approximately 10 times greater than those observed in ground cultures, as shown with a 56.8 fold difference between LB media and M9 media. This difference suggests that while media composition does affect LD50 values, the difference is exacerbated by the spaceflight environment. This indicates that there was something unique about the spaceflight environment that led to increased virulence in Salmonella.

Transcriptional and proteomic analysis. To determine which Salmonella genes changed expression in response to spaceflight culture in M9 minimal media, total bacterial RNA was isolated from fixed flight and ground samples, qualitatively analyzed to ensure lack of degradation, quantified, and then reversed transcribed into labeled, single-stranded cDNA. The labeled cDNA was co-hybridized with differentially-labeled S. typhimurium genomic DNA to whole genome S. typhimurium microarray slides. Statistically-significant differences in gene expression between the flight and ground M9 samples (above 1.8-fold increase and below 0.6-fold decrease in expression) were obtained (see Materials and Methods for details). 38 genes were found differentially-expressed in flight M9 cultures as compared to identical ground controls under these conditions (Table 5). Most notably, several genes involved in motility (9 genes: flgA, flgC, flgF, flgG, cheY, fliC, fliT, fliM, fljB), the formation of the Hyc hydrogenase (4 genes: hydN, hycF, hycD, hyB), and the Suf membrane transporter (3 genes: sufA, sufC, yhnA/sufE) were identified as differentially expressed. In addition, several genes encoding small regulatory RNA molecules (THI, csrB, rnpB, tke1) were also identified. The proteomes of fixed cultures from M9 flight and ground samples were also obtained via multi-dimensional protein identification (MudPIT) analysis. 173 proteins were identified as expressed in the flight and ground cultures, with 81 being present at statistically different levels in these samples (Table 6) indicating differential expression or stability. Notably, several proteins involved iron utilization and uptake (Fur, cytoplasmic ferritin, F—S cluster formation, bactoferrin, siderophore receptor TonB, iron transport protein, iron-dependent alcohol dehydrogenase, and ferric enterobactin receptor) and ribosome structure (L7, L32, S20, S13, S11 S19, L14, L33, S4, L4) were identified as differentially expressed. Collectively, these transcriptional and proteomic gene expression changes form the first documented bacterial spaceflight stimulon in minimal growth media.

The LB and M9 spaceflight stimulons. The S. typhimurium gene expression data from the analysis above in M9 medium were compared with the results from our previous gene expression analysis in LB medium for spaceflight and RWV cultures. Genes from each data set were cross-compared to each other to identify common genes that were present as differentially-expressed in both media. After this analysis, 15 genes (including adjacent genes) of the 38 identified as transcriptionally altered in response to spaceflight in M9 medium were also identified as differentially expressed in either spaceflight or ground-based microgravity analogue RWV culture in LB medium. This represents 39% ( 15/38) of the total genes found in the M9 transcriptional analysis.

This analysis was subsequently extended to include genes that also belong to the same directly-related functional or regulatory gene group (i.e. not necessarily the same gene or operon, but genes that function or are regulated as part of the same mechanism such as motility), and discovered that the percentage of common genes between analysis in M9 and LB media was 73% ( 28/38) (Table 5). The functional groups of genes that we identified as regulated by spaceflight or ground-based spaceflight analogue culture in both M9 and LB media included those involved in flagellar-based motility, Hyc hydrogenase formation, Suf transporter formation and other ABC transporters, and small regulatory RNA molecules (genes indicated in the section above). Additionally, there are also 8 “stand alone” genes that are believed to be not co-regulated with these gene groups and include four genes encoding putative, uncharacterized proteins (yaiA, trpD, yfiA, yhcB, grxB, acpP, yfiD, STM4002). Several genes encoding proteins identified in the spaceflight and ground proteomic analysis of M9 cultures were also identified in the gene expression analysis of M9 and LB cultures as well (Table 6).

Results from our previous studies indicated that 32% of the S. typhimurium genes identified as differentially regulated in spaceflight in LB medium belonged to a regulon of genes controlled by the conserved RNA-binding protein Hfq¹. A requirement of hfq for alterations in Salmonella acid resistance and macrophage survival was demonstrated in response to a ground-based microgravity analogue model¹. Therefore, the results of our spaceflight M9 microarray and proteomic analysis were scanned for members of a regulon of genes whose expression and activity is regulated by or regulates Hfq, or whose protein products form a functional regulatory complex with Hfq¹¹⁻¹⁴. Consistent with the previous observations in LB, four small non-coding regulatory RNA genes (THI, csrB, rnpB, tke1) and three mRNA transcripts (rpoS, sufE, fliC) regulated by Hfq were observed in the microarray analysis in M9 media (7 of 38 or 18%).

When the hits from the proteomic analysis were scanned for relationships to the Hfq regulon, 28 of the 81 proteins (34%) found to be differentially expressed in response to spaceflight in M9 media belonged to the Hfq regulon, or are part of a directly related functional group of proteins that are regulated by Hfq. Several observations led to the Hfq regulon members being highlighted in our M9 proteomic analysis: 1) Hfq promotes the expression of a large class of ribosomal structural proteins, and we found differential expression of several of these genes in spaceflight (L7/L12, L32, S20, S13, S11, S19, SA, L14, L33, S4, L4); 2) Hfq regulates the expression of the Fur protein and other genes involved in iron metabolism, and we found that Fur and other iron-related genes are differentially regulated by spaceflight in M9 medium (Fur, Dps, NifU, FepA); 3) Several other proteins encoded by genes belonging to the Hfq regulon were also found in this analysis: NmpC, Tpx, PtsI, PtsH, SucC, LeuB, CysP, DppA, OppA, RpoZ, CsrA, RpoB, NlpB. This data, taken together with the microarray data, indicates the commonalities of the spaceflight response in Salmonella in both LB and M9 media, and represents the first common genes that have been identified to be regulated by spaceflight and/or ground based spaceflight analogue culture in both rich and minimal media.

Real time PCR analysis. To further confirm the commonalities observed in global gene expression analysis in response to spaceflight in both LB and M9 media, targeted quantitative real time PCR assays were performed using cDNA synthesized from total RNA harvested from spaceflight and ground cultures in LB and M9 media as templates (FIG. 7). The csrB, yfiD, rnpB genes (down-regulated), and the trpD gene (up-regulated) were found to be differentially-regulated in response to spaceflight as compared to ground cultures in both LB and M9 media using global transcriptional analysis. These results were also found using real time PCR (FIG. 7).

Role of phosphate ion. Salmonella was previously demonstrated to consistently and reproducibly alter its acid tolerance response when grown in the RWV using LB medium¹⁵. To support findings from spaceflight that the supplementation of LB media with selected M9 salts disrupts S. typhimurium responses to this environment, cultures containing LB media, M9 media, and LB-M9 salts media were grown in the RWV at low shear modeled microgravity (LSMMG) and control orientations and evaluated for changes in acid tolerance. As demonstrated previously, cultures of S. typhimurium grown in LB media in the RWV (LSMMG) displayed altered acid resistance as compared to control cultures. However, no difference in acid tolerance was observed with cultures grown in M9 media or in LB-M9 salts media (FIG. 8). LB media supplemented with different combinations of M9 salts were then used to determine which of these ions was responsible for disruption of the acid tolerance response observed in LB medium (FIG. 8). The results indicate that the presence of phosphate from two different sources (NaH₂PO₄ and KH₂PO₄) is sufficient to disrupt the altered acid tolerance in response to LSMMG. Although hydrogen ions are present in each of these compounds, we found no correlation between the pH of the different media before or after culture and the observed phenotypes. Likewise, this indicates that the buffering capacity of phosphate is not responsible for this phenotype and that the presence of the phosphate ion itself is responsible for the acid tolerance alteration. In addition, increased osmolarity of the media is not the cause of this phenotype, since raising the level of NaCl to 25 mM (the same level as Na₂HPO₄ and KH₂PO₄) did not show the same phenotype as the presence of the phosphate-containing compounds (FIG. 8).

### Conclusion

It was found that the increased S. typhimurium virulence observed with cultures grown in spaceflight in LB medium as compared to identical ground controls is not exhibited with cultures grown in M9 medium. Based upon the quantified differences in ion concentrations between LB and M9 media, LB medium was supplemented with inorganic ions to the same levels as those found in M9 medium. This supplementation was sufficient to prevent the enhanced Salmonella virulence imparted during flight. Subsequent testing in ground-based spaceflight analogue culture conditions indicted that the altered acid tolerance exhibited by Salmonella during culture in LB alone was prevented with the addition of inorganic phosphate. These results demonstrate a direct correlation between phosphate ion concentration and the phenotypic response of Salmonella to the environment of spaceflight analogue culture. The spaceflight-induced molecular genetic responses of S. typhimurium cultured in different growth media (LB versus M9) were also compared using whole genome transcriptional and proteomic analyses. Despite the multiple phenotypic differences in response to spaceflight between the two media, several common genes and gene families were altered in expression in both media during spaceflight culture. Identification of these genes whose expression is commonly regulated by the low fluid shear environment of spaceflight provides key targets whose expression can be manipulated to control microbial responses, including use for development of vaccines and therapeutics. As identified in this study, these targets include gene systems involved in flagellar-based motility, Hyc hydrogenase formation, Suf transporter formation and other ABC transporters, ribosomal structure, iron utilization, and small regulatory RNA molecule expression and function. Many of the genes that were found differentially expressed during spaceflight culture of S. typhimurium in M9 media were also consistent with those reported in LB culture for this same organism under identical conditions. In both cases, many of these genes are found in regulons that are controlled by or regulate the activity of the Hfq protein. The findings further highlight Hfq as a global regulator to target for further study to understand the mechanism used by Salmonella to respond to spaceflight, spaceflight analogue systems, and other physiological low fluid shear environments.

TABLE 4
LD50 comparison of S. typhimurium cultured in M9 media or LB-M9
salts media relative to cultures grown only in LB media.

| Media | Growth Location | LD50 (CFU) | |
|---|---|---|---|
| | | | Media - Flight |
| | | | Fold Increase |
| | | | Relative to LB |
| LB media | Flight | 5.81 × 10⁴ | 1.0 |
| LB-M9 salts | Flight | 7.45 × 10⁵ | 12.8 |
| media | | | |
| M9 media | Flight | 3.30 × 10⁶ | 56.8 |
| | | | Fold Increase |
| | | | Relative to LB |
| | | | Media - Ground |
| LB media | Ground | 4.02 × 10⁵ | 1.0 |
| LB-M9 salts | Ground | 5.73 × 10⁵ | 1.4 |
| media | | | |
| M9 media | Ground | 2.30 × 10⁶ | 5.7 |

TABLE 5
Salmonella typhimurium genes altered in expression during growth in M9 minimal media
in spaceflight

| | Fold | Identified in | Gene | |
|---|---|---|---|---|
| STM gene | change | LB analysis* | name | Gene function |
| Up-regulated | | | | |
| STM_sRNA_THI (SEQ ID NO: | 2.69 | x | THI** | small RNA |
| 471) | | | | |
| STM0007 (SEQ ID NO: 472) | 1.91 | | talB | transaldolase B |
| STM0389 (SEQ ID NO: 473) | 1.85 | x | yaiA | putative cytoplasmic protein |
| STM1161.S (SEQ ID NO: 474) | 2.64 | | yceP | putative cytoplasmic protein |
| STM1369 (SEQ ID NO: 475) | 2.81 | x | sufA | putative HesB-like domain |
| STM1371 (SEQ ID NO: 476) | 2.65 | x | sufC | putative ABC superfamily (atp_bind) transport protein |
| STM1374 (SEQ ID NO: 477) | 1.84 | x | ynhA | putative SufE protein probably involved in Fe—S center |
| | | | | assembly |
| STM1724 (SEQ ID NO: 478) | 1.96 | x | trpD | anthranilate synthase, component II, bifunctional |
| STM2665 (SEQ ID NO: 448) | 2.53 | x | yfiA | ribosome associated factor, stabilizes ribosomes |
| | | | | against dissociation |
| STM2924 (SEQ ID NO: 479) | 2.55 | | rpoS | sigma S (sigma 38) factor of RNA polymerase |
| STM3347 (SEQ ID NO: 122) | 1.83 | x | yhcB | putative periplasmic protein |
| STM3559 (SEQ ID NO: 480) | 2.05 | | yhhV | putative cytoplasmic protein |
| STM3809.S (SEQ ID NO: 481) | 1.83 | | ibpA | small heat shock protein |
| STM4161 (SEQ ID NO: 482) | 2.00 | | | putative involved in thiamine biosynthesis |
| Down-regulated | | | | |
| STM_PSLT014 (SEQ ID NO: | 0.52 | | orf6 | putative outer membrane protein |
| 483) | | | | |
| STM_sRNA_CsrB (SEQ ID | 0.51 | x | csrB | regulatory RNA |
| NO: 84) | | | | |
| STM_sRNA_RNaseP (SEQ ID | 0.44 | x | rnpB | regulatory RNA |
| NO: 484) | | | | |
| STM_sRNA_tke1 (SEQ ID NO: | 0.58 | x | tke1 | small RNA |
| 440) | | | | |
| STM1078 (SEQ ID NO: 485) | 0.43 | | | putative cytoplasmic protein |
| STM1165 (SEQ ID NO: 486) | 0.57 | x | grxB | glutaredoxin 2 |
| STM1173 (SEQ ID NO: 487) | 0.57 | x | flgA | flagellar biosynthesis; assembly of basal-body |
| | | | | periplasmic P ring |
| STM1175 (SEQ ID NO: 488) | 0.37 | x | flgC | flagellar biosynthesis, cell-proximal portion of basal- |
| | | | | body rod |
| STM1178 (SEQ ID NO: 489) | 0.52 | x | flgF | flagellar biosynthesis, cell-proximal portion of basal- |
| | | | | body rod |
| STM1179 (SEQ ID NO: 490) | 0.47 | x | flgG | flagellar biosynthesis, cell-distal portion of basal-body |
| | | | | rod |
| STM1196 (SEQ ID NO: 134) | 0.59 | x | acpP | acyl carrier protein |
| STM1466 (SEQ ID NO: 491) | 0.59 | | ydgA | putative periplasmic protein |
| STM1916 (SEQ ID NO: 76) | 0.55 | x | cheY | chemotaxis regulator, transmits chemoreceptor signals |
| | | | | to flagelllar motor |
| STM1959 (SEQ ID NO: 1) | 0.44 | x | fliC | flagellar biosynthesis; flagellin, filament structural |
| | | | | protein |
| STM1962 (SEQ ID NO: 78) | 0.54 | x | fliT | flagellar biosynthesis; possible export chaperone for |
| | | | | FliD |
| STM1976 (SEQ ID NO: 492) | 0.59 | x | fliM | flagellar biosynthesis, component of motor switch and |
| | | | | energizing |
| STM2646 (SEQ ID NO: 141) | 0.44 | x | yfiD | putative formate acetyltransferase |
| STM2771 (SEQ ID NO: 493) | 0.31 | x | fljB | Flagellar synthesis: phase 2 flagellin (filament structural |
| | | | | protein) |
| STM2843 (SEQ ID NO: 26) | 0.49 | x | hydN | electron transport protein (FeS senter) from formate to |
| | | | | hydrogen |
| STM2848 (SEQ ID NO: 494) | 0.59 | x | hycF | hydrogenase 3, putative quinone oxidoreductase |
| STM2850 (SEQ ID NO: 495) | 0.59 | x | hycD | hydrogenase 3, membrane subunit (part of FHL |
| | | | | complex) |
| STM2852 (SEQ ID NO: 496) | 0.52 | x | hycB | hydrogenase-3, iron-sulfur subunit (part of FHL |
| | | | | complex) |
| STM4002 (SEQ ID NO: 162) | 0.53 | x | | putative cytoplasmic protein |
| STM4063 (SEQ ID NO: 497) | 0.55 | | sbp | ABC superfamily (bind_prot), sulfate transport protein |

*Genes, operons, or directly-related functional groups identified as also being differnetially-regulated during growth in spaceflight or ground-based modeled microgravity in LB medium
**STM genome coordinates: 4382782-4382542

TABLE 6
Salmonella typhimurium proteins identified via MudPit analysis as present
during growth in M9 minimal media in spaceflight (173 proteins total)

| | | Protein | Flight | Ground |
|---|---|---|---|---|
| | | molecular | total cell | total cell |
| | Accession | weight | protein ID | protein ID |
| Protein name | number | (Daltons) | probability* | probability* |
| sn-glycerol-3-phosphate dehydrogenase | gi\|16766813 (SEQ ID NO: 187) | 57 kDa | 100% (100%) | 99% (99%) |
| branched-chain-amino-acid transaminase | gi\|96710 (SEQ ID NO: 498) | 34 kDa | 99% (99%) | 100% (100%) |
| putative periplasmic protein | gi\|16764930 (SEQ ID NO: 499) | 39 kDa | 100% (100%) | 100% (100%) |
| 6,7-dimethyl-8-ribityllumazine synthase | gi\|16501685 (SEQ ID NO: 500) | 16 kDa | 100% (100%) | 100% (100%) |
| thioredoxin reductase | gi\|16502122 (SEQ ID NO: 501) | 35 kDa | 100% (100%) | 100% (100%) |
| 50S ribosomal subunit protein L2 | gi\|16505152 (SEQ ID NO: 502) | 30 kDa | 100% (100%) | 100% (100%) |
| 30S ribosomal protein S10 | gi\|68057571 (SEQ ID NO: 503) | 12 kDa | 100% (100%) | 100% (100%) |
| 50S ribosomal protein L24 | gi\|15803836 (SEQ ID NO: 504) | 11 kDa | 100% (100%) | 100% (100%) |
| serine hydroxymethyltransferase | gi\|16503768 (SEQ ID NO: 505) | 45 kDa | 100% (100%) | 100% (100%) |
| 30s ribosomal protein S6 | gi\|16505516 (SEQ ID NO: 506) | 15 kDa | 100% (100%) | 100% (100%) |
| 30S ribosomal protein S22 | gi\|16502603 (SEQ ID NO: 507) | 5 kDa | 100% (100%) | 100% (100%) |
| 2,3,4,5-tetrahydropyridine-2-carboxylate N- | gi\|16763603 (SEQ ID NO: 508) | 30 kDa | 100% (100%) | 100% (100%) |
| succinyltransferase | | | | |
| peptidyl-prolyl cis-trans isomerase B | gi\|16501803 (SEQ ID NO: 509) | 18 kDa | 100% (100%) | 100% (100%) |
| FKBP-type peptidyl-prolyl cis-trans isomerase | gi\|16766744 (SEQ ID NO: 277) | 21 kDa | 100% (100%) | 100% (100%) |
| ribosome recycling factor | gi\|16501500 (SEQ ID NO: 510) | 21 kDa | 100% (100%) | 100% (100%) |
| carbamoyl-phosphate synthase large subunit | gi\|16763457 (SEQ ID NO: 511) | 118 kDa | 100% (100%) | 100% (100%) |
| ATP synthase delta subunit | gi\|16504763 (SEQ ID NO: 512) | 19 kDa | 100% (100%) | 100% (100%) |
| FKBP-type peptidyl-prolyl cis-trans isomerase | gi\|16766742 (SEQ ID NO: 513) | 29 kDa | 99% (99%) | 100% (100%) |
| putative cytoplasmic protein | gi\|56383221 (SEQ ID NO: 514) | 12 kDa | 99% (99%) | 100% (100%) |
| D-3-phosphoglycerate dehydrogenase | gi\|16766363 (SEQ ID NO: 515) | 44 kDa | 100% (100%) | 100% (100%) |
| 50S ribosomal subunit protein L3 | gi\|16505149 (SEQ ID NO: 516) | 22 kDa | 100% (100%) | 100% (100%) |
| ATP synthase alpha subunit | gi\|16504764 (SEQ ID NO: 517) | 55 kDa | 100% (100%) | 100% (100%) |
| serine endoprotease | gi\|16766643 (SEQ ID NO: 361) | 47 kDa | 100% (100%) | 100% (100%) |
| putative ABC-type transport system ATPase | gi\|16763890 (SEQ ID NO: 518) | 30 kDa | 99% (99%) | 100% (100%) |
| ATP synthase subunit B | gi\|16504762 (SEQ ID NO: 519) | 17 kDa | 99% (99%) | 100% (100%) |
| malate dehydrogenase | gi\|16766654 (SEQ ID NO: 238) | 32 kDa | 99% (99%) | 100% (100%) |
| RNase E | gi\|16764541 (SEQ ID NO: 313) | 119 kDa | 100% (100%) | 100% (100%) |
| osmotically inducible protein C | gi\|16502605 (SEQ ID NO: 520) | 15 kDa | 99% (99%) | 100% (100%) |
| polynucleotide phosphorylase | gi\|16766580 (SEQ ID NO: 236) | 77 kDa | 100% (100%) | 100% (100%) |
| glutamate dehydrogenase | gi\|16764650 (SEQ ID NO: 521) | 49 kDa | 100% (100%) | 100% (100%) |
| menaquinone biosynthesis protein | gi\|16504650 (SEQ ID NO: 522) | 17 kDa | 99% (99%) | 100% (100%) |
| 50S ribosomal subunit protein L18 | gi\|16505166 (SEQ ID NO: 523) | 13 kDa | 99% (99%) | 100% (100%) |
| phospho-2-dehydro-3-deoxyheptonate aldolase | gi\|16503824 (SEQ ID NO: 524) | 39 kDa | 99% (99%) | 100% (100%) |
| transketolase | gi\|16766377 (SEQ ID NO: 199) | 72 kDa | 99% (99%) | 100% (100%) |
| acetylglutamate kinase | gi\|16767387 (SEQ ID NO: 525) | 27 kDa | 99% (99%) | 100% (100%) |
| 50S ribosomal protein L11 | gi\|15804573 (SEQ ID NO: 526) | 15 kDa | 99% (99%) | 100% (100%) |
| cold shock protein CspC | gi\|15802236 (SEQ ID NO: 527) | 7 kDa | 100% (100%) | 100% (100%) |
| unnamed protein product | gi\|47736 (SEQ ID NO: 528) | 16 kDa | 100% (100%) | 100% (100%) |
| dihydrolipoamide dehydrogenase | gi\|16763544 (SEQ ID NO: 186) | 51 kDa | 100% (100%) | 100% (100%) |
| 50S ribosomal subunit protein L29 | gi\|16505157 (SEQ ID NO: 260) | 7 kDa | 100% (100%) | 100% (100%) |
| arginine-binding periplasmic protein 1 precursor | gi\|16502093 (SEQ ID NO: 529) | 27 kDa | 100% (100%) | 100% (100%) |
| 50S ribosomal subunit protein L10 | gi\|47916 (SEQ ID NO: 530) | 18 kDa | 100% (100%) | 100% (100%) |
| hyperosmotically-inducible periplasmic protein | gi\|16505665 (SEQ ID NO: 531) | 21 kDa | 100% (100%) | 100% (100%) |
| bacterioferritin comigratory protein | gi\|16503708 (SEQ ID NO: 532) | 18 kDa | 100% (100%) | 100% (100%) |
| glutamate/aspartate transporter | gi\|16764042 (SEQ ID NO: 533) | 34 kDa | 100% (100%) | 100% (100%) |
| 50S ribosomal subunit protein L17 | gi\|16505176 (SEQ ID NO: 534) | 14 kDa | 100% (100%) | 100% (100%) |
| 50S ribosomal subunit protein L6 | gi\|16505165 (SEQ ID NO: 535) | 19 kDa | 100% (100%) | 100% (100%) |
| fructose 1,6-bisphosphate aldolase | gi\|16504152 (SEQ ID NO: 536) | 39 kDa | 100% (100%) | 100% (100%) |
| 30S ribosomal protein S3 | gi\|16131193 (SEQ ID NO: 537) | 26 kDa | 100% (100%) | 100% (100%) |
| 5-methyltetrahydropteroyltriglutamate--homocysteine | gi\|16767235 (SEQ ID NO: 538) | 85 kDa | 100% (100%) | 100% (100%) |
| methyltransferase | | | | |
| phosphate acetyltransferase | gi\|16765665 (SEQ ID NO: 279) | 77 kDa | 99% (99%) | 100% (100%) |
| inorganic pyrophosphatase | gi\|16505542 (SEQ ID NO: 539) | 20 kDa | 100% (100%) | 100% (100%) |
| phase 1 flagellin | gi\|50830926 (SEQ ID NO: 540) | 52 kDa | 99% (99%) | 100% (100%) |
| histone like DNA-binding protein HU-alpha | gi\|16504591 (SEQ ID NO: 541) | 10 kDa | 100% (100%) | 100% (100%) |
| (NS2) (HU-2) | | | | |
| Iron transport protein, periplasmic-binding protein | gi\|16503939 (SEQ ID NO: 542) | 34 kDa | 100% (100%) | 100% (100%) |
| conserved hypothetical protein | gi\|16503804 (SEQ ID NO: 543) | 14 kDa | 100% (100%) | 100% (100%) |
| ribosomal protein S7 | gi\|47922 (SEQ ID NO: 544) | 18 kDa | 100% (100%) | 100% (100%) |
| ATP synthase beta subunit | gi\|16504766 (SEQ ID NO: 545) | 50 kDa | 100% (100%) | 100% (100%) |
| 30S ribosomal protein S2 | gi\|16501497 (SEQ ID NO: 546) | 27 kDa | 100% (100%) | 100% (100%) |
| glucose-specific PTS system enzyme IIA component | gi\|47658 (SEQ ID NO: 547) | 18 kDa | 100% (100%) | 100% (100%) |
| probable peroxidase | gi\|16501671 (SEQ ID NO: 548) | 22 kDa | 100% (100%) | 100% (100%) |
| 50S ribosomal subunit protein L5 | gi\|16505162 (SEQ ID NO: 549) | 20 kDa | 100% (100%) | 100% (100%) |
| argininosuccinate lyase | gi\|16767388 (SEQ ID NO: 550) | 50 kDa | 100% (100%) | 100% (100%) |
| RNA polymerase, alpha subunit | gi\|24053769 (SEQ ID NO: 551) | 37 kDa | 100% (100%) | 100% (100%) |
| glycine/betaine/proline transport protein | gi\|16766122 (SEQ ID NO: 552) | 36 kDa | 100% (100%) | 100% (100%) |
| elongation factor Ts | gi\|16501498 (SEQ ID NO: 553) | 30 kDa | 100% (100%) | 100% (100%) |
| inositol-5-monophosphate dehydrogenase | gi\|16765831 (SEQ ID NO: 338) | 52 kDa | 100% (100%) | 100% (100%) |
| putative outer membrane lipoprotein | gi\|16763634 (SEQ ID NO: 554) | 29 kDa | 100% (100%) | 100% (100%) |
| DNA-directed RNA polymerase, beta-subunit | gi\|16504603 (SEQ ID NO: 555) | 155 kDa | 100% (100%) | 100% (100%) |
| GroEL protein | gi\|16505460 (SEQ ID NO: 556) | 57 kDa | 100% (100%) | 100% (100%) |
| enolase | gi\|16504025 (SEQ ID NO: 557) | 46 kDa | 100% (100%) | 100% (100%) |
| glyceraldehyde 3-phosphate dehydrogenase A | gi\|16502901 (SEQ ID NO: 558) | 36 kDa | 100% (100%) | 100% (100%) |
| translation elongation factor EF-Tu.A | gi\|96718 (SEQ ID NO: 169) | 43 kDa | 100% (100%) | 100% (100%) |
| iron-dependent alcohol dehydrogenase | gi\|16765093 (SEQ ID NO: 193) | 96 kDa | 100% (100%) | 100% (100%) |
| phosphoglycerate kinase | gi\|16504153 (SEQ ID NO: 559) | 41 kDa | 100% (100%) | 100% (100%) |
| putative hydrogenase membrane component precurosr | gi\|16764429 (SEQ ID NO: 171) | 38 kDa | 100% (100%) | 100% (100%) |
| formate acetyltransferase 1 | gi\|16502136 (SEQ ID NO: 560) | 85 kDa | 100% (100%) | 100% (100%) |
| 30S ribosomal protein S1 | gi\|16502144 (SEQ ID NO: 561) | 61 kDa | 100% (100%) | 100% (100%) |
| elongation factor G | gi\|47923 (SEQ ID NO: 562) | 78 kDa | 100% (100%) | 100% (100%) |
| 30S ribosomal subunit protein S5 | gi\|24053777 (SEQ ID NO: 563) | 18 kDa | 100% (100%) | 100% (100%) |
| O-Acetylserine Sulfhydrylase | gi\|11514514 (SEQ ID NO: 270) | 34 kDa | 100% (100%) | 100% (100%) |
| trigger factor | gi\|16501718 (SEQ ID NO: 564) | 48 kDa | 100% (100%) | 100% (100%) |
| Glutamine Synthetase | gi\|9256972 (SEQ ID NO: 565) | 52 kDa | 100% (100%) | 100% (100%) |
| molecular chaperone DnaK | gi\|16763402 (SEQ ID NO: 179) | 69 kDa | 100% (100%) | 100% (100%) |
| arginine-binding periplasmic protein 2 precursor | gi\|16502090 (SEQ ID NO: 566) | 27 kDa | 100% (100%) | 100% (100%) |
| alkyl hydroperoxide reductase c22 protein | gi\|16501859 (SEQ ID NO: 567) | 21 kDa | 100% (100%) | 100% (100%) |
| 50S ribosomal protein L9 | gi\|16767640 (SEQ ID NO: 216) | 16 kDa | 100% (100%) | 100% (100%) |
| outer membrane protein OmpH precursor | gi\|16501506 (SEQ ID NO: 568) | 18 kDa | 100% (100%) | 100% (100%) |
| glutamine-binding periplasmic protein precursor | gi\|16502041 (SEQ ID NO: 569) | 27 kDa | 100% (100%) | 100% (100%) |
| GroES protein | gi\|16505459 (SEQ ID NO: 570) | 10 kDa | 100% (100%) | 100% (100%) |
| 50S ribosomal subunit protein L1 | gi\|16504607 (SEQ ID NO: 571) | 25 kDa | 100% (100%) | 100% (100%) |
| outer membrane protein C | gi\|16503494 (SEQ ID NO: 572) | 41 kDa | 100% (100%) | 100% (100%) |
| dipeptide transport protein | gi\|16766917 (SEQ ID NO: 352) | 60 kDa | 100% (100%) | |
| PTS system protein HPr | gi\|24052838 (SEQ ID NO: 573) | 9 kDa | 100% (100%) | |
| 50S ribosomal subunit protein L7/L12 | gi\|47917 (SEQ ID NO: 574) | 12 kDa | 100% (100%) | |
| 50S ribosomal subunit protein L32 | gi\|24051382 (SEQ ID NO: 575) | 6 kDa | 100% (100%) | |
| oligopeptide transport protein | gi\|39546324 (SEQ ID NO: 576) | 61 kDa | 100% (100%) | |
| high-affinity branched-chain amino acid transporter | gi\|16766853 (SEQ ID NO: 577) | 39 kDa | 100% (100%) | |
| 30S ribosomal protein S20 | gi\|16501327 (SEQ ID NO: 578) | 10 kDa | 100% (100%) | |
| phosphoribosylaminoimidazole carboxylase catalytic | gi\|16763914 (SEQ ID NO: 579) | 18 kDa | 100% (100%) | |
| subunit | | | | |
| putative translation initiation inhibitor | gi\|16505567 (SEQ ID NO: 580) | 14 kDa | 100% (100%) | |
| putative multicopper oxidase | gi\|16763558 (SEQ ID NO: 581) | 59 kDa | 100% (100%) | |
| DNA protection during starvation protein | gi\|16502042 (SEQ ID NO: 582) | 19 kDa | 100% (100%) | |
| thioredoxin | gi\|67005950 (SEQ ID NO: 583) | 12 kDa | 100% (100%) | |
| sulfate transport protein | gi\|16767329 (SEQ ID NO: 584) | 37 kDa | 100% (100%) | |
| ribulose-phosphate 3-epimerase | gi\|16766771 (SEQ ID NO: 585) | 24 kDa | 99% (99%) | |
| cytoplasmic ferritin | gi\|16765276 (SEQ ID NO: 258) | 19 kDa | 99% (99%) | |
| osmotically inducible lipoprotein E precursor | gi\|16502880 (SEQ ID NO: 586) | 12 kDa | 100% (100%) | |
| fructose-bisphosphate aldolase class I | gi\|16503381 (SEQ ID NO: 587) | 38 kDa | 100% (100%) | |
| 30S ribosomal protein S13 | gi\|16766707** (SEQ ID NO: 202) | 13 kDa | 100% (100%) | |
| thiosulfate transport protein | gi\|16765764 (SEQ ID NO: 588) | 38 kDa | 100% (100%) | |
| histidine-binding periplasmic protein | gi\|47731 (SEQ ID NO: 589) | 28 kDa | 100% (100%) | |
| RecA protein | gi\|16503906 (SEQ ID NO: 590) | 38 kDa | 100% (100%) | |
| aspartate semialdehyde dehydrogenase | gi\|2353187 (SEQ ID NO: 591) | 43 kDa | 100% (100%) | |
| transcription elongation factor NusA | gi\|16766585** (SEQ ID NO: 245) | 55 kDa | 100% (100%) | |
| pyruvate kinase | gi\|16764728 (SEQ ID NO: 220) | 51 kDa | 99% (99%) | |
| 30S ribosomal protein S11 | gi\|16766706** (SEQ ID NO: 265) | 14 kDa | 99% (99%) | |
| DNA-directed RNA polymerase omega subunit | gi\|15804190 (SEQ ID NO: 592) | 10 kDa | 99% (99%) | |
| single-strand DNA-binding protein | gi\|16505243 (SEQ ID NO: 593) | 19 kDa | 99% (99%) | |
| DNA-binding protein HU-beta | gi\|581767 (SEQ ID NO: 594) | 9 kDa | 99% (99%) | |
| putative cytoplasmic protein | gi\|6851082 (SEQ ID NO: 595) | 19 kDa | 99% (99%) | |
| succinyl-CoA synthetase beta chain | gi\|16501970 (SEQ ID NO: 596) | 41 kDa | 99% (99%) | |
| 50S ribosomal subunit protein A | gi\|56383471 (SEQ ID NO: 597) | 7 kDa | 99% (99%) | |
| ATPase subunit | gi\|7594817 (SEQ ID NO: 328) | 46 kDa | 99% (99%) | |
| ketol-acid reductoisomerase | gi\|16767185 (SEQ ID NO: 598) | 54 kDa | 98% (98%) | |
| DNA ligase | gi\|16765747 (SEQ ID NO: 599) | 73 kDa | 99% (99%) | |
| 30S ribosomal subunit protein S19 | gi\|16505153 (SEQ ID NO: 600) | 10 kDa | 100% (100%) | |
| NifU-like protein involved in Fe—S cluster formation | gi\|16503756 (SEQ ID NO: 601) | 14 kDa | 99% (99%) | |
| putative sigma(54) modulation protein | gi\|16503819 (SEQ ID NO: 602) | 13 kDa | 99% (99%) | |
| 3-isopropylmalate dehydrogenase | gi\|16763502** (SEQ ID NO: 603) | 40 kDa | 99% (99%) | |
| bacterioferrin | gi\|16766732** (SEQ ID NO: 604) | 18 kDa | 99% (99%) | |
| glycerate kinase II | gi\|16763905** (SEQ ID NO: 326) | 39 kDa | 99% (99%) | |
| DNA polymerase I | gi\|16767264 (SEQ ID NO: 325) | 103 kDa | 99% (99%) | |
| putative periplasmic protein | gi\|16764812 (SEQ ID NO: 605) | 54 kDa | 99% (99%) | |
| threonine dehydratase | gi\|16767181 (SEQ ID NO: 606) | 56 kDa | 98% (98%) | |
| putative universal stress protein UspA | gi\|16501866 (SEQ ID NO: 607) | 16 kDa | 99% (99%) | |
| ferric uptake regulator Fur | gi\|16501929 (SEQ ID NO: 608) | 17 kDa | 99% (99%) | |
| Cell division protease ftsH | gi\|16504361 (SEQ ID NO: 609) | 71 kDa | 99% (99%) | |
| flavodoxin | gi\|16764064 (SEQ ID NO: 610) | 20 kDa | 99% (99%) | |
| nitrate reductase 2 beta subunit | gi\|16764922 (SEQ ID NO: 611) | 59 kDa | 99% (99%) | |
| keto-hydroxyglutarate-aldolase/keto-deoxy- | gi\|16765226 (SEQ ID NO: 612) | 22 kDa | 99% (99%) | |
| phosphogluconate aldolase | | | | |
| carbon storage regulator CsrA | gi\|24053109 (SEQ ID NO: 613) | 7 kDa | 99% (99%) | |
| rmlC dTDP-4,deoxyrhamnose 3,5 epimerase | gi\|581655 (SEQ ID NO: 614) | 21 kDa | 99% (99%) | |
| TonB-dependent siderophore receptor protein | gi\|16766089 (SEQ ID NO: 615) | 79 kDa | | 100% |
| N-succinyldiaminopimelate- | gi\|16766756 (SEQ ID NO: 616) | 44 kDa | | 100% |
| aminotransferase/acetylornithine transaminase | | | | |
| DNA-directed RNA polymerase beta subunit | gi\|16767407 (SEQ ID NO: 235) | 151 kDa | | 100% |
| argininosuccinate synthase | gi\|39546365 (SEQ ID NO: 617) | 50 kDa | | 100% |
| outer membrane lipoprotein SlyB precursor | gi\|16502764 (SEQ ID NO: 618) | 16 kDa | | 100% |
| thiol peroxidase | gi\|16765025 (SEQ ID NO: 619) | 18 kDa | | 100% |
| 50S ribosomal subunit protein L14 | gi\|49613467 (SEQ ID NO: 620) | 14 kDa | | 100% |
| triosephosphate isomerase | gi\|16767347 (SEQ ID NO: 210) | 27 kDa | | 100% |
| lipoprotein | gi\|16765808** (SEQ ID NO: 216) | 37 kDa | | 100% |
| oligopeptidase A | gi\|1676688** (SEQ ID NO: 621) | 77 kDa | | 100% |
| phosphoribosylamine--glycine ligase | gi\|16767429 (SEQ ID NO: 622) | 46 kDa | | 100% |
| phosphoglyceromutase | gi\|16764136 (SEQ ID NO: 623) | 28 kDa | | 100% |
| cystathionine gamma-synthase | gi\|16767366 (SEQ ID NO: 624) | 42 kDa | | 100% |
| glucose-6-phosphate isomerase | gi\|16767471 (SEQ ID NO: 281) | 61 kDa | | 100% |
| 50S ribosomal subunit protein L33 | gi\|24054147 (SEQ ID NO: 625) | 6 kDa | | 100% |
| ribosomal protein S4 | gi\|2780215 (SEQ ID NO: 626) | 23 kDa | | 100% |
| PEP-protein phosphotransferase | gi\|16765752** (SEQ ID NO: 627) | 63 kDa | | 100% |
| Lpp1 major outer membrane lipoprotein | gi\|37785814 (SEQ ID NO: 628) | 8 kDa | | 100% |
| aldose 1-epimerase | gi\|16764640** (SEQ ID NO: 287) | 33 kDa | | 100% |
| putative outer membrane porin precursor | gi\|16764916** (SEQ ID NO: 197) | 40 kDa | | 99% |
| 50S ribosomal protein L4 | gi\|15803846 (SEQ ID NO: 629) | 22 kDa | | 100% |
| outer membrane ferric enterobactin receptor precursor | gi\|16763962 (SEQ ID NO: 630) | 83 kDa | | 100% |
| hypothetical protein STM2795 putative LysM domain | gi\|16766106** (SEQ ID NO: 631) | 16 kDa | | 100% |
| phosphoribosylaminoimidazole-succinocarboxamide | gi\|16503704 (SEQ ID NO: 632) | 27 kDa | | 100% |
| synthase | | | | |
| arnithine carbamoyltransferase | gi\|312706 (SEQ ID NO: 633) | 24 kDa | | 100% |
| ribonucleoside-diphosphate reductase | gi\|1184247 (SEQ ID NO: 634) | 81 kDa | | 100% |
| biotin carboxylase | gi\|16504442 (SEQ ID NO: 635) | 49 kDa | | 100% |
| 3-ketoacyl-(acyl-carrier-protein) reductase | gi\|16764550 (SEQ ID NO: 636) | 26 kDa | | 100% |
| 6-phosphofructokinase II | gi\|16764677 (SEQ ID NO: 637) | 33 kDa | | 100% |
| uroporphyrinogen III methylase | gi\|16767207 (SEQ ID NO: | 42 kDa | | 100% |
| | 638) | | | |

*Peptide samples obtained from MudPIT were analyzed using Sequest and X!Tandem software, and the data was organized using the Scaffold program. To be considered a positive identification in Scaffold, the following parameters were used: a minimum of 2 peptides from a given protein identified with peptide and protein thresholds of 80% to give an overall protein identification (ID) probability of at least 80%. Note that a protein ID probability of greater than 80% in at least one of the samples warranted inclusion in the table so as to allow identification of possible differential expression of a given protein.
**Peptides encoded by genes or operons also found to be differentially regulated in spaceflight or ground based modeled microgravity in LB medium

### REFERENCES

1. Wilson, J. W. et al. Space flight alters bacterial gene expression and virulence and reveals a role for global regulator Hfq. Proc Natl Acad Sci USA 104 (41), 16299-16304 (2007).
2. Aertsen, A & Michiels, CW Stress and how bacteria cope with death and survival. Crit Rev Microbiol 30, 263-273 (2004).
3. Rychlik, I. & Barrow, P. A. Salmonella stress management and its relevance to behaviour during intestinal colonisation and infection. FEMS Microbiol Rev 29 (5), 1021-1040 8 (2005).
4. Foster, J. W. Escherichia coli acid resistance: tales of an amateur acidophile. Nat Rev Microbiol 2 (11), 898-907 (2004).
5. Beeson, J. G. et al. Adhesion of Plasmodium falciparum-infected erythrocytes to hyaluronic acid in placental malaria. Nat Med 6 (1), 86-90 (2000).
6. Cai, Z., Xin, J., Pollock, D. M., & Pollock, J. S. Shear stress-mediated NO production in inner medullary collecting duct cells. Am J Physiol Renal Physiol 279 (2), F270-274 15 (2000).
7. Guo, P., Weinstein, A. M., & Weinbaum, S. A hydrodynamic mechanosensory hypothesis for brush border microvilli. Am J Physiol Renal Physiol 279 (4), F698-712 18 (2000).
8. Wilson, J. W. et al. Microarray analysis identifies Salmonella genes belonging to the low-shear modeled microgravity regulon. Proc Natl Acad Sci USA 99 (21), 1380721 13812 (2002).
9. Wilson, J. W. et al. Low-Shear modeled microgravity alters the Salmonella enterica serovar typhimurium stress response in an RpoS-independent manner. Appl Environ 24 Microbiol 68 (11), 5408-5416 (2002).
10. Adkins, J. N. et al. Analysis of the Salmonella typhimurium Proteome through Environmental Response toward Infectious Conditions. Mol Cell Proteomics 5 (8), 145027 1461 (2006).
11. Figueroa-Bossi, N. et al. Loss of Hfq activates the sigmaE-dependent envelope stress response in Salmonella enterica. Mol Microbiol 62 (3), 838-852 (2006).
12. Guisbert, E. et al. Hfq modulates the sigmaE-mediated envelope stress response and the sigma-mediated cytoplasmic stress response in Escherichia coli. J Bacteriol 189 (5), 32 1963-1973 (2007).
13. Sittka, A., Pfeiffer, V., Tedin, K., & Vogel, J. The RNA chaperone Hfq is essential for the virulence of Salmonella typhimurium. Mol Microbiol 63 (1), 193-217 (2007).
14. Zhang, A. et al. Global analysis of small RNA and mRNA targets of Hfq. Mol Microbiol 50 (4), 1111-1124 (2003).
15. Nickerson, C. A. et al. Microgravity as a novel environmental signal affecting Salmonella enterica serovar Typhimurium virulence. Infect Immun 68 (6), 3147-3152 (2000).
16. Ruiz, Natividad & Silhavy, Thomas J. Constitutive Activation of the Escherichia coli Pho Regulon Upregulates rpoS Translation in an Hfq-Dependent Fashion. J. Bacteriol. 185 41 (20), 5984-5992 (2003).
17. Vanderpool, C. K. & Gottesman, S. Involvement of a novel transcriptional activator and small RNA in post-transcriptional regulation of the glucose phosphoenolpyruvate phosphotransferase system. Mol Microbiol 54 (4), 1076-1089 (2004).
18. Gulig, P. A. & Curtiss, R., 3rd Plasmid-associated virulence of Salmonella typhimurium. Infect Immun 55 (12), 2891-2901 (1987).
19. Lennox, E. S. Transduction of linked genetic characters of the host by bacteriophage P1. Virology 1 (2), 190-206 (1955).
20. Crabbe, A. et al. Use of the rotating wall vessel technology to study the effect of shear stress on growth behaviour of Pseudomonas aeruginosa PA01. Environ Microbiol (2008).
21. ASTM Method D4327, Standard Test Method for Anions in Water by Chemically Suppressed Ion Chromatography. ASTM Method D4327, Standard Test Method for Anions in Water by Chemically Suppressed Ion Chromatography, ASTM International, West Conshohocken, Pa., www.astm.org.
22. Xia, X, McClelland, M, & Wang, Y WebArray: an online platform for microarray data analysis. BMC Bioinformatics 6, 306 (2005).
23. Keller, A., Nesvizhskii, A. I., Kolker, E., & Aebersold, R. Empirical statistical model to estimate the accuracy of peptide identifications made by MS/MS and database search. Anal Chem 74 (20), 5383-5392 (2002).
24. Nesvizhskii, A. I., Keller, A., Kolker, E., & Aebersold, R. A statistical model for identifying proteins by tandem mass spectrometry. Anal Chem 75 (17), 4646-4658 (2003).
25. Reed, L. J. & Muench, H. A simple method of estimating fifty percent endpoints. Am J Hyg 27, 493-497 (1938).

### Example 3

This example describes a general protocol for culturing a live attenuated Salmonella enterica serovar Typhimurium vaccine strain under low sedimental shear conditions, and to evaluate the immunogenicity of the vaccine strain cultured in this manner in a mouse model. A recombinant attenuated Salmonella enterica serovar Typhimurium anti-pneumococcal vaccine strain x9558 (Δpmi-2426 Δ(gmd-fcl)-26 ΔPfur₃₃::TTaraCP_{BAD}fur ΔPcrp₅₂₇::TTaraCP_{BAD}crp δasdA27::TTaraCP_{BADc2} ΔaraE25 ΔaraBAD23 ΔrelA198::araCP_{BAD}lacITT ΔsopB1925 ΔagfBAC811 ΔfliC180 ΔfljB217) encoding pneumococcal antigen (pspA capsular gene) on plasmid pYA4088 is used in this example as illustration. However, any live attenuated bacterial vaccine strain can be used that carries one or more attenuating mutations of interest—including heterologous recombinant vaccine strains that express foreign antigens to elicit innate humoral and cellular immune responses. Moreover, Lennox broth is used for Salmonella strain culture in this example, any growth media and incubation conditions required to cultivate the strain of interest can be used. In addition, while the Rotating Wall Vessel bioreactor is used as the culture modality to achieve low sedimental shear stress, other culture environments that achieve this environment can also be used (including the spaceflight environment).

Live attenuated bacterial vaccine strain growth conditions. The attenuated Salmonella vaccine strain is first grown in Lennox broth (L-broth) as a static or aerated overnight culture at 37° C. Cultures are then inoculated at a dilution of 1:200 into 50 ml of L broth and subsequently introduced into the RWV bioreactor. Care is taken to ensure that the reactor is completely filled with culture media and no bubbles are present (i.e. zero headspace). The reactor vessel is oriented to grow cells under conditions of low sedimental shear or control sedimental shear. Two different RWV bioreactors, one in each physical orientation (low sedimental shear or control sedimental shear, respectively), should be simultaneously inoculated with the bacterial strain. Incubations in the RWV are at 37° C. or room temperature with a rotation rate of 25 rpm. Culture times are for 10 hours (which corresponds to mid-log phase growth) or 24 hours (which corresponds to stationary phase). Cell density is measured as viable bacterial counts plated on L agar for colony forming units per ml (CFU/ml). This is done to ensure that low sedimental shear and control sedimental shear-grown Salmonella are in the same phase of growth for use in subsequent experiments.

Modulations in low shear sedimental culture conditions. Bacterial strains can be grown under the identical conditions above with the exception that the manipulations of the low sedimental shear environments are made within physiological ranges encountered by pathogens in the mammalian host. This can be done by the inclusion of inert beads of different sizes in the RWV bioreactor during cell culture, but other approaches are also possible.

Oral immunization of mice with attenuated Salmonella vaccine strains and protection against challenge with a virulent wild-type strain. Protective immunity elicited by attenuated Salmonella strains cultured under low shear sedimental and control shear sedimental conditions will determined in BALB/c mice following peroral (p.o.) inoculation. Six-to-ten-week-old female BALB/c mice (Charles River Laboratories, Wilmington, Mass.) will be immunized by peroral (p.o.) administration of serial dilutions of a low sedimental shear or control sedimental shear grown attenuated Salmonella vaccine strain. While this example focuses on oral infection of mice, other immunization methods can also be used, including peroral, intraperitoneal, nasal, vaginal administration, among others. Likewise, other hosts can be used for infection, including but not limited to, other animals, animal analogues, plants, insects, nematodes, and cell and tissue cultures from animals, animal analogues and plants. In addition, infections can be administered while both the host and pathogen are simultaneously in a low shear sedimental environment, including spaceflight. Mice are housed in autoclavable micro-isolator cages with free access to standard laboratory food and water for one week before use to allow acclimation. Bacteria for use in these studies are grown in the RWV under the conditions described above, harvested from the bioreactor by dispensing into a 50 ml polypropylene conical tube, and immediately harvested by centrifugation at room temperature for 10 minutes at 7,974×g. Bacteria are immediately resuspended in 1.0 ml buffered saline with gelatin (BSG).

Specifically, mice to be used in p.o. immunization with attenuated live vaccine strains or inoculation with challenge strains are deprived of food and water for 4-6 h. An attenuated Salmonella vaccine strain is grown simultaneously in the RWV bioreactors in the low shear sedimental conditions and control shear sedimental conditions and harvested as described above. Appropriate dilutions of the bacteria (low shear sedimental or control shear sedimental) will be prepared for p.o. inoculation of mice. Results will be obtained from ten mice/inoculum dose. Specifically, ten mice per group will be perorally inoculated with 10⁶, 10⁷, 10⁸, and 10⁹ CFU of the attenuated Salmonella vaccine strain grown under low shear sedimental or control shear sedimental conditions, respectively. Challenge with fully virulent wild-type Salmonella is given orally 30 days after immunization and mice are observed for four weeks thereafter. (Other routes of challenge may also be used). (In the case of recombinant attenuated Salmonella vaccine strain encoding heterologous antigen against another pathogen, challenge will also be with the fully virulent pathogen for which Salmonella carries the heterologous antigen. For example, for the recombinant attenuated Salmonella anti-pneumococcal vaccine strain, challenge would be with fully virulent Streptococcus pneumoniae.) Following challenge, mice will be monitored for signs of disease at least twice daily. These include a hunched posture, scruffy coat, and unwillingness to open eyes or move around. Mortality of the mice will be observed for 30 days. The median lethal dose will be determined by the method of Reed and Muench.

Enumeration of bacteria in mouse tissues. The effect of low sedimental shear on the tissue distribution and persistence of Salmonella in mice will be assessed in vivo by peroral inoculation into six-to-ten-week-old female BALB/c mice. Bacteria are grown and harvested as described above. Quantitation of viable Salmonella in tissues and organs will be performed as described previously from two groups of five mice each in two independent trials. The mice will be euthanized by CO₂ asphyxiation at 3, 5, and 7 days postinfection for subsequent harvesting of tissues and enumeration of bacteria to determine colonization of Salmonella. Thereafter, to determine persistence of Salmonella in mice, tissues will be harvested from mice weekly through through 60 days. Fecal pellets will also be collected to monitor shedding of Salmonella throughout the entire duration of the study. The number of Salmonella present in the tissues will be determined by viable counting of serial dilutions of the homogenates on MacConkey agar (Difco, Detroit, Mich.) supplemented with lactose at 1% final concentration. Murine tissues that will be analyzed include Peyer's patches, intestinal epithelium (minus Peyer's patches), liver, spleen and mesenteric lymph nodes.

Measurement and duration of antibody responses by ELISA following infection of animals with live attenuated recombinant bacterial vaccine strains carrying heterologous antigens. Groups of eight mice each will be immunized orally with different doses of a live attenuated Salmonella recombinant vaccine strain carrying a foreign antigen of interest and grown and harvested as described above. The live attenuated recombinant S. typhimurium vaccine strain used for the teaching the claims in this application express the pneumococcal PspA capsular antigen, however, any antigen(s) from any pathogen of interest could be used in these studies. Animal immunizations will be carried out perorally as described above. Booster immunizations may be given to enhance antibody responses to the foreign antigen. Serum samples (retroorbital puncture) and vaginal washings will be collected 2, 4, 6, and 8 weeks after immunization as described previously. Humoral, mucosal and cellular immune responses can be measured against Salmonella and/or to the heterologous antigen that it encodes.

The levels of antibodies present in mouse sera against the pneumococcal PspA capsular antigen and S. typhimurium LPS will be determined using enzyme-linked immunosorbent assay (ELISA) as follows. Ninety-six well Immulon plates (Dynatech, Chantilly, Va.) will be coated with 10 μg of a recombinant pneumococcal PspA capsular surface protein (rPspA) in 0.2 M bicarbonate/carbonate buffer (pH 9.6) at 4° C. overnight. Nonspecific binding sites will be blocked with 1% BSA in phosphate buffered saline (PBS)+0.1% Tween20 (pH 7.4) (blocking buffer) at room temperature for 1 h. Serum samples and vaginal washings will be diluted 1:100 and 1:10, respectively, in blocking buffer. One hundred microliters of the diluted samples will be added in duplicate to the plates and incubated at 37° C. for 2 h. The plates are then washed with PBS+0.1% Tween20 three times. One hundred microliters of biotin-labeled goat anti-mouse IgA or IgG will be added, respectively, and incubated at 4° C. overnight. Alkaline phosphatase-labeled ExtrAvidin (Sigma, St. Louis, Mo.) is added to the plates and incubated at room temperature for 1 h. Substrate solution (0.1 ml) containing p-nitro-phenylphosphate (1 mg/ml) in 0.1 M diethanolamine buffer (pH 9.8) will be added and the optical density of the resulting substrate reaction is read at 405 nm with an automated ELISA reader (BioTech, Burlington, Vt.).

Measurement of central memory T cells following infection of animals with live attenuated recombinant bacterial vaccine strains carrying heterologous antigens. The induction of memory responses is critical for the long-term protective efficacy of vaccines. In particular, the CD4+ CD44_{high} CD62L_{high} and CD8315 +CD44_{high}CD62L_{high} central memory T cells play a central role in the recall response (Krishnan, L., K. Gurnani, C. J. Dicaire, H. van Faassen, A. Zafer, C. J. Kirschning, S. Sad, and G. D. Sprott. 2007. Rapid clonal expansion and prolonged maintenance of memory CD8+ T Cells of the effector (CD44_{high}CD62L_{low}) and central (CD44_{high}CD62L_{high} phenotype by an archaeosome adjuvant independent of TLR2. J. Immunol. 178:2396-2406). Thus, the effect of low sedimental shear cultivation of the vaccine strain on stimulation of a memory T cell response will be evaluated. Eight weeks after immunization, spleens will be isolated from mice and compared to control (unimmunized and mock infected) mice. Splenic cells will be stimulated with antigen (rPspA) and then examined by FACS analysis for T cell markers indicative of memory cells.

Measurement of innate immune responses/cytokines following infection of animals with live attenuated recombinant bacterial vaccine strains carrying heterologous antigens. Six weeks after immunization, sera from immunized and control mice will be subjected to Bio-Plex Protein Array System (Bio-Rad, Hercules, Calif.) or ELISPOT analysis to determine antigen stimulation of cytokine production as described previously (Li Y, Wang S, Xin W, Scarpellini G, Shi Z, Gunn B, Roland K L, Curtiss R III. A sopB Deletion Mutation Enhances the Immunogenicity and Protective Efficacy of a Heterologous Antigen Delivered by Live Attenuated Salmonella enterica Vaccines. Infect Immun. 2008 Sep. 2. Epub ahead of print). The cytokine secretion profiles from splenic lymphocytes will be compared (other tissues may also be utilized). Both Th1 and Th2 cytokines will be profiled. Briefly, samples will be incubated with antibody-coupled beads for 1 h with shaking. Beads will be washed 3× with wash buffer to remove unbound protein and subsequently incubated with biotinylated detection cytokine-specific antibody for 1 h with shaking. The beads will then be washed once more followed by incubation for 10 min with streptavidin-phycoerythrin. After this incubation, beads will be washed and resuspended in assay buffer, and the contents of each well will be subjected to the flow-based Bio-Plex Suspension Array System, which identifies each different color bead as a population of protein and quantifies each protein target based on secondary antibody fluorescence. Cytokine concentrations will be calculated by Bio-Plex Manager software using a standard curve derived from a recombinant cytokine standard.

Immunoblotting for detection and quantiation of heterologous antigens carried by attenuated Salmonella vaccine strains from serum of infected animals. For immunoblotting, the S. typhimurium recombinant attenuated strain x9558 that carries the pneumococcal capsular antigen on plasmid pYA4088 will be grown with aeration overnight at 37° C. Five hundred microliters of each culture will be pelleted and resuspended in 2× sample loading buffer and boiled for 5 min. Protein preparations will be separated by sodium dodecyl sulfate (SDS)-polyacrylamide gel electrophoresis (PAGE) (12.5% polyacrylamide) and prepared for Coomassie brilliant blue staining or Western blot analysis. In addition to heterologous antigen detection, detection of Salmonella antigens can also be performed using Salmonella outer membrane protein antigens or LPS.

### Example 4

This example describes the stress response phenotypes observed for a recombinant attenuated Salmonella anti-pneumococcal vaccine strain, X9558 pYA4088 (Δpmi-2426 Δ(gmd-fcl)-26 ΔPfur₃₃::TTaraCP_{BAD}fur ΔPcrp₅₂₇::TTaraCP_{BAD}crp ΔasdA27::TTaraCP_{BADc2} ΔaraE25 ΔaraBAD23 ΔrelA198::araCP_{BAD}lacITT ΔsopB1925 ΔagfBAC811 ΔfliC180 ΔfljB217) during low fluid shear culture in the RWV bioreactor. The results indicate that low fluid shear culture in the RWV confers increased protection to the strain to survive virulence-related stress responses, including an increased ability to survive acid stress, thermal stress, and oxidative stress. In addition, low fluid shear culture of this strain decreased its biofilm formation. These low fluid shear-conferred phenotypes are all important features that could significantly enhance the immunogenicity and protection of this vaccine strain.

### Biofilm Formation

Differences in the O.D. readings before and after the RWVs were stopped:

| | O.D. before | O.D. after stopping | Duration of |
|---|---|---|---|
| | stopping HARV | HARV | experiment |
| 1XG | 0.495 | 0.923 | 25 hours 25 min |
| LSMMG | 1.376 | 1.462 | |

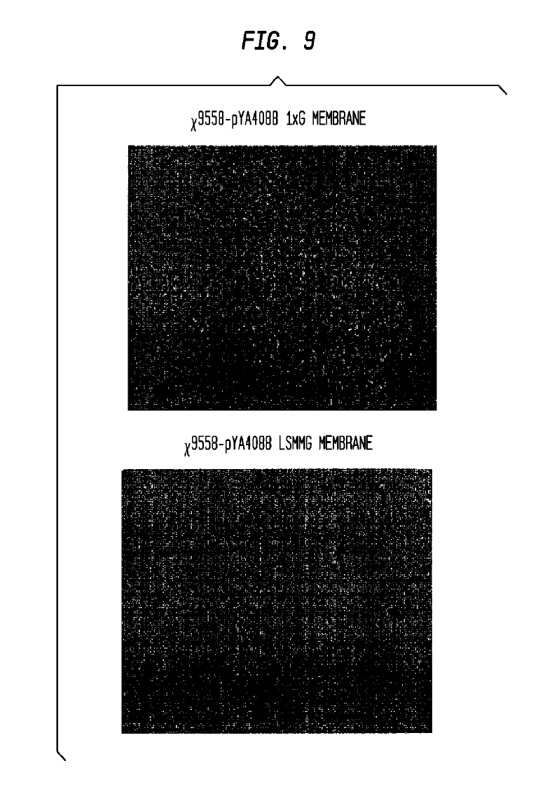

There is a marked difference in the O.D. reading for the 1×G condition before and after the RWV was stopped. This difference in OD was due to the enhanced biofilm formation in the 1×g condition as compared to the LSMMG condition, and not differences in cell numbers—as the cells were in the same phase of growth under the two conditions, however, most of the cells were sessile (attached to the membrane) in the 1×g RWV, leaving fewer cells in the supernatant (planktonic cells) for cell counting. When the RWVs were taken apart, a biofilm like substance was observed on the membrane with the 1×G culture. No such biofilm formation was observed for the LSMMG cultures. FIG. 9 presents microscopic images of cells scraped off of the membranes and stained with crystal violet.

### Thermal Stress Assay Data:

| | Time (min) | | |
|---|---|---|---|
| | 30 | 60 | 90 |
| LSMMG (37° C.) | 3.10E+08 | 2.67E+08 | 3.18E+08 |
| 1XG (37° C.) | 3.00E+08 | 3.35E+08 | 2.70E+08 |
| LSMMG (55° C.) | 3.80E+06 | 2.70E+05 | 0.00E+00 |
| 1XG (55° C.) | 7.60E+05 | 8.30E+04 | 0.00E+00 |

The data show that the cells can withstand thermal stress in the LSMMG condition much better as compared to the 1×G condition at 55° C.

### Acid Stress Assay Data:

In this experiment, LSMMG and 1×g cultures were subjected to acid stress and assayed for resistance to pH 3.5 (by addition of an amount of concentrated citrate buffer that has been previously determined to give this pH value) as described previously (Nickerson et al., Infection and Immunity, 2000; Wilson et al, Applied and Environmental Microbiology, 2002). The pH level during the assay was monitored using pH strips, and then confirmed with a pH electrode at the end of the assay. The percentage of surviving bacteria present after 30, 45, 60 and 90 minutes of acid stress (compared to the original number of bacteria before addition of the stress) was calculated via serial dilution and CFU plating. These results were compared identical control cultures that were not subjected to acid stress (no citrate buffer was added), but instead were allowed to sit on the bench top statically and at time points 30, 60 and 90 minutes about 100 ul were taken out and added to 9.9 ml of PBS. It can be seen that the LSMMG cells survive acid stress better than 1×G grown cells

| | Time (min) | | | | |
|---|---|---|---|---|---|
| | 0 | 30 | 45 | 60 | 90 |
| 1XG | 2.72E+06 | 2.66E+06 | | 2.72E+06 | 3.35E+06 |
| (Contro [symbol] | | | | | |
| LSMMG | 3.80E+06 | 3.37E+06 | | 3.66E+06 | 3.66E+06 |
| (Co [symbol] | | | | | |
| 1XG | 2.72E+06 | 2.07E+06 | 2.18E+06 | 2.27E+06 | 2.60E+06 |
| LSMMG | 3.80E+06 | 2.90E+06 | 3.26E+06 | 3.47E+06 | 2.82E+06 |

[symbol]

indicates data missing or illegible when filed

### Oxidative Stress Data:

In a separate experiment, oxidative stress (in the form of hydrogen peroxide) was applied to cells.

| | Time (min) | | | | |
|---|---|---|---|---|---|
| | 0 | 30 | 60 | 90 | 120 |
| 1XG | 6.13E+06 | 6.67E+06 | 6.72E+06 | 5.89E+06 | 7.10E+06 |
| (Contro [symbol] | | | | | |
| LSMMG | 6.30E+06 | 6.65E+06 | 6.19E+06 | 5.67E+06 | 6.49E+06 |
| (Co [symbol] | | | | | |
| 1XG | 6.13E+06 | 2.29E+04 | 0.00E+00 | 0.00E+00 | 0.00E+00 |
| LSMMG | 6.30E+06 | 1.43E+06 | 3.90E+05 | 2.10E+05 | 2.10E+05 |

[symbol]

indicates data missing or illegible when filed

It can be seen that the LSMMG cells survive the oxidative stress much better than the 1×G grown cells after 30 min.

The above results were obtained using the recombinant attenuated Salmonella anti-pneumococcal vaccine strain X9558 that carries the pneumococcal capsular antigen on plasmid pYA4088. The S. typhimurium UK-1 wild-type parent strain showed similar results.

### Example 5

### Spaceflight Alters Expression of Genes in the Hfq Regulon in Pseudomonas aeruginosa

Cultures of P. aeruginosa flew in the same flight experiment with S. typhimurium aboard STS-115 to determine changes in gene expression compared to otherwise identical ground controls. Preliminary results from microarray analysis indicate that of the 226 P. aeruginosa genes that were differentially regulated during spaceflight, 59 (˜23%) are regulated by Hfq—including those encoding ribosomal proteins, iron metabolic pathways, carbon metabolic pathways, cytoplasmic and periplasmic sigma factors, and ion response pathways. See Tables 7 and 8. In addition, it has been shown that LSMMG-cultured P. aeruginosa demonstrated an increased sensitivity to acid stress as compared to the control orientation. Collectively, these data supports an association between the gene expression and phenotypic response of P. aeruginosa during flight and LSMMG culture and the Hfq regulon.

### Example 6

### Spaceflight May Alter the Virulence Potential of Candida albicans

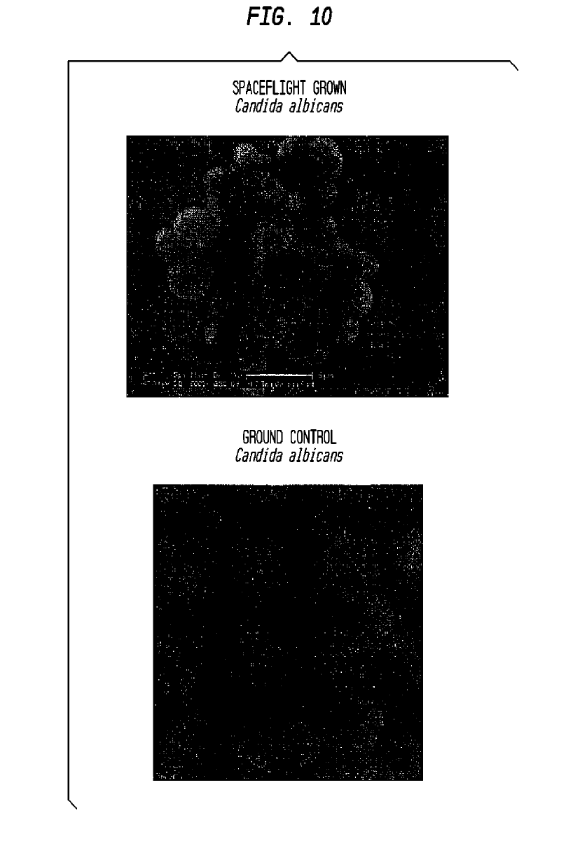

Scanning electron microscopy (SEM) (FIG. 10) shows profound hyphal formation of C. albicans during spaceflight culture—but no hyphal formation is evident during ground culture of identical controls. Hyphal formation is known to be associated with increased virulence.

### Example 7

### Phosphate Ion Modulates the LSMMG Response of the Gram Positive Pathogen, Staphylococcus aureus

Initial studies were performed on S. aureus N315 to determine phenotypic responses to culture in LSMMG and 1×G. When grown in LSMMG, S. aureus displayed a distinct decrease in the golden carotenoid pigmentation compared to growth in the control orientation, based upon a colorimetric assay for the primary carotenoid, staphyloxanthin. Notably, the addition of phosphate ion (25 mM Na₂HPO₄₎ to the media increased both the pigmentation of the LSSMG and 1×G control cultures. This latter finding is in agreement with the finding described above that environmental ions modulate the LSMMG acid stress response in Salmonella.

TABLE 7

| | | sig- | sig- | sig- | sig- | | | | |
|---|---|---|---|---|---|---|---|---|---|
| | | T- | T- | Mann- | Mann- | | | | |
| | % P | Test | Test | Whitney | Whitney | | sig log | sig log | sig log |
| | ground- | P | Change | P | Change | % MD/D- | ratio- | ratio- | ratio- |
| | Percent | Value | Direction | Value | Direction | Percent | Average | Median | Stdev |
| PA4243_secY_at (SEQ ID No: 639) | 100 | 0.262 | None | 0.05 | Down | 100 | −4.3 | −4.56 | 1.38 |
| PA4242_rpmJ_at (SEQ ID No: 640) | 100 | 0.206 | None | 0.05 | Down | 100 | −4.63 | −4.55 | 1.23 |
| PA5049_rpmE_at (SEQ ID No: 641) | 100 | 0.304 | None | 0.05 | Down | 88 | −4.14 | −4.54 | 1.39 |
| PA3745_rpsP_at (SEQ ID No: 642) | 100 | 0.275 | None | 0.05 | Down | 100 | −4.43 | −4.52 | 1.32 |
| PA3744_rimM_at (SEQ ID No: 643) | 100 | 0.261 | None | 0.05 | Down | 100 | −4.27 | −4.5 | 1.32 |
| PA4568_rplU_at (SEQ ID No: 644) | 100 | 0.256 | None | 0.05 | Down | 100 | −4.55 | −4.47 | 1.25 |
| PA2966_acpP_at (SEQ ID No: 645) | 100 | 0.213 | None | 0.05 | Down | 100 | −4.21 | −4.46 | 0.93 |
| PA0492_at (SEQ ID No: 646) | 100 | 0.045 | Down | 0.05 | Down | 100 | −4.29 | −4.43 | 0.78 |
| PA4563_rpsT_at (SEQ ID No: 647) | 100 | 0.186 | None | 0.05 | Down | 100 | −4.67 | −4.43 | 1.31 |
| PA4245_rpmD_at (SEQ ID No: 648) | 100 | 0.241 | None | 0.05 | Down | 100 | −4.12 | −4.42 | 1.16 |
| PA3656_rpsB_at (SEQ ID No: 649) | 100 | 0.233 | None | 0.05 | Down | 100 | −4.09 | −4.4 | 1.14 |
| PA2971_at (SEQ ID No: 650) | 100 | 0.331 | None | 0.05 | Down | 66 | −4.03 | −4.39 | 1.3 |
| PA4433_rplM_at (SEQ ID No: 651) | 100 | 0.228 | None | 0.05 | Down | 100 | −4.62 | −4.38 | 1.16 |
| PA5570_rpmH_at (SEQ ID No: 652) | 100 | 0.234 | None | 0.05 | Down | 66 | −4.58 | −4.37 | 1.03 |
| PA4240_rpsK_at (SEQ ID No: 653) | 100 | 0.26 | None | 0.05 | Down | 77 | −4.07 | −4.37 | 1.1 |
| PA4239_rpsD_at (SEQ ID No: 654) | 100 | 0.201 | None | 0.05 | Down | 100 | −4.29 | −4.37 | 0.99 |
| PA4262_rplD_at (SEQ ID No: 655) | 100 | 0.278 | None | 0.05 | Down | 100 | −4.04 | −4.25 | 1.1 |
| PA4268_rpsL_at (SEQ ID No: 656) | 100 | 0.278 | None | 0.05 | Down | 100 | −4.38 | −4.24 | 1.27 |
| PA4247_rplR_at (SEQ ID No: 657) | 100 | 0.232 | None | 0.05 | Down | 100 | −4.14 | −4.24 | 1 |
| PA4263_rplC_at (SEQ ID No: 658) | 100 | 0.261 | None | 0.05 | Down | 100 | −4.02 | −4.22 | 1.18 |
| PA3162_rpsA_at (SEQ ID No: 659) | 100 | 0.257 | None | 0.05 | Down | 77 | −3.94 | −4.17 | 0.99 |
| PA5316_rpmB_at (SEQ ID No: 660) | 100 | 0.271 | None | 0.05 | Down | 100 | −4.3 | −4.17 | 1.21 |
| Pae_tRNA_Gln_s_at (SEQ ID No: 661) | 100 | 0.331 | None | 0.05 | Down | 88 | −4.25 | −4.14 | 1.56 |
| PA4671_at (SEQ ID No: 662) | 100 | 0.196 | None | 0.05 | Down | 88 | −3.88 | −4.14 | 0.94 |
| PA4272_rplJ_at (SEQ ID No: 663) | 100 | 0.256 | None | 0.05 | Down | 88 | −3.84 | −4.14 | 0.97 |
| PA2619_infA_at (SEQ ID No: 664) | 100 | 0.175 | None | 0.05 | Down | 100 | −4.31 | −4.13 | 1.17 |
| PA4241_rpsM_at (SEQ ID No: 665) | 100 | 0.237 | None | 0.05 | Down | 88 | −3.87 | −4.12 | 1.11 |
| PA4482_gatC_at (SEQ ID No: 666) | 100 | 0.285 | None | 0.05 | Down | 77 | −4.01 | −4.11 | 0.84 |
| PA2743_infC_at (SEQ ID No: 667) | 100 | 0.326 | None | 0.05 | Down | 66 | −3.92 | −4.1 | 1.1 |
| PA3742_rplS_at (SEQ ID No: 668) | 100 | 0.263 | None | 0.05 | Down | 66 | −3.74 | −4.1 | 0.89 |
| PA5491_at (SEQ ID No: 669) | 100 | 0.248 | None | 0.05 | Down | 88 | −3.97 | −4.1 | 1.21 |
| PA4238_rpoA_at (SEQ ID No: 670) | 100 | 0.258 | None | 0.05 | Down | 88 | −3.74 | −4.1 | 0.99 |
| PA2321_at (SEQ ID No: 671) | 100 | 0.165 | None | 0.05 | Down | 100 | −4.16 | −4.1 | 1.04 |
| PA5276_lppL_i_at (SEQ ID No: 672) | 100 | 0.12 | None | 0.05 | Down | 55 | −4.05 | −4.09 | 0.98 |
| PA3743_trmD_at (SEQ ID No: 673) | 100 | 0.301 | None | 0.05 | Down | 88 | −4.19 | −4.09 | 1.39 |
| PA2639_nuoD_at (SEQ ID No: 674) | 100 | 0.045 | Down | 0.05 | Down | 100 | −3.9 | −4.09 | 0.68 |
| PA1800_tig_at (SEQ ID No: 675) | 100 | 0.34 | None | 0.05 | Down | 100 | −3.79 | −4.06 | 1.05 |
| PA5555_atpG_at (SEQ ID No: 676) | 100 | 0.246 | None | 0.05 | Down | 66 | −3.82 | −4.04 | 1.02 |
| PA2970_rpmF_at (SEQ ID No: 677) | 100 | 0.258 | None | 0.05 | Down | 100 | −4.1 | −4.04 | 1.19 |
| PA1582_sdhD_at (SEQ ID No: 678) | 100 | 0.145 | None | 0.05 | Down | 100 | −3.83 | −4.04 | 1.01 |
| PA4246_rpsE_at (SEQ ID No: 679) | 100 | 0.185 | None | 0.05 | Down | 66 | −3.73 | −4.01 | 1.06 |
| PA0579_rpsU_at (SEQ ID No: 680) | 100 | 0.273 | None | 0.05 | Down | 77 | −4.25 | −4.01 | 1.23 |
| PA2634_at (SEQ ID No: 681) | 100 | 0.008 | Down | 0.05 | Down | 100 | −3.84 | −3.99 | 0.66 |
| PA1581_sdhC_at (SEQ ID No: 682) | 100 | 0.264 | None | 0.05 | Down | 88 | −3.91 | −3.98 | 0.97 |
| PA5557_atpH_at (SEQ ID No: 683) | 100 | 0.318 | None | 0.05 | Down | 55 | −3.67 | −3.97 | 0.97 |
| Pae_tRNA_Gly_s_at (SEQ ID No: 684) | 100 | 0.32 | None | 0.05 | Down | 66 | −4.48 | −3.97 | 1.63 |
| PA5298_at (SEQ ID No: 685) | 100 | 0.072 | None | 0.05 | Down | 77 | −3.74 | −3.97 | 1.06 |
| PA4846_aroQ1_at (SEQ ID No: 686) | 100 | 0.153 | None | 0.05 | Down | 88 | −4.04 | −3.93 | 0.87 |
| PA0493_at (SEQ ID No: 687) | 100 | 0.007 | Down | 0.05 | Down | 100 | −4.05 | −3.93 | 0.87 |
| PA3266_capB_at (SEQ ID No: 688) | 100 | 0.178 | None | 0.05 | Down | 66 | −4.01 | −3.91 | 1.16 |
| PA4267_rpsG_at (SEQ ID No: 689) | 100 | 0.229 | None | 0.05 | Down | 55 | −3.82 | −3.9 | 0.97 |
| PA4748_tpiA_at (SEQ ID No: 690) | 100 | 0.339 | None | 0.05 | Down | 55 | −3.72 | −3.9 | 1.26 |
| PA4847_accB_at (SEQ ID No: 691) | 100 | 0.165 | None | 0.05 | Down | 77 | −3.77 | −3.9 | 0.74 |
| Pae_tRNA_Val_f_at (SEQ ID No: 692) | 100 | 0.323 | None | 0.05 | Down | 66 | −3.8 | −3.88 | 1.25 |
| PA4249_rpsH_at (SEQ ID No: 693) | 100 | 0.286 | None | 0.05 | Down | 77 | −3.83 | −3.84 | 1.25 |
| PA0856_at (SEQ ID No: 694) | 100 | 0.041 | Down | 0.05 | Down | 100 | −3.71 | −3.84 | 0.77 |
| PA5569_rnpA_at (SEQ ID No: 695) | 100 | 0.282 | None | 0.05 | Down | 55 | −3.82 | −3.83 | 1.18 |
| PA0896_aruF_at (SEQ ID No: 696) | 100 | 0.047 | Down | 0.05 | Down | 77 | −3.99 | −3.83 | 1.32 |
| PA4430_at (SEQ ID No: 697) | 100 | 0.142 | None | 0.05 | Down | 77 | −3.9 | −3.81 | 0.86 |
| PA4935_rpsF_at (SEQ ID No: 698) | 100 | 0.305 | None | 0.05 | Down | 55 | −3.57 | −3.8 | 0.92 |
| PA4031_ppa_at (SEQ ID No: 699) | 100 | 0.146 | None | 0.05 | Down | 66 | −3.54 | −3.78 | 0.76 |
| PA2744_thrS_at (SEQ ID No: 700) | 100 | 0.239 | None | 0.05 | Down | 55 | −3.48 | −3.76 | 0.96 |
| PA4942_hflK_at (SEQ ID No: 701) | 100 | 0.245 | None | 0.05 | Down | 55 | −3.57 | −3.75 | 0.99 |
| PA1557_at (SEQ ID No: 702) | 100 | 0.073 | None | 0.05 | Down | 55 | −3.87 | −3.74 | 0.75 |
| PA4406_lpxC_at (SEQ ID No: 703) | 100 | 0.215 | None | 0.05 | Down | 66 | −3.62 | −3.74 | 0.95 |
| PA3621_fdxA_at (SEQ ID No: 704) | 100 | 0.157 | None | 0.05 | Down | 88 | −3.76 | −3.73 | 0.98 |
| PA3644_lpxA_at (SEQ ID No: 705) | 100 | 0.238 | None | 0.05 | Down | 88 | −3.63 | −3.73 | 1.06 |
| PA4053_ribE_at (SEQ ID No: 706) | 100 | 0.233 | None | 0.05 | Down | 88 | −3.57 | −3.73 | 0.83 |
| PA4276_secE_at (SEQ ID No: 707) | 100 | 0.334 | None | 0.05 | Down | 55 | −3.87 | −3.69 | 1.36 |
| PA3811_hscB_at (SEQ ID No: 708) | 100 | 0.281 | None | 0.05 | Down | 55 | −3.45 | −3.69 | 0.93 |
| PA3645_fabZ_at (SEQ ID No: 709) | 100 | 0.207 | None | 0.05 | Down | 66 | −3.6 | −3.69 | 0.99 |
| PA4431_at (SEQ ID No: 710) | 100 | 0.201 | None | 0.05 | Down | 77 | −3.51 | −3.69 | 0.8 |
| PA1156_nrdA_at (SEQ ID No: 711) | 100 | 0.137 | None | 0.05 | Down | 88 | −3.74 | −3.69 | 0.83 |
| PA4762_grpE_at (SEQ ID No: 712) | 100 | 0.289 | None | 0.05 | Down | 88 | −3.64 | −3.68 | 1.02 |
| PA3159_wbpA_at (SEQ ID No: 713) | 100 | 0.339 | None | 0.05 | Down | 55 | −3.49 | −3.67 | 0.88 |
| PA4944_at (SEQ ID No: 714) | 100 | 0.304 | None | 0.05 | Down | 66 | −3.54 | −3.66 | 0.97 |
| PA3636_kdsA_at (SEQ ID No: 715) | 100 | 0.125 | None | 0.05 | Down | 66 | −3.52 | −3.66 | 0.87 |
| PA4261_rplW_at (SEQ ID No: 716) | 100 | 0.29 | None | 0.05 | Down | 77 | −3.8 | −3.65 | 1.14 |
| PA4252_rplX_at (SEQ ID No: 717) | 100 | 0.003 | Down | 0.05 | Down | 100 | −3.95 | −3.65 | 1.08 |
| PA4745_nusA_at (SEQ ID No: 718) | 100 | 0.301 | None | 0.05 | Down | 55 | −3.38 | −3.62 | 0.97 |
| PA3635_eno_at (SEQ ID No: 719) | 100 | 0.269 | None | 0.05 | Down | 55 | −3.28 | −3.62 | 0.9 |
| PA0336_at (SEQ ID No: 720) | 100 | 0.289 | None | 0.05 | Down | 77 | −3.43 | −3.62 | 0.85 |
| ig_5207621_5208463_at | 100 | 0.202 | None | 0.05 | Down | 55 | −3.5 | −3.61 | 0.95 |
| (SEQ ID No: 721) | | | | | | | | | |
| PA5558_atpF_at (SEQ ID No: 722) | 100 | 0.304 | None | 0.05 | Down | 77 | −3.51 | −3.61 | 0.96 |
| PA2968_fabD_at (SEQ ID No: 723) | 100 | 0.202 | None | 0.05 | Down | 66 | −3.6 | −3.6 | 0.93 |
| PA3832_holC_at (SEQ ID No: 724) | 100 | 0.017 | Down | 0.05 | Down | 88 | −3.55 | −3.59 | 0.66 |
| PA4483_gatA_at (SEQ ID No: 725) | 100 | 0.153 | None | 0.05 | Down | 66 | −3.62 | −3.58 | 0.76 |
| PA2747_at (SEQ ID No: 726) | 100 | 0.085 | None | 0.05 | Down | 66 | −3.76 | −3.57 | 0.82 |
| Pae_tRNA_His_f_at (SEQ ID No: 727) | 100 | 0.184 | None | 0.05 | Down | 66 | −3.66 | −3.57 | 1.04 |
| PA2624_idh_at (SEQ ID No: 728) | 100 | 0.177 | None | 0.05 | Down | 77 | −3.32 | −3.57 | 0.67 |
| PA2453_at (SEQ ID No: 729) | 100 | 0.23 | None | 0.05 | Down | 88 | −3.52 | −3.56 | 0.82 |
| PA4258_rplV_at (SEQ ID No: 730) | 100 | 0.3 | None | 0.05 | Down | 55 | −3.57 | −3.55 | 0.99 |
| PA4743_rbfA_at (SEQ ID No: 731) | 100 | 0.229 | None | 0.05 | Down | 55 | −3.52 | −3.55 | 1.06 |
| PA1533_at (SEQ ID No: 732) | 100 | 0.147 | None | 0.05 | Down | 77 | −3.69 | −3.54 | 0.94 |
| PA1123_at (SEQ ID No: 733) | 100 | 0.004 | Down | 0.05 | Down | 88 | −3.49 | −3.54 | 0.72 |
| PA5490_cc4_at (SEQ ID No: 734) | 100 | 0.114 | None | 0.05 | Down | 88 | −3.59 | −3.54 | 0.8 |
| PA3001_at (SEQ ID No: 735) | 100 | 0.078 | None | 0.05 | Down | 100 | −3.74 | −3.54 | 0.87 |
| PA1013_purC_at (SEQ ID No: 736) | 100 | 0.199 | None | 0.05 | Down | 88 | −3.35 | −3.53 | 0.6 |
| PA3987_leuS_at (SEQ ID No: 737) | 100 | 0.079 | None | 0.05 | Down | 55 | −3.59 | −3.52 | 1.14 |
| PA4761_dnaK_at (SEQ ID No: 738) | 100 | 0.152 | None | 0.05 | Down | 66 | −3.46 | −3.52 | 0.8 |
| PA3701_prfB_at (SEQ ID No: 739) | 100 | 0.117 | None | 0.05 | Down | 66 | −3.42 | −3.51 | 0.72 |
| PA5128_secB_at (SEQ ID No: 740) | 100 | 0.054 | None | 0.05 | Down | 100 | −3.66 | −3.51 | 0.71 |
| PA4253_rplN_at (SEQ ID No: 741) | 100 | 0.255 | None | 0.05 | Down | 66 | −3.57 | −3.5 | 0.97 |
| PA4386_groES_at (SEQ ID No: 742) | 100 | 0.228 | None | 0.05 | Down | 77 | −3.65 | −3.5 | 0.94 |
| PA5067_hisE_at (SEQ ID No: 743) | 100 | 0.148 | None | 0.05 | Down | 77 | −3.31 | −3.5 | 0.77 |
| PA4880_at (SEQ ID No: 744) | 100 | 0.1 | None | 0.05 | Down | 88 | −3.49 | −3.5 | 0.94 |
| PA1610_fabA_at (SEQ ID No: 745) | 100 | 0.118 | None | 0.05 | Down | 100 | −3.77 | −3.5 | 1 |
| PA3807_ndk_at (SEQ ID No: 746) | 100 | 0.279 | None | 0.05 | Down | 66 | −3.56 | −3.49 | 0.95 |
| PA4266_fusA1_at (SEQ ID No: 747) | 100 | 0.284 | None | 0.05 | Down | 77 | −3.69 | −3.48 | 1.05 |
| PA0972_tolB_at (SEQ ID No: 748) | 100 | 0.198 | None | 0.05 | Down | 66 | −3.52 | −3.47 | 0.95 |
| PA4232_ssb_at (SEQ ID No: 749) | 100 | 0.114 | None | 0.05 | Down | 88 | −3.46 | −3.47 | 0.75 |
| PA3700_lysS_at (SEQ ID No: 750) | 100 | 0.251 | None | 0.05 | Down | 55 | −3.5 | −3.45 | 0.9 |
| PA4460_at (SEQ ID No: 751) | 100 | 0.147 | None | 0.05 | Down | 66 | −3.42 | −3.45 | 0.88 |
| PA5069_tatB_at (SEQ ID No: 752) | 100 | 0.233 | None | 0.05 | Down | 66 | −3.65 | −3.44 | 0.94 |
| PA4853_fis_at (SEQ ID No: 753) | 100 | 0.188 | None | 0.05 | Down | 66 | −3.37 | −3.44 | 0.9 |
| PA0019_def_at (SEQ ID No: 754) | 100 | 0.037 | Down | 0.05 | Down | 77 | −3.43 | −3.44 | 0.57 |
| PA0595_ostA_at (SEQ ID No: 755) | 100 | 0.182 | None | 0.05 | Down | 55 | −3.42 | −3.43 | 0.98 |
| PA4848_accC_at (SEQ ID No: 756) | 100 | 0.109 | None | 0.05 | Down | 77 | −3.57 | −3.43 | 0.88 |
| PA1580_gltA_at (SEQ ID No: 757) | 100 | 0.162 | None | 0.05 | Down | 77 | −3.38 | −3.43 | 0.8 |
| PA4259_rpsS_at (SEQ ID No: 758) | 100 | 0.152 | None | 0.05 | Down | 55 | −3.74 | −3.41 | 0.92 |
| PA2976_rne_at (SEQ ID No: 759) | 100 | 0.096 | None | 0.05 | Down | 66 | −3.44 | −3.41 | 0.74 |
| PA1574_at (SEQ ID No: 760) | 100 | 0.015 | Down | 0.05 | Down | 77 | −3.47 | −3.41 | 1.07 |
| PA2023_galU_at (SEQ ID No: 761) | 100 | 0.029 | Down | 0.05 | Down | 77 | −3.46 | −3.41 | 0.64 |
| PA4740_pnp_at (SEQ ID No: 762) | 100 | 0.194 | None | 0.05 | Down | 77 | −3.45 | −3.4 | 0.74 |
| PA3907_at (SEQ ID No: 763) | 100 | 0.158 | None | 0.05 | Down | 55 | −3.26 | −3.39 | 0.72 |
| PA2960_pilZ_at (SEQ ID No: 764) | 100 | 0.079 | None | 0.05 | Down | 66 | −3.48 | −3.38 | 0.84 |
| PA4425_at (SEQ ID No: 765) | 100 | 0.111 | None | 0.05 | Down | 55 | −3.46 | −3.37 | 0.82 |
| PA5068_tatA_at (SEQ ID No: 766) | 100 | 0.159 | None | 0.05 | Down | 55 | −3.37 | −3.37 | 0.76 |
| PA3686_adk_at (SEQ ID No: 767) | 100 | 0.177 | None | 0.05 | Down | 88 | −3.73 | −3.37 | 0.88 |
| PA4759_dapB_at (SEQ ID No: 768) | 100 | 0.043 | Down | 0.05 | Down | 77 | −3.38 | −3.36 | 0.83 |
| PA4292_at (SEQ ID No: 769) | 100 | 0.155 | None | 0.05 | Down | 77 | −3.41 | −3.36 | 0.94 |
| PA1552_at (SEQ ID No: 770) | 100 | 0.024 | Down | 0.05 | Down | 88 | −3.55 | −3.36 | 0.87 |
| PA5143_hisB_at (SEQ ID No: 771) | 100 | 0.03 | Down | 0.05 | Down | 66 | −3.4 | −3.35 | 0.78 |
| PA3014_faoA_at (SEQ ID No: 772) | 100 | 0.104 | None | 0.05 | Down | 66 | −3.34 | −3.35 | 0.73 |
| PA1505_moaA2_at (SEQ ID No: 773) | 100 | 0.082 | None | 0.05 | Down | 66 | −3.3 | −3.34 | 0.87 |
| PA3981_at (SEQ ID No: 774) | 100 | 0.038 | Down | 0.05 | Down | 77 | −3.51 | −3.34 | 0.91 |
| PA2965_fabF1_at (SEQ ID No: 775) | 100 | 0.265 | None | 0.05 | Down | 55 | −3.33 | −3.3 | 0.92 |
| PA0857_bolA_at (SEQ ID No: 776) | 100 | 0.103 | None | 0.05 | Down | 66 | −3.36 | −3.3 | 0.75 |
| PA5315_rpmG_at (SEQ ID No: 777) | 100 | 0.052 | None | 0.05 | Down | 77 | −3.47 | −3.3 | 0.87 |
| PA3861_rhlB_at (SEQ ID No: 778) | 100 | 0.059 | None | 0.05 | Down | 66 | −3.32 | −3.28 | 0.85 |
| Pae_tRNA_Asn_s_at (SEQ ID No: 779) | 100 | 0.081 | None | 0.05 | Down | 55 | −3.28 | −3.27 | 0.79 |
| PA3575_at (SEQ ID No: 780) | 100 | 0.092 | None | 0.05 | Down | 88 | −3.48 | −3.27 | 0.88 |
| PA1774_at (SEQ ID No: 781) | 100 | 0.167 | None | 0.05 | Down | 55 | −3.21 | −3.26 | 0.73 |
| PA4333_at (SEQ ID No: 782) | 100 | 0.248 | None | 0.05 | Down | 66 | −3.26 | −3.25 | 0.91 |
| PA2667_at (SEQ ID No: 783) | 100 | 0.115 | None | 0.05 | Down | 66 | −3.24 | −3.24 | 0.65 |
| PA2979_kdsB_at (SEQ ID No: 784) | 100 | 0.065 | None | 0.05 | Down | 55 | −3.36 | −3.23 | 0.87 |
| PA4006_at (SEQ ID No: 785) | 100 | 0.184 | None | 0.05 | Down | 66 | −3.48 | −3.23 | 1.07 |
| PA1008_bcp_at (SEQ ID No: 786) | 100 | 0.032 | Down | 0.05 | Down | 66 | −3.26 | −3.22 | 0.72 |
| PA4271_rplL_at (SEQ ID No: 787) | 100 | 0.271 | None | 0.05 | Down | 66 | −3.55 | −3.22 | 1.02 |
| PA3480_at (SEQ ID No: 788) | 100 | 0.124 | None | 0.05 | Down | 77 | −3.49 | −3.22 | 1.04 |
| PA4503_at (SEQ ID No: 789) | 100 | 0.178 | None | 0.05 | Down | 77 | −3.25 | −3.22 | 0.72 |
| PA5119_glnA_at (SEQ ID No: 790) | 100 | 0.071 | None | 0.05 | Down | 88 | −3.32 | −3.22 | 0.68 |
| PA5054_hslU_at (SEQ ID No: 791) | 100 | 0.088 | None | 0.05 | Down | 100 | −3.5 | −3.22 | 0.83 |
| PA2950_at (SEQ ID No: 792) | 100 | 0.159 | None | 0.05 | Down | 55 | −3.2 | −3.21 | 0.65 |
| PA1609_fabB_at (SEQ ID No: 793) | 100 | 0.054 | None | 0.05 | Down | 77 | −3.23 | −3.2 | 0.55 |
| PA3637_pyrG_at (SEQ ID No: 794) | 100 | 0.147 | None | 0.05 | Down | 88 | −3.52 | −3.2 | 0.86 |
| PA5429_aspA_at (SEQ ID No: 795) | 100 | 0.058 | None | 0.05 | Down | 77 | −3.44 | −3.19 | 0.72 |
| PA5322_algC_at (SEQ ID No: 796) | 100 | 0.084 | None | 0.05 | Down | 100 | −3.5 | −3.19 | 0.63 |
| PA0429_at (SEQ ID No: 797) | 100 | 0.148 | None | 0.05 | Down | 66 | −3.49 | −3.18 | 1.03 |
| PA3369_at (SEQ ID No: 798) | 100 | 0.079 | None | 0.05 | Down | 77 | −3.3 | −3.18 | 0.74 |
| PA4559_lspA_at (SEQ ID No: 799) | 100 | 0.279 | None | 0.05 | Down | 55 | −3.28 | −3.17 | 0.8 |
| PA1659_at (SEQ ID No: 800) | 100 | 0.064 | None | 0.05 | Down | 77 | −3.26 | −3.17 | 0.56 |
| PA0357_mutM_at (SEQ ID No: 801) | 100 | 0.078 | None | 0.05 | Down | 55 | −3.26 | −3.16 | 0.72 |
| PA5063_ubiE_at (SEQ ID No: 802) | 100 | 0.024 | Down | 0.05 | Down | 100 | −3.25 | −3.16 | 0.74 |
| PA0555_fda_at (SEQ ID No: 803) | 100 | 0.094 | None | 0.05 | Down | 77 | −3.21 | −3.13 | 0.72 |
| PA3440_at (SEQ ID No: 804) | 100 | 0.121 | None | 0.05 | Down | 88 | −3.38 | −3.13 | 0.91 |
| PA1009_at (SEQ ID No: 805) | 100 | 0.077 | None | 0.05 | Down | 55 | −3.14 | −3.12 | 0.76 |
| PA1010_dapA_at (SEQ ID No: 806) | 100 | 0.114 | None | 0.05 | Down | 66 | −3.41 | −3.12 | 0.7 |
| PA3626_at (SEQ ID No: 807) | 100 | 0.094 | None | 0.05 | Down | 55 | −3.16 | −3.1 | 0.66 |
| PA1462_at (SEQ ID No: 808) | 100 | 0.131 | None | 0.05 | Down | 77 | −3.23 | −3.1 | 0.75 |
| PA4264_rpsJ_at (SEQ ID No: 809) | 100 | 0.301 | None | 0.05 | Down | 55 | −3.69 | −3.09 | 1.31 |
| PA5078_at (SEQ ID No: 810) | 100 | 0.081 | None | 0.05 | Down | 100 | −3.34 | −3.09 | 0.78 |
| PA0766_mucD_at (SEQ ID No: 811) | 100 | 0.1 | None | 0.05 | Down | 66 | −3.14 | −3.08 | 0.64 |
| PA1183_dctA_at (SEQ ID No: 812) | 100 | 0.053 | None | 0.05 | Down | 77 | −3.39 | −3.08 | 0.88 |
| PA5000_at (SEQ ID No: 813) | 100 | 0.075 | None | 0.05 | Down | 55 | −3.29 | −3.07 | 1.1 |
| PA3770_guaB_at (SEQ ID No: 814) | 100 | 0.076 | None | 0.05 | Down | 66 | −3.32 | −3.07 | 0.76 |
| PA5323_argB_at (SEQ ID No: 815) | 100 | 0.071 | None | 0.05 | Down | 66 | −3.19 | −3.07 | 0.76 |
| PA5174_at (SEQ ID No: 816) | 100 | 0.029 | Down | 0.05 | Down | 55 | −3.18 | −3.06 | 0.91 |
| PA3171_ubiG_at (SEQ ID No: 817) | 100 | 0.034 | Down | 0.05 | Down | 55 | −3.25 | −3.05 | 0.65 |
| PA1482_ccmH_at (SEQ ID No: 818) | 100 | 0.099 | None | 0.05 | Down | 55 | −3.34 | −3.04 | 0.79 |
| PA2646_nuoK_at (SEQ ID No: 819) | 100 | 0.225 | None | 0.05 | Down | 55 | −3.25 | −3.04 | 0.9 |
| PA2612_serS_at (SEQ ID No: 820) | 100 | 0.228 | None | 0.05 | Down | 66 | −3.29 | −3.03 | 0.75 |
| PA0527_dnr_at (SEQ ID No: 821) | 100 | 0.164 | None | 0.05 | Down | 88 | −3.13 | −3.03 | 0.64 |
| PA1660_at (SEQ ID No: 822) | 100 | 0.024 | Down | 0.05 | Down | 77 | −3.4 | −3.02 | 0.67 |
| PA3962_at (SEQ ID No: 823) | 100 | 0.01 | Down | 0.05 | Down | 77 | −3.35 | −3.02 | 0.79 |
| PA3031_at (SEQ ID No: 824) | 100 | 0.073 | None | 0.05 | Down | 88 | −3.28 | −3.02 | 0.79 |
| PA2780_at (SEQ ID No: 825) | 100 | 0.07 | None | 0.05 | Down | 88 | −3.37 | −2.99 | 0.88 |
| PA2649_nuoN_at (SEQ ID No: 826) | 100 | 0.101 | None | 0.05 | Down | 55 | −3.25 | −2.98 | 0.75 |
| PA2644_nuoI_at (SEQ ID No: 827) | 100 | 0.092 | None | 0.05 | Down | 66 | −3.12 | −2.97 | 0.62 |
| PA0730_at (SEQ ID No: 828) | 100 | 0.128 | None | 0.05 | Down | 66 | −3.23 | −2.94 | 0.93 |
| PA4403_secA_at (SEQ ID No: 829) | 100 | 0.058 | None | 0.05 | Down | 66 | −3.13 | −2.93 | 0.79 |
| PA3476_rhlL_at (SEQ ID No: 830) | 100 | 0.029 | Down | 0.05 | Down | 100 | −3.29 | −2.93 | 0.73 |
| PA3286_at (SEQ ID No: 831) | 100 | 0.018 | Down | 0.05 | Down | 66 | −3.22 | −2.92 | 0.87 |
| PA2980_at (SEQ ID No: 832) | 100 | 0.058 | None | 0.05 | Down | 66 | −3.16 | −2.91 | 0.8 |
| PA1642_selD_at (SEQ ID No: 833) | 100 | 0.006 | Down | 0.05 | Down | 55 | −3.03 | −2.9 | 0.66 |
| PA0537_at (SEQ ID No: 834) | 100 | 0.061 | None | 0.05 | Down | 66 | −3.12 | −2.9 | 0.75 |
| PA3524_gloA1_at (SEQ ID No: 835) | 100 | 0.07 | None | 0.05 | Down | 77 | −3.2 | −2.9 | 0.79 |
| PA5134_at (SEQ ID No: 836) | 100 | 0.023 | Down | 0.05 | Down | 55 | −3.12 | −2.89 | 0.79 |
| PA5038_aroB_at (SEQ ID No: 837) | 100 | 0.044 | Down | 0.05 | Down | 66 | −3.18 | −2.89 | 0.7 |
| PA5076_at (SEQ ID No: 838) | 100 | 0.114 | None | 0.05 | Down | 66 | −3.19 | −2.89 | 0.78 |
| PA2528_at (SEQ ID No: 839) | 100 | 0.024 | Down | 0.05 | Down | 55 | −3.11 | −2.88 | 0.77 |
| PA1504_at (SEQ ID No: 840) | 100 | 0.146 | None | 0.05 | Down | 66 | −3.11 | −2.88 | 0.79 |
| PA1102_fliG_at (SEQ ID No: 841) | 100 | 0.136 | None | 0.05 | Down | 55 | −3.13 | −2.87 | 0.81 |
| PA1421_speB2_at (SEQ ID No: 842) | 100 | 0.089 | None | 0.05 | Down | 88 | −3.18 | −2.87 | 0.79 |
| PA0582_folB_at (SEQ ID No: 843) | 100 | 0.036 | Down | 0.05 | Down | 55 | −3.21 | −2.86 | 0.87 |
| PA4411_murC_at (SEQ ID No: 844) | 100 | 0.019 | Down | 0.05 | Down | 55 | −3.11 | −2.86 | 0.86 |
| PA4054_ribB_at (SEQ ID No: 845) | 100 | 0.031 | Down | 0.05 | Down | 55 | −3.05 | −2.84 | 0.59 |
| PA1677_at (SEQ ID No: 846) | 100 | 0.108 | None | 0.05 | Down | 55 | −3.07 | −2.84 | 0.73 |
| PA5224_pepP_at (SEQ ID No: 847) | 100 | 0.06 | None | 0.05 | Down | 55 | −3.06 | −2.84 | 0.62 |
| PA0943_at (SEQ ID No: 848) | 100 | 0.164 | None | 0.05 | Down | 55 | −3.01 | −2.82 | 0.78 |
| PA1420_at (SEQ ID No: 849) | 100 | 0.058 | None | 0.05 | Down | 66 | −3.15 | −2.81 | 0.69 |
| PA5064_at (SEQ ID No: 850) | 100 | 0.059 | None | 0.05 | Down | 66 | −3.13 | −2.81 | 0.86 |
| PA4423_at (SEQ ID No: 851) | 100 | 0.075 | None | 0.05 | Down | 55 | −3 | −2.79 | 0.72 |
| PA3566_at (SEQ ID No: 852) | 100 | 0.012 | Down | 0.05 | Down | 88 | −3.06 | −2.78 | 0.61 |
| PA0900_at (SEQ ID No: 853) | 100 | 0.055 | None | 0.05 | Down | 66 | −3.02 | −2.77 | 0.7 |
| PA2953_at (SEQ ID No: 854) | 100 | 0.072 | None | 0.05 | Down | 55 | −3.09 | −2.74 | 0.86 |
| PA5344_at (SEQ ID No: 855) | 100 | 0.07 | None | 0.05 | Down | 55 | −3.02 | −2.74 | 0.66 |
| PA4345_at (SEQ ID No: 856) | 100 | 0.097 | None | 0.05 | Down | 55 | −2.95 | −2.73 | 0.6 |
| PA3262_at (SEQ ID No: 857) | 100 | 0.06 | None | 0.05 | Down | 55 | −3.04 | −2.68 | 0.62 |
| PA5227_at (SEQ ID No: 858) | 100 | 0.193 | None | 0.05 | Down | 55 | −2.98 | −2.68 | 0.74 |
| PA0083_at (SEQ ID No: 859) | 100 | 0.023 | Down | 0.05 | Down | 55 | −3.07 | −2.65 | 0.79 |
| PA2379_at (SEQ ID No: 860) | 100 | 0.048 | Down | 0.05 | Down | 77 | −3.18 | −2.64 | 0.86 |
| PA5479_gltP_at (SEQ ID No: 861) | 100 | 0.139 | None | 0.05 | Down | 55 | −3.05 | −2.62 | 0.93 |
| PA2322_at (SEQ ID No: 862) | 100 | 0.168 | None | 0.05 | Down | 77 | −3.07 | −2.62 | 0.88 |

Description
PA4243 /GENE = secY /DEF = secretion protein SecY /FUNCTION = Membrane proteins; Protein secretion/export apparatus (SEQ ID No: 639)
PA4242/GENE = rpmJ /DEF = 50S ribosomal protein L36 /FUNCTION = Translation, post-translational modification, degradation (SEQ ID No: 640)
PA5049/GENE = rpmE /DEF = 50S ribosomal protein L31 /FUNCTION = Translation, post-translational modification, degradation (SEQ ID No: 641)
PA3745/GENE = rpsP /DEF = 30S ribosomal protein S16 /FUNCTION = Translation, post-translational modification, degradation; DNA replication, recombination, modification and repair (SEQ ID No: 642)
PA3744 /GENE = rimM /DEF = 16S rRNA processing protein /FUNCTION = Transcription, RNA processing and degradation (SEQ ID No: 643)
PA4568/GENE = rplU /DEF = 50S ribosomal protein L21 /FUNCTION = Translation, post-translational modification, degradation (SEQ ID No: 644)
PA2966/GENE = acpP /DEF = acyl carrier protein /FUNCTION = Fatty acid and phospholipid metabolism (SEQ ID No: 645)
PA0492/DEF = conserved hypothetical protein /FUNCTION = Hypothetical, unclassified, unknown (SEQ ID No: 646)
PA4563/GENE = rpsT /DEF = 30S ribosomal protein S20 /FUNCTION = Translation, post-translational modification, degradation; Central intermediary metabolism (SEQ ID No: 647)
PA4245/GENE = rpmD /DEF = 50S ribosomal protein L30 /FUNCTION = Translation, post-translational modification, degradation (SEQ ID No: 648)
PA3656/GENE = rpsB /DEF = 30S ribosomal protein S2 /FUNCTION = Translation, post-translational modification, degradation (SEQ ID No: 649)
PA2971/DEF = conserved hypothetical protein /FUNCTION = Hypothetical, unclassified, unknown (SEQ ID No: 650)
PA4433 /GENE = rplM /DEF = 50S ribosomal protein L13 /FUNCTION = Translation, post-translational modification, degradation (SEQ ID No: 651)
PA5570/GENE = rpmH /DEF = 50S ribosomal protein L34 /FUNCTION = Central intermediary metabolism; Translation, post-translational modification, degradation (SEQ ID No: 652)
PA4240/GENE = rpsK /DEF = 30S ribosomal protein S11 /FUNCTION = Translation, post-translational modification, degradation (SEQ ID No: 653)
PA4239/GENE = rpsD /DEF = 30S ribosomal protein S4 /FUNCTION = Translation, post-translational modification, degradation (SEQ ID No: 654)
PA4262/GENE = rplD /DEF = 50S ribosomal protein L4 /FUNCTION = Transcription, RNA processing and degradation; Translation, post-translational modification, degradation (SEQ ID No: 655)
PA4268 /GENE = rpsL /DEF = 30S ribosomal protein S12 /FUNCTION = Translation, post-translational modification, degradation (SEQ ID No: 656)
PA4247/GENE = rplR /DEF = 50S ribosomal protein L18 /FUNCTION = Translation, post-translational modification, degradation (SEQ ID No: 657)
PA4263/GENE = rplC /DEF = 50S ribosomal protein L3 /FUNCTION = Translation, post-translational modification, degradation (SEQ ID No: 658)
PA3162/GENE = rpsA /DEF = 30S ribosomal protein S1 /FUNCTION = Translation, post-translational modification, degradation (SEQ ID No: 659)
PA5316/GENE = rpmB /DEF = 50S ribosomal protein L28 /FUNCTION = Translation, post-translational modification, degradation (SEQ ID No: 660)
tRNA_Glutamine, 5238277-5238351 (+) strand (SEQ ID No: 661)
PA4671/DEF = probable ribosomal protein L25 /FUNCTION = Adaptation, protection; Translation, post-translational modification, degradation (SEQ ID No: 662)
PA4272/GENE = rplJ /DEF = 50S ribosomal protein L10 /FUNCTION = Translation, post-translational modification, degradation (SEQ ID No: 663)
PA2619/GENE = infA /DEF = initiation factor /FUNCTION = Translation, post-translational modification, degradation (SEQ ID No: 664)
PA4241/GENE = rpsM /DEF = 30S ribosomal protein S13 /FUNCTION = Translation, post-translational modification, degradation (SEQ ID No: 665)
PA4482/GENE = gatC /DEF = Glu-tRNA (Gln) amidotransferase subunit C /FUNCTION = Translation, post-translational modification, degradation (SEQ ID No: 666)
PA2743/GENE = infC /DEF = translation initiation factor IF-3 /FUNCTION = Translation, post-translational modification, degradation (SEQ ID No: 667)
PA3742/GENE = rplS /DEF = 50S ribosomal protein L19 /FUNCTION = Translation, post-translational modification, degradation (SEQ ID No: 668)
PA5491/DEF = probable cytochrome /FUNCTION = Energy metabolism (SEQ ID No: 669)
PA4238/GENE = rpoA /DEF = DNA-directed RNA polymerase alpha chain /FUNCTION = Transcription, RNA processing and degradation (SEQ ID No: 670)
PA2321/DEF = gluconokinase /FUNCTION = Carbon compound catabolism; Energy metabolism (SEQ ID No: 671)
PA5276 /GENE = lppL /DEF = lipopeptide LppL precursor /FUNCTION = Cell wall/LPS/capsule (SEQ ID No: 672)
PA3743/GENE = trmD /DEF = tRNA (guanine-N1)-methyltransferase /FUNCTION = Transcription, RNA processing and degradation (SEQ ID No: 673)
PA2639/GENE = nuoD /DEF = NADH dehydrogenase I chain C, D /FUNCTION = Energy metabolism (SEQ ID No: 674)
PA1800/GENE = tig /DEF = trigger factor /FUNCTION = Cell division; Chaperones & heat shock proteins (SEQ ID No: 675)
PA5555/GENE = atpG /DEF = ATP synthase gamma chain /FUNCTION = Energy metabolism (SEQ ID No: 676)
PA2970/GENE = rpmF /DEF = 50S ribosomal protein L32 /FUNCTION = Translation, post-translational modification, degradation (SEQ ID No: 677)
PA1582/GENE = sdhD /DEF = succinate dehydrogenase (D subunit) /FUNCTION = Energy metabolism (SEQ ID No: 678)
PA4246/GENE = rpsE /DEF = 30S ribosomal protein S5 /FUNCTION = Translation, post-translational modification, degradation (SEQ ID No: 679)
PA0579/GENE = rpsU /DEF = 30S ribosomal protein S21 /FUNCTION = Hypothetical, unclassified, unknown (SEQ ID No: 680)
PA2634/DEF = probable isocitrate lyase /FUNCTION = Putative enzymes (SEQ ID No: 681)
PA1581/GENE = sdhC /DEF = succinate dehydrogenase (C subunit) /FUNCTION = Energy metabolism (SEQ ID No: 682)
PA5557/GENE = atpH /DEF = ATP synthase delta chain /FUNCTION = Energy metabolism (SEQ ID No: 683)
tRNA_Glycine, 4785688-4785761 (−) strand (SEQ ID No: 684)
PA5298/DEF = xanthine phosphoribosyltransferase /FUNCTION = Nucleotide biosynthesis and metabolism (SEQ ID No: 685)
PA4846/GENE = aroQ1 /DEF = 3-dehydroquinate dehydratase /FUNCTION = Amino acid biosynthesis and metabolism (SEQ ID No: 686)
PA0493/DEF = probable biotin-requiring enzyme /FUNCTION = Putative enzymes (SEQ ID No: 687)
PA3266/GENE = capB /DEF = cold acclimation protein B /FUNCTION = Adaptation, protection; Transcriptional regulators (SEQ ID No: 688)
PA4267/GENE = rpsG /DEF = 30S ribosomal protein S7 /FUNCTION = Translation, post-translational modification, degradation (SEQ ID No: 689)
PA4748/GENE = tpiA /DEF = triosephosphate isomerase /FUNCTION = Central intermediary metabolism; Energy metabolism (SEQ ID No: 690)
PA4847/GENE = accB /DEF = biotin carboxyl carrier protein (BCCP) /FUNCTION = Fatty acid and phospholipid metabolism (SEQ ID No: 691)
tRNA_Valine, 3650815-3650890 (−) strand (SEQ ID No: 692)
PA4249/GENE = rpsH /DEF = 30S ribosomal protein S8 /FUNCTION = Translation, post-translational modification, degradation (SEQ ID No: 693)
PA0856/DEF = hypothetical protein /FUNCTION = Hypothetical, unclassified, unknown (SEQ ID No: 694)
PA5569/GENE = rnpA /DEF = ribonuclease P protein component /FUNCTION = Translation, post-translational modification, degradation (SEQ ID No: 695)
PA0896/GENE = aruF /DEF = arginine/ornithine succinyltransferase Al subunit /FUNCTION = Amino acid biosynthesis and metabolism (SEQ ID No: 696)
PA4430/DEF = probable cytochrome b /FUNCTION = Energy metabolism (SEQ ID No: 697)
PA4935/GENE = rpsF /DEF = 30S ribosomal protein S6 /FUNCTION = Translation, post-translational modification, degradation (SEQ ID No: 698)
PA4031/GENE = ppa /DEF = inorganic pyrophosphatase /FUNCTION = Central intermediary metabolism (SEQ ID No: 699)
PA2744/GENE = thrS /DEF = threonyl-tRNA synthetase /FUNCTION = Amino acid biosynthesis and metabolism; Translation, post-translational modification, degradation (SEQ ID No: 700)
PA4942/GENE = hflK /DEF = protease subunit HflK /FUNCTION = Cell division; Translation, post-translational modification, degradation (SEQ ID No: 701)
PA1557/DEF = probable cytochrome oxidase subunit (cbb3-type) /FUNCTION = Energy metabolism (SEQ ID No: 702)
PA4406/GENE = lpxC /DEF = UDP-3-O-acyl-N-acetylglucosamine deacetylase /FUNCTION = Cell wall / LPS / capsule (SEQ ID No: 703)
PA3621/GENE = fdxA /DEF = ferredoxin I /FUNCTION = Energy metabolism (SEQ ID No: 704)
PA3644/GENE = lpxA /DEF = UDP-N-acetylglucosamine acyltransferase /FUNCTION = Cell wall / LPS / capsule (SEQ ID No: 705)
PA4053/GENE = ribE /DEF = 6,7-dimethyl-8-ribityllumazine synthase /FUNCTION = Biosynthesis of cofactors, prosthetic groups and carriers (SEQ ID No: 706)
PA4276/GENE = secE /DEF = secretion protein SecE /FUNCTION = Protein secretion/export apparatus (SEQ ID No: 707)
PA3811/GENE = hscB /DEF = heat shock protein HscB /FUNCTION = Chaperones & heat shock proteins (SEQ ID No: 708)
PA3645/GENE = fabZ /DEF = (3R)-hydroxymyristoyl-[acyl carrier protein] dehydratase /FUNCTION = Cell wall / LPS / capsule; Fatty acid and phospholipid metabolism (SEQ ID No: 709)
PA4431/DEF = probable iron-sulfur protein /FUNCTION = Putative enzymes (SEQ ID No: 710)
PA1156/GENE = nrdA /DEF = ribonucleoside reductase, large chain /FUNCTION = Nucleotide biosynthesis and metabolism (SEQ ID No: 711)
PA4762/GENE = grpE /DEF = heat shock protein GrpE /FUNCTION = DNA replication, recombination, modification and repair; Chaperones & heat shock proteins (SEQ ID No: 712)
PA3159/GENE = wbpA /DEF = probable UDP-glucose/GDP-mannose dehydrogenase WbpA /FUNCTION = Cell wall / LPS / capsule; Putative enzymes (SEQ ID No: 713)
PA4944/DEF = conserved hypothetical protein /FUNCTION = Hypothetical, unclassified, unknown (SEQ ID No: 714)
PA3636/GENE = kdsA /DEF = 2-dehydro-3-deoxyphosphooctonate aldolase /FUNCTION = Energy metabolism; Translation, post-translational modification, degradation; Carbon compound catabolism (SEQ ID No: 715)
PA4261/GENE = rplW /DEF = 50S ribosomal protein L23 /FUNCTION = Translation, post-translational modification, degradation (SEQ ID No: 716)
PA4252/GENE = rplX /DEF = 50S ribosomal protein L24 /FUNCTION = Translation, post-translational modification, degradation (SEQ ID No: 717)
PA4745/GENE = nusA /DEF = N utilization substance protein A /FUNCTION = Transcription, RNA processing and degradation (SEQ ID No: 718)
PA3635/GENE = eno /DEF = enolase /FUNCTION = Energy metabolism; Translation, post-translational modification, degradation; Carbon compound catabolism (SEQ ID No: 719)
PA0336/DEF = conserved hypothetical protein /FUNCTION = Hypothetical, unclassified, unknown (SEQ ID No: 720)
Intergenic region between PA4674 and PA4675, 5207621-5208463, (+) strand (SEQ ID No: 721)
PA5558/GENE = atpF /DEF = ATP synthase B chain /FUNCTION = Energy metabolism (SEQ ID No: 722)
PA2968/GENE = fabD /DEF = malonyl-CoA-[acyl-carrier-protein] transacylase /FUNCTION = Fatty acid and phospholipid metabolism (SEQ ID No: 723)
PA3832/GENE = holC /DEF = DNA polymerase III, chi subunit /FUNCTION = DNA replication, recombination, modification and repair (SEQ ID No: 724)
PA4483/GENE = gatA /DEF = Glu-tRNA (Gln) amidotransferase subunit A /FUNCTION = Translation, post-translational modification, degradation (SEQ ID No: 725)
PA2747/DEF = hypothetical protein /FUNCTION = Hypothetical, unclassified, unknown (SEQ ID No: 726)
tRNA_Histidine, 1947729-1947804 (+) strand (SEQ ID No: 727)
PA2624/GENE = idh /DEF = isocitrate dehydrogenase /FUNCTION = Energy metabolism (SEQ ID No: 728)
PA2453/DEF = hypothetical protein /FUNCTION = Hypothetical, unclassified, unknown (SEQ ID No: 729)
PA4258/GENE = rplV /DEF = 50S ribosomal protein L22 /FUNCTION = Translation, post-translational modification, degradation (SEQ ID No: 730)
PA4743/GENE = rbfA /DEF = ribosome-binding factor A /FUNCTION = Adaptation, protection; Translation, post-translational modification, degradation (SEQ ID No: 731)
PA1533/DEF = conserved hypothetical protein /FUNCTION = Hypothetical, unclassified, unknown (SEQ ID No: 732)
PA1123/DEF = hypothetical protein /FUNCTION = Hypothetical, unclassified, unknown (SEQ ID No: 733)
PA5490/GENE = cc4 /DEF = cytochrome c4 precursor /FUNCTION = Energy metabolism (SEQ ID No: 734)
PA3001/DEF = probable glyceraldehyde-3-phosphate dehydrogenase /FUNCTION = Putative enzymes (SEQ ID No: 735)
PA1013/GENE = purC /DEF = phosphoribosylaminoimidazole-succinocarboxamide synthase /FUNCTION = Nucleotide biosynthesis and metabolism (SEQ ID No: 736)
PA3987/GENE = leuS /DEF = leucyl-tRNA synthetase /FUNCTION = Amino acid biosynthesis and metabolism; Translation, post-translational modification, degradation (SEQ ID No: 737)
PA4761/GENE = dnaK /DEF = DnaK protein /FUNCTION = Adaptation, protection; Chaperones & heat shock proteins; DNA replication, recombination, modification and repair (SEQ ID No: 738)
PA3701/GENE = prfB /DEF = peptide chain release factor 2 /FUNCTION = Translation, post-translational modification, degradation (SEQ ID No: 739)
PA5128/GENE = secB /DEF = secretion protein SecB /FUNCTION = Protein secretion/export apparatus (SEQ ID No: 740)
PA4253/GENE = rplN /DEF = 50S ribosomal protein L14 /FUNCTION = Translation, post-translational modification, degradation (SEQ ID No: 741)
PA4386 /GENE = groES /DEF = GroES protein /FUNCTION = Chaperones & heat shock proteins (SEQ ID No: 742)
PA5067/GENE = hisE /DEF = phosphoribosyl-ATP pyrophosphohydrolase /FUNCTION = Amino acid biosynthesis and metabolism (SEQ ID No: 743)
PA4880/DEF = probable bacterioferritin /FUNCTION = Central intermediary metabolism (SEQ ID No: 744)
PA1610/GENE = fabA /DEF = beta-hydroxydecanoyl-ACP dehydrase /FUNCTION = Fatty acid and phospholipid metabolism (SEQ ID No: 745)
PA3807/GENE = ndk /DEF = nucleoside diphosphate kinase /FUNCTION = Nucleotide biosynthesis and metabolism (SEQ ID No: 746)
PA4266/GENE = fusA1 /DEF = elongation factor G /FUNCTION = Translation, post-translational modification, degradation (SEQ ID No: 747)
PA0972/GENE = tolB /DEF = TolB protein /FUNCTION = Transport of small molecules (SEQ ID No: 748)
PA4232/GENE = ssb /DEF = single-stranded DNA-binding protein /FUNCTION = DNA replication, recombination, modification and repair (SEQ ID No: 749)
PA3700/GENE = lysS /DEF = lysyl-tRNA synthetase /FUNCTION = Amino acid biosynthesis and metabolism; Translation, post-translational modification, degradation (SEQ ID No: 750)
PA4460/DEF = conserved hypothetical protein /FUNCTION = Hypothetical, unclassified, unknown (SEQ ID No: 751)
PA5069/GENE = tatB /DEF = translocation protein TatB /FUNCTION = Protein secretion/export apparatus (SEQ ID No: 752)
PA4853/GENE = fis /DEF = DNA-binding protein Fis /FUNCTION = DNA replication, recombination, modification and repair; Transcriptional regulators (SEQ ID No: 753)
PA0019/GENE = def /DEF = polypeptide deformylase /FUNCTION = Translation, post-translational modification, degradation (SEQ ID No: 754)
PA0595/GENE = ostA /DEF = organic solvent tolerance protein OstA precursor /FUNCTION = Adaptation, protection (SEQ ID No: 755)
PA4848/GENE = accC /DEF = biotin carboxylase /FUNCTION = Fatty acid and phospholipid metabolism (SEQ ID No: 756)
PA1580/GENE = gltA /DEF = citrate synthase /FUNCTION = Energy metabolism (SEQ ID No: 757)
PA4259/GENE = rpsS /DEF = 30S ribosomal protein S19 /FUNCTION = Translation, post-translational modification, degradation (SEQ ID No: 758)
PA2976/GENE = rne /DEF = ribonuclease E /FUNCTION = Transcription, RNA processing and degradation (SEQ ID No: 759)
PA1574/DEF = conserved hypothetical protein /FUNCTION = Hypothetical, unclassified, unknown (SEQ ID No: 760)
PA2023 /GENE = galU /DEF = UTP--glucose-1-phosphate uridylyltransferase /FUNCTION = Central intermediary metabolism (SEQ ID No: 761)
PA4740/GENE = pnp /DEF = polyribonucleotide nucleotidyltransferase /FUNCTION = Transcription, RNA processing and degradation (SEQ ID No: 762)
PA3907/DEF = hypothetical protein /FUNCTION = Hypothetical, unclassified, unknown (SEQ ID No: 763)
PA2960/GENE = pilZ /DEF = type 4 fimbrial biogenesis protein PilZ /FUNCTION = Motility & Attachment (SEQ ID No: 764)
PA4425/DEF = probable phosphoheptose isomerase /FUNCTION = Putative enzymes (SEQ ID No: 765)
PA5068/GENE = tatA /DEF = translocation protein TatA /FUNCTION = Protein secretion/export apparatus (SEQ ID No: 766)
PA3686/GENE = adk /DEF = adenylate kinase /FUNCTION = Nucleotide biosynthesis and metabolism (SEQ ID No: 767)
PA4759/GENE = dapB /DEF = dihydrodipicolinate reductase /FUNCTION = Amino acid biosynthesis and metabolism (SEQ ID No: 768)
PA4292/DEF = probable phosphate transporter /FUNCTION = Membrane proteins; Transport of small molecules (SEQ ID No: 769)
PA1552/DEF = probable cytochrome c /FUNCTION = Energy metabolism (SEQ ID No: 770)
PA5143/GENE = hisB /DEF = imidazoleglycerol-phosphate dehydratase /FUNCTION = Amino acid biosynthesis and metabolism (SEQ ID No: 771)
PA3014/GENE = faoA /DEF = fatty-acid oxidation complex alpha-subunit /FUNCTION = Amino acid biosynthesis and metabolism; Fatty acid and phospholipid metabolism (SEQ ID No: 772)
PA1505/GENE = moaA2 /DEF = molybdopterin biosynthetic protein A2 /FUNCTION = Biosynthesis of cofactors, prosthetic groups and carriers (SEQ ID No: 773)
PA3981/DEF = conserved hypothetical protein /FUNCTION = Hypothetical, unclassified, unknown (SEQ ID No: 774)
PA2965 /GENE = fabF1 /DEF = beta-ketoacyl-acyl carrier protein synthase II /FUNCTION = Fatty acid and phospholipid metabolism (SEQ ID No: 775)
PA0857/GENE = bolA /DEF = morphogene protein BolA /FUNCTION = Cell division (SEQ ID No: 776)
PA5315/GENE = rpmG /DEF = 50S ribosomal protein L33 /FUNCTION = Translation, post-translational modification, degradation (SEQ ID No: 777)
PA3861/GENE = rhlB /DEF = ATP-dependent RNA helicase RhlB /FUNCTION = Transcription, RNA processing and degradation (SEQ ID No: 778)
tRNA_Asparagine, 3524012-3524087 (+) strand (SEQ ID No: 779)
PA3575/DEF = hypothetical protein /FUNCTION = Hypothetical, unclassified, unknown; Membrane proteins (SEQ ID No: 780)
PA1774 /DEF = hypothetical protein /FUNCTION = Hypothetical, unclassified, unknown; Membrane proteins (SEQ ID No: 781)
PA4333/DEF = probable fumarase /FUNCTION = Energy metabolism (SEQ ID No: 782)
PA2667/DEF = conserved hypothetical protein /FUNCTION = Transcriptional regulators (SEQ ID No: 783)
PA2979/GENE = kdsB /DEF = 3-deoxy-manno-octulosonate cytidylyltransferase /FUNCTION = Cell wall / LPS / capsule (SEQ ID No: 784)
PA4006/DEF = hypothetical protein /FUNCTION = Hypothetical, unclassified, unknown (SEQ ID No: 785)
PA1008/GENE = bcp /DEF = bacterioferritin comigratory protein /FUNCTION = Adaptation, protection (SEQ ID No: 786)
PA4271/GENE = rplL /DEF = 50S ribosomal protein L7 / L12 /FUNCTION = Translation, post-translational modification, degradation (SEQ ID No: 787)
PA3480/DEF = probable deoxycytidine triphosphate deaminase /FUNCTION = Nucleotide biosynthesis and metabolism (SEQ ID No: 788)
PA4503/DEF = probable permease of ABC transporter /FUNCTION = Membrane proteins; Transport of small molecules (SEQ ID No: 789)
PA5119/GENE = glnA /DEF = glutamine synthetase /FUNCTION = Amino acid biosynthesis and metabolism (SEQ ID No: 790)
PA5054/GENE = hslU /DEF = heat shock protein HslU /FUNCTION = Chaperones & heat shock proteins (SEQ ID No: 791)
PA2950/DEF = hypothetical protein /FUNCTION = Hypothetical, unclassified, unknown (SEQ ID No: 792)
PA1609/GENE = fabB /DEF = beta-ketoacyl-ACP synthase I /FUNCTION = Fatty acid and phospholipid metabolism (SEQ ID No: 793)
PA3637/GENE = pyrG /DEF = CTP synthase /FUNCTION = Nucleotide biosynthesis and metabolism (SEQ ID No: 794)
PA5429/GENE = aspA /DEF = aspartate ammonia-lyase /FUNCTION = Amino acid biosynthesis and metabolism (SEQ ID No: 795)
PA5322/GENE = algC /DEF = phosphomannomutase AlgC /FUNCTION = Amino acid biosynthesis and metabolism; Cell wall / LPS / capsule; Secreted Factors (toxins, enzymes, alginate) (SEQ ID No: 796)
PA0429/DEF = hypothetical protein /FUNCTION = Hypothetical, unclassified, unknown (SEQ ID No: 797)
PA3369/DEF = hypothetical protein /FUNCTION = Hypothetical, unclassified, unknown; Membrane proteins (SEQ ID No: 798)
PA4559/GENE = lspA /DEF = prolipoprotein signal peptidase /FUNCTION = Protein secretion/export apparatus; Translation, post-translational modification, degradation (SEQ ID No: 799)
PA1659/DEF = hypothetical protein /FUNCTION = Hypothetical, unclassified, unknown (SEQ ID No: 800)
PA0357/GENE = mutM /DEF = formamidopyrimidine-DNA glycosylase /FUNCTION = DNA replication, recombination, modification and repair (SEQ ID No: 801)
PA5063/GENE = ubiE /DEF = ubiquinone biosynthesis methyltransferase UbiE /FUNCTION = Biosynthesis of cofactors, prosthetic groups and carriers; Energy metabolism (SEQ ID No: 802)
PA0555/GENE = fda /DEF = fructose-1,6-bisphosphate aldolase /FUNCTION = Carbon compound catabolism; Central intermediary metabolism (SEQ ID No: 803)
PA3440/DEF = conserved hypothetical protein /FUNCTION = Hypothetical, unclassified, unknown (SEQ ID No: 804)
PA1009/DEF = hypothetical protein /FUNCTION = Hypothetical, unclassified, unknown (SEQ ID No: 805)
PA1010 /GENE = dapA /DEF = dihydrodipicolinate synthase /FUNCTION = Amino acid biosynthesis and metabolism (SEQ ID No: 806)
PA3626/DEF = conserved hypothetical protein /FUNCTION = Hypothetical, unclassified, unknown (SEQ ID No: 807)
PA1462/DEF = probable plasmid partitioning protein /FUNCTION = Cell division (SEQ ID No: 808)
PA4264/GENE = rpsJ /DEF = 30S ribosomal protein S10 /FUNCTION = Translation, post-translational modification, degradation; Transcription, RNA processing and degradation (SEQ ID No: 809)
PA5078/DEF = conserved hypothetical protein /FUNCTION = Hypothetical, unclassified, unknown (SEQ ID No: 810)
PA0766/GENE = mucD /DEF = serine protease MucD precursor /FUNCTION = Cell wall / LPS / capsule; Putative enzymes; Secreted Factors (toxins, enzymes, alginate) (SEQ ID No: 811)
PA1183/GENE = dctA /DEF = C4-dicarboxylate transport protein /FUNCTION = Transport of small molecules (SEQ ID No: 812)
PA5000/DEF = probable glycosyl transferase /FUNCTION = Putative enzymes (SEQ ID No: 813)
PA3770/GENE = guaB /DEF = inosine-5-monophosphate dehydrogenase /FUNCTION = Nucleotide biosynthesis and metabolism (SEQ ID No: 814)
PA5323/GENE = argB /DEF = acetylglutamate kinase /FUNCTION = Amino acid biosynthesis and metabolism (SEQ ID No: 815)
PA5174/DEF = probable beta-ketoacyl synthase /FUNCTION = Fatty acid and phospholipid metabolism (SEQ ID No: 816)
PA3171/GENE = ubiG /DEF = 3-demethylubiquinone-9 3-methyltransferase /FUNCTION = Biosynthesis of cofactors, prosthetic groups and carriers; Energy metabolism (SEQ ID No: 817)
PA1482/GENE = ccmH /DEF = cytochrome C-type biogenesis protein CcmH /FUNCTION = Energy metabolism (SEQ ID No: 818)
PA2646/GENE = nuoK /DEF = NADH dehydrogenase I chain K /FUNCTION = Energy metabolism (SEQ ID No: 819)
PA2612/GENE = serS /DEF = seryl-tRNA synthetase /FUNCTION = Amino acid biosynthesis and metabolism; Translation, post-translational modification, degradation (SEQ ID No: 820)
PA0527/GENE = dnr /DEF = transcriptional regulator Dnr /FUNCTION = Transcriptional regulators (SEQ ID No: 821)
PA1660/DEF = hypothetical protein /FUNCTION = Hypothetical, unclassified, unknown (SEQ ID No: 822)
PA3962/DEF = hypothetical protein /FUNCTION = Hypothetical, unclassified, unknown (SEQ ID No: 823)
PA3031/DEF = hypothetical protein /FUNCTION = Hypothetical, unclassified, unknown (SEQ ID No: 824)
PA2780/DEF = hypothetical protein /FUNCTION = Hypothetical, unclassified, unknown (SEQ ID No: 825)
PA2649/GENE = nuoN /DEF = NADH dehydrogenase I chain N /FUNCTION = Energy metabolism (SEQ ID No: 826)
PA2644/GENE = nuoI /DEF = NADH Dehydrogenase I chain I /FUNCTION = Energy metabolism (SEQ ID No: 827)
PA0730/DEF = probable transferase /FUNCTION = Putative enzymes (SEQ ID No: 828)
PA4403/GENE = secA /DEF = secretion protein SecA /FUNCTION = Protein secretion/export apparatus (SEQ ID No: 829)
PA3476/GENE = rhlL /DEF = autoinducer synthesis protein RhlL /FUNCTION = Adaptation, protection (SEQ ID No: 830)
PA3286 /DEF = hypothetical protein /FUNCTION = Hypothetical, unclassified, unknown (SEQ ID No: 831)
PA2980/DEF = conserved hypothetical protein /FUNCTION = Hypothetical, unclassified, unknown (SEQ ID No: 832)
PA1642/GENE = selD /DEF = selenophosphate synthetase /FUNCTION = Translation, post-translational modification, degradation (SEQ ID No: 833)
PA0537/DEF = conserved hypothetical protein /FUNCTION = Hypothetical, unclassified, unknown (SEQ ID No: 834)
PA3524/GENE = gloA1 /DEF = lactoylglutathione lyase /FUNCTION = Central intermediary metabolism (SEQ ID No: 835)
PA5134/DEF = probable carboxyl-terminal protease /FUNCTION = Translation, post-translational modification, degradation (SEQ ID No: 836)
PA5038 /GENE = aroB /DEF = 3-dehydroquinate synthase /FUNCTION = Amino acid biosynthesis and metabolism (SEQ ID No: 837)
PA5076/DEF = probable binding protein component of ABC transporter /FUNCTION = Transport of small molecules (SEQ ID No: 838)
PA2528/DEF = probable RND efflux membrane fusion protein precursor /FUNCTION = Transport of small molecules (SEQ ID No: 839)
PA1504/DEF = probable transcriptional regulator /FUNCTION = Transcriptional regulators (SEQ ID No: 840)
PA1102/GENE = fliG /DEF = flagellar motor switch protein FliG /FUNCTION = Motility & Attachment; Cell wall / LPS / capsule (SEQ ID No: 841)
PA1421/GENE = speB2 /DEF = agmatinase /FUNCTION = Amino acid biosynthesis and metabolism (SEQ ID No: 842)
PA0582/GENE = folB /DEF = dihydroneopterin aldolase /FUNCTION = Biosynthesis of cofactors, prosthetic groups and carriers (SEQ ID No: 843)
PA4411/GENE = murC /DEF = UDP-N-acetylmuramate--alanine ligase /FUNCTION = Cell wall / LPS / capsule (SEQ ID No: 844)
PA4054/GENE = ribB /DEF = GTP cyclohydrolase II/3,4-dihydroxy-2-butanone 4-phosphate synthase /FUNCTION = Biosynthesis of cofactors, prosthetic groups and carriers (SEQ ID No: 845)
PA1677/DEF = conserved hypothetical protein /FUNCTION = Hypothetical, unclassified, unknown (SEQ ID No: 846)
PA5224/GENE = pepP /DEF = aminopeptidase P /FUNCTION = Translation, post-translational modification, degradation (SEQ ID No: 847)
PA0943/DEF = hypothetical protein /FUNCTION = Hypothetical, unclassified, unknown (SEQ ID No: 848)
PA1420/DEF = hypothetical protein /FUNCTION = Hypothetical, unclassified, unknown (SEQ ID No: 849)
PA5064/DEF = hypothetical protein /FUNCTION = Hypothetical, unclassified, unknown (SEQ ID No: 850)
PA4423/DEF = conserved hypothetical protein /FUNCTION = Hypothetical, unclassified, unknown (SEQ ID No: 851)
PA3566/DEF = conserved hypothetical protein /FUNCTION = Hypothetical, unclassified, unknown (SEQ ID No: 852)
PA0900/DEF = hypothetical protein /FUNCTION = Hypothetical, unclassified, unknown (SEQ ID No: 853)
PA2953/DEF = electron transfer flavoprotein-ubiquinone oxidoreductase /FUNCTION = Energy metabolism (SEQ ID No: 854)
PA5344/DEF = probable transcriptional regulator /FUNCTION = Transcriptional regulators (SEQ ID No: 855)
PA4345/DEF = hypothetical protein /FUNCTION = Hypothetical, unclassified, unknown (SEQ ID No: 856)
PA3262/DEF = probable peptidyl-prolyl cis-trans isomerase, FkbP-type /FUNCTION = Translation, post-translational modification, degradation; Chaperones & heat shock proteins (SEQ ID No: 857)
PA5227/DEF = conserved hypothetical protein /FUNCTION = Hypothetical, unclassified, unknown (SEQ ID No: 858)
PA0083/DEF = conserved hypothetical protein /FUNCTION = Hypothetical, unclassified, unknown (SEQ ID No: 859)
PA2379/DEF = probable oxidoreductase /FUNCTION = Putative enzymes (SEQ ID No: 860)
PA5479/GENE = gltP /DEF = proton-glutamate symporter /FUNCTION = Membrane proteins; Transport of small molecules (SEQ ID No: 861)
PA2322/DEF = gluconate permease /FUNCTION = Transport of small molecules (SEQ ID No: 862)

TABLE 8

| | % P flight- | | | | sig-Mann- | | | |
|---|---|---|---|---|---|---|---|---|
| | Percent | | sig-T-Test | sig-Mann- | Whitney | | | |
| | sig-T- | | Change | Whitney | Change | % I/MI- | sig log ratio- | sig log ratio- |
| | Test | P Value | Direction | P Value | Direction | Percent | Average | Median |
| PA0523_norC_at (SEQ ID No: 863) | 100 | 0.191 | None | 0.05 | Up | 88 | 0.28 | 0.92 |
| PA0524_norB_at (SEQ ID No: 864) | 100 | 0.094 | None | 0.05 | Up | 88 | −0.36 | 0.25 |

What is claimed is:

1. A method of modifying a phenotypic characteristic of a microorganism, comprising culturing said microorganism in a low sedimental shear environment and harvesting said microorganism from the culture.

2. The method of claim 1, wherein said low sedimental shear environment is spaceflight.

3. The method of claim 2, wherein said low sedimental shear environment is provided by a rotating wall vessel bioreactor.

4. The method of claim 1, wherein said microorganism is selected from bacteria, fungi, viruses, protozoa, protists and worms.

5. The method of claim 4, wherein said microorganism is selected from the group consisting of Salmonella sp., Streptococcus pneumoniae, Pseudomonas aeruginosa, Candida albicans, and Saccharomyces cerevisiae.

6. The method of claim 1, wherein said phenotypic characteristic of said microorganism is selected from the group consisting of virulence, immunogenicity, stress resistance, resistance to a drug or disinfectant, and biofilm formation in culture.

7. The method of claim 1, wherein the virulence of said microorganism is increased as a result of the culturing.

8. The method of claim 1, wherein the immunogenicity of said microorganism is increased as a result of the culturing.

9. The method of claim 1, wherein the stress resistance of said microorganism is altered as a result of the culturing.

10. The method of claim 1, wherein biofilm formation in culture by said microorganism is increased as a result of the culturing.

11. The method of claim 1, wherein the fluid shear level in said environment is adjusted to be 100 dynes per cm² or lower.

12. A method of modifying a phenotypic characteristic of a microorganism in a low sedimental shear environment, comprising altering the concentrations of one or more ions to which said microorganism is exposed to in said environment.

13. The method of claim 12, wherein said low sedimental shear environment is spaceflight.

14. The method of claim 12, wherein said low sedimental shear environment is provided by a rotating wall vessel bioreactor.

15. The method of claim 12, wherein said low sedimental shear environment is an environment within a host during infection by said microorganism.

16. The method of claim 12, wherein said ions are selected from the group consisting of phosphate, chloride, sulfate/sulfur, bromide, nitrate-n, o-phosphate, pH/hydrogen ion, calcium, chromium, copper, iron, lithium, fluoride, magnesium, manganese, molybdenum, nickel, potassium, sodium and zincions.

17. The method of claim 12, wherein said microorganism is selected from bacteria, fungi, viruses, protozoa, protists and worms.

18. The method of claim 17, wherein said microorganism is selected from the group consisting of Salmonella sp., Streptococcus pneumoniae, Pseudomonas aeruginosa, Candida albicans, and Saccharomyces cerevisiae.

19. The method of claim 12, wherein said phenotypic characteristic of said microorganism is selected from the group consisting of virulence, immunogenicity, stress resistance, resistance to a drug, and biofilm formation in culture.

20. A microorganism harvested from a culture of said microorganism grown in a low sedimental shear environment.

21. The microorganism of claim 20, wherein said low sedimental shear environment is spaceflight or provided by a rotating wall vessel bioreactor.

22. The microorganism of claim 20, wherein said microorganism is selected from bacteria, fungi, viruses, protozoa, protists and worms.

23. The microorganism of claim 22, wherein said microorganism is selected from the group consisting of Salmonella sp., Streptococcus pneumoniae, Pseudomonas aeruginosa, Candida albicans, and Saccharomyces cerevisiae.

24. The microorganism of claim 20, wherein said microorganism is an attenuated vaccine strain.

25. A therapeutic composition comprising the microorganism according to any one of claims 20-24.

26. A method of identifying a gene of a microorganism which modulates the response of said microorganism to a low sedimental shear environment, comprising culturing said microorganism a low sedimental shear environment, comparing expression of candidate genes in said microorganism in said low sedimental shear environment relative to control sedimental shear environment, identifying said gene based on differential expression of said gene.

27. The method of claim 26, wherein said low sedimental shear environment is a spaceflight or provided by a rotating wall vessel bioreactor.

28. The method of claim 1, wherein said gene is selected from the group consisting of virulence genes, iron metabolism genes, ion response or utilization genes, cell surface polysaccharide genes, protein secretion genes, flagellar genes, stress genes, genes coding for ribosomal proteins, genes coding for fimbrial proteins, transcriptional regulator genes, genes involved in extracellular matrix/biofilm synthesis, stress response genes, sigma factors, genes encoding RNA binding proteins, genes encoding small noncoding regulatory RNAs (small RNAs), DNA polymerase genes, RNA polymerase genes, plasmid transfer/conjugation genes, genes encoding chaperone proteins, carbon utilization genes, metabolic pathway genes, energy metabolism genes, chemotaxis genes, genes encoding heat shock proteins, genes encoding putative proteins, genes encoding recombination proteins, genes encoding transport system proteins, genes encoding membrane proteins, genes encoding cell wall components (including LPS), housekeeping genes, genes encoding structural proteins and enzymes, and plasmid genes.

29. The method of claim 28, wherein said gene encodes a small regulatory RNA binding protein or a regulatory RNA.

30. The method of claim 26, wherein gene expression is determined in a microarray analysis of mRNA, RT-PCR, qRT-PCR, Western blot analysis, and proteomic analysis.

31. The method of claim 26, further determining whether said gene is involved in establishing infection of said microorganism by generating a mutant microorganism which comprises an inactivating mutation in said gene, and assessing the infectivity of said mutant microorganism in a host.

32. The method of claim 31, wherein said host is selected from the group consisting of an animal or an animal analog, a plant, and a cell or tissue culture.

33. A vaccine composition comprising a microorganism which has been modified by inactivating a gene involved in establishing infection, wherein said gene has been identified according to the method of claim 31.

34. The vaccine composition of claim 33, wherein said microorganism is Salmonella sp., and said gene is Hfq.

35. A method of assessing the efficacy of a candidate compound against infection by a microorganism, comprising culturing said microorganism in a low sedimental shear environment, contacting said microorganism in the culture with said compound, and determining the inhibitory effect of said compound on the growth of said microorganism as indicative of the therapeutic efficacy of said compound.

36. A method of assessing interactions between a host and a microorganism pathogen or an attenuated vaccine strain, comprising placing said host in contact with said microorganism pathogen or said attenuated vaccine strain in a low sedimental shear environment, and evaluating interactions between said host and said microorganism pathogen or said attenuated vaccine strain in said environment.

37. The method of claim 36, wherein said microorganism pathogen has been cultured in said environment prior to said contact.

38. The method of claim 36, wherein said attenuated vaccine strain is a recombinant attenuated vaccine strain.

39. The method of claim 36, wherein said host is selected from the group consisting of animals, animal analogs, plants, and cell and/or tissue cultures from animals, animal analogs or plants.
